(19) United States
(12) Patent Application Publication
(10) Pub. No.: US 2006/0223812 A1
(43) Pub. Date: Oct. 5, 2006

(54) Treating neurodegenerative conditions

(75) Inventors: Eckhard Mandelkow, Hamburg (DE); Eva-Maria Mandelkow, Hamburg (DE); Jacek Biernat, Schenfeld (DE); Martin von Bergen, Gulzow (DE); Marcus Pickhardt, Hamburg (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: MAX-PLANCK-GESELLSCHAFT ZUR FORDERUNGDER WISSENSCHAFTEN, E.V., Munchen (DE)

(21) Appl. No.: 11/351,884

(22) Filed: Feb. 10, 2006

Related U.S. Application Data
(63) Continuation-in-part of application No. PCT/EP04/08031, filed on Jul. 17, 2004.
(60) Provisional application No. 60/652,284, filed on Feb. 11, 2005.

(51) Int. Cl. A61K 31/497; A61K 31/506

(52) U.S. Cl. 514/252.11; 514/252.18

(57) ABSTRACT

The present invention relates to the use of compounds capable of inhibiting protein aggregate formation and capable of depolymerising protein aggregates for the preparation of a pharmiaceutical composition for treating neurodegenerative conditions such as Alzheimer disease.

The present invention relates to the use of compounds capable of inhibiting protein aggregate formation and capable of depolymerising protein aggregates for the preparation of a pharmaceutical composition for treating neurodegenerative conditions such as Alzheimer disease.

Alzheimer's disease (AD) is the most common cause of dementia in the middle-aged and the elderly and is responsible for about 50% of all cases of senile dementia in North America and Western Europe (Iqbal, K. and Grundke-Iqbal, I. 1997). In Alzheimer's disease two main proteins or fragments thereof form abnormal polymers (review, Selkoe 2003). Proteins precipitated in amyloid plaques between cells largely consist of polymerised Aβ-peptide. The microtubule-associated protein tau occurs inside the cells and produces neurofibrillary tangles (Lee et al., 2001; Buee et al. 2002). It is believed that these insoluble aggregates or their oligomeric precursors are responsible for the neuronal degeneration that leads to the cognitive impairment typical for the disease. The distribution of the neurofibrillary changes has been used for the staging of Alzheimer's disease (Braak and Braak, 1991) which is part of the guidelines for post mortem diagnosis (Ball and Murdoch, 1997). The Braak staging is based on the appearance of tau in an aggregated state which in addition is chemically modified in several ways (phosphorylation, truncation, glycation; Johnson and Bailey, 2002). Whether these modifications are the cause, consequence, or merely byproducts of neuronal degeneration is still a matter of debate. For example, different kinases and pathways of phosphorylation have been suggested to be responsible for early stages of degeneration in neurons (Brion et al., 2001; Liu et al., 2002; Maccioni et al., 2001; Zhang and Johnson, 2000), but in vitro the phosphorylation of tau does not appear to promote aggregation (Eidenmuller et al., 2001; Schneider et al., 1999). Examples from other protein aggregation diseases suggest that an increase in concentration drives the protein into aggregation which in turn causes the toxic effects (Bonifacio et al., 1996; Goldberg and Lansbury, 2000; Rochet and Lansbury, 2000; Shtilerman et al., 2002). Conversely, measures that reduce the concentration of oligomers and aggregates alleviate the diseases (Beirao et al., 1999; Lambert et al., 2001; Sanchez et al., 2003; Schenk et al., 1999).

There are different views on whether cytotoxicity due to Aβ aggregation is transmitted from outside the cell or acts during the maturation of pre-fibrillary oligomers within the cells (for review see. Glabe, 2001). Since the discovery of inherited tau pathologies (FTDP-17) they were studied in animal and cellular models (for review see Hutton et al., 2001). Considerable progress has been made in creating tau pathology in transgenic mice (Duff et al., 2000; Gotz, 2001; Higuchi et al., 2002a; Higuchi et al., 2002b; Hutton, 2001; Lewis et al., 2000) or other organisms (Hall et al., 2000; Jackson et al., 2002; Kraemer et al., 2003; Wittmann et al., 2001), but these do not yet reflect the full spectrum of the human pathology, and it is not clear what role tau protein and its aggregation plays in cytotoxicity.

Much of the evidence for cytotoxicity of intracellular aggregates comes from other neurodegenerative diseases like Parkinson's and Huntington's disease (for reviews see Goedert et al, 1998; Volles and Lansbury, 2003). In Parkinson's disease the cytotoxicity of α-synuclein has recently been traced back to pre-fibrillary oligomers that bind to membranes (Caughey and Lansbury, 2003). In Huntington's disease it is reported that aggregated protein can be found in the nucleus (Bates, 2003), possibly affecting gene transcription, and in a mouse model it was shown that disaggregation of polymers leads to a prolonged life-time (Sanchez et al., 2003).

In the case of tau there is the paradoxon that the protein is intrinsically highly soluble, yet it can aggregate into insoluble polymers. The soluble form of tau is characterised as a natively unfolded protein with mostly random coil conformation, as judged by CD or FTIR-spectroscopy, small angle X-ray scattering, gel filtration and limited proteolysis (Schweers et al., 1994; Friedhoff et al., 1998; von Bergen et al., 2000). However, the tau sequence contains certain motifs that may undergo a conformational change towards β-sheet structure. This can drive the protein into filaments that are indistinguishable from those of Alzheimer's brain. Since the intracellular aggregation of tau in AD correlates with the clinical progression of the disease it seemed likely that inhibition or even reversal of the tau aggregation would protect or rescue the affected neurons.

Substances that inhibit tau formation were consequently identified in the art. U.S. Pat. No. 6,479,528 for example discloses that certain inhibitors of fatty acid oxidation also inhibit tau filament formation. Further, WO 03/007933 discloses naphtoquinone-type compounds and their use to modulate the aggregation of proteins associated with neurodegenerative disease. Wischik et al. (1996) describes inhibition of tau aggregation by phenothiazines.

Surprisingly, the compounds of the present invention were found to be more effectiv or efficient, respectively, in relation to drugs known in the art.

Due to the importance of neurodegenerative conditions today there is a considerable need for new pharmaceutical compositions for the treatment of neurodegenerative conditions. The present invention specifically addresses this problem.

The above problem is solved by the use of compounds capable of inhibiting protein aggregate formation and capable of depolymerising protein aggregates for the preparation of a pharmaceutical composition for treating a neurodegenerative condition.

Using a novel screening assay the inventors surprisingly identified a group of specific compounds that are capable of inhibiting protein aggregate formation and capable of depolymerising pre-formed protein aggregates.

The compounds of the present invention can be piperazines having a molecular weight of about 423.56 to about 509.65.

Compounds of the present invention are capable of inhibiting protein aggregate formation. The feature “capable of inhibiting protein aggregate formation” as used in the present application refers to the inherent activity of a compound to decrease protein aggregate formation in vitro in comparison to a control reaction in the absence of the compounds. The in vitro test is preferably a thioflavine S fluorescence assay, such as the assay illustrated in Example 2 of the present application. A compound is capable of inhibiting protein aggregate formation in that assay if a preferably more than >30%, preferably more than >40%, preferably more than >50%, preferably more than >60%, preferably more than >70%, more preferred >80% and most preferred >90% decrease of the signal is obtained.

Depolymerising pre-formed protein aggregates is a further important aspect of the medical use of the compounds of the present invention. The feature “capable of depolymerising protein aggregates” as used in the present application refers to the inherent activity of a compound to depolymerise protein aggregates in an in vitro assay, such as in the thioflavine S fluorescence assay as illustrated in Example 3. A compound is capable of depolymerising protein aggregates if in comparison to a control reaction in the absence of respective compounds preferably more than >30%, preferably more than >40%, preferably more than >50%, preferably more than >60%, more preferred >70% and most preferred >80% decrease of the signal is obtained.

In a preferred embodiment of the present invention the compounds are used to inhibit protein aggregates that comprise paired helical filaments (PHFs) consisting of tau protein. The tau protein belongs to a class of microtubule-associated proteins (MAPs) expressed in mammalian brain that regulate the extensive dynamics and rearrangement of the microtubule networks in the cells. The abnormal aggregation of tau in the form of PHFs is one of the hallmarks of Alzheimer's disease. Aggregation occurs in the cytoplasm and will therefore be toxic for neurons.

According to a further preferred embodiment the compounds are used to inhibit protein aggregates comprising Aβ protein, prion protein, α-synuclein, serum amyloid, transthyretin, huntingtin, insulin or antibody light chain.

According to an especially preferred aspect the present invention is directed to the above medical use of a compound having the following general formula:

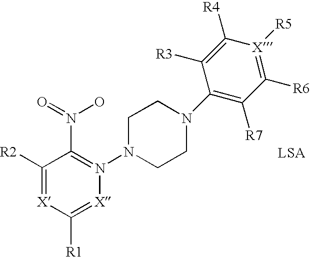

wherein R1 and R2 is selected from H and

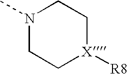

and R3 is selected from H, OCH₃ and F.

R4 is selected from H and CH₃, or R3 and R4 are connected to form a condensed pyrrole ring.

R5, if any, is selected from H and OCH₃.

R6 may be H and R7 may be H, or R6 and R7 may be connected to form a condensed phenyl ring.

R8 is selected from CH₂CH₂OH, CH₂Ph and C(O)OCH₂CH₃, and X′, X″, X′″, and X″″ are selected from N and C.

The compound preferably has one of the following formulas:

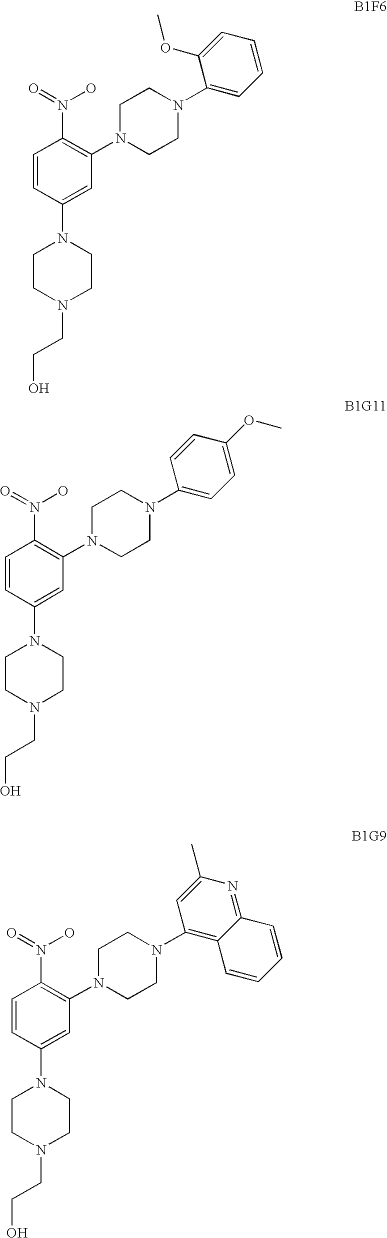

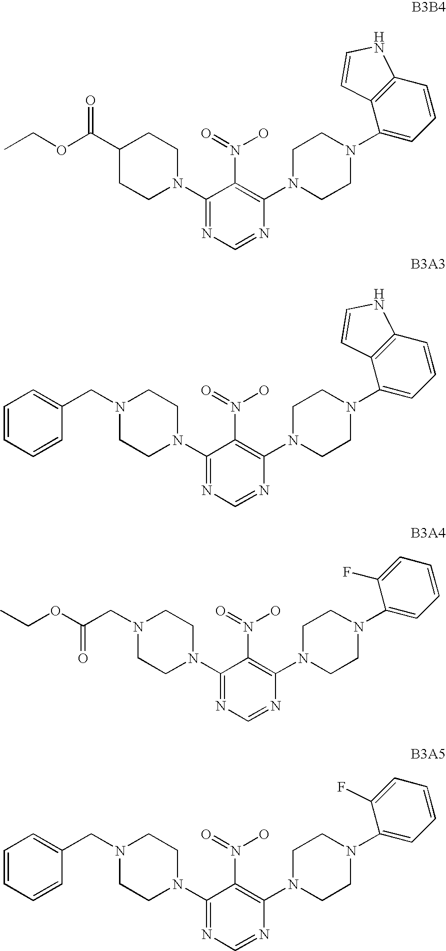

In an alternative embodiment of the invention the above medical use comprises the use of a compound having the following general formula:

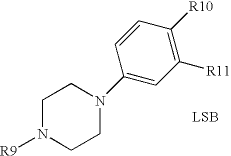

wherein R9 is selected from

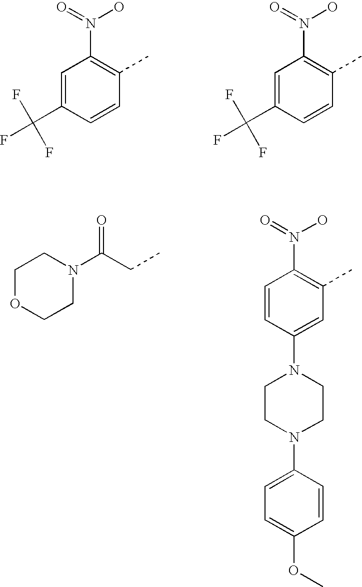

and R10 is selected from H and NO₂.

R11 is selected from an N-morpholino group, N-pyrrolidino group and OCH₃.

In a preferred embodiment the compound may have one of the formulas:

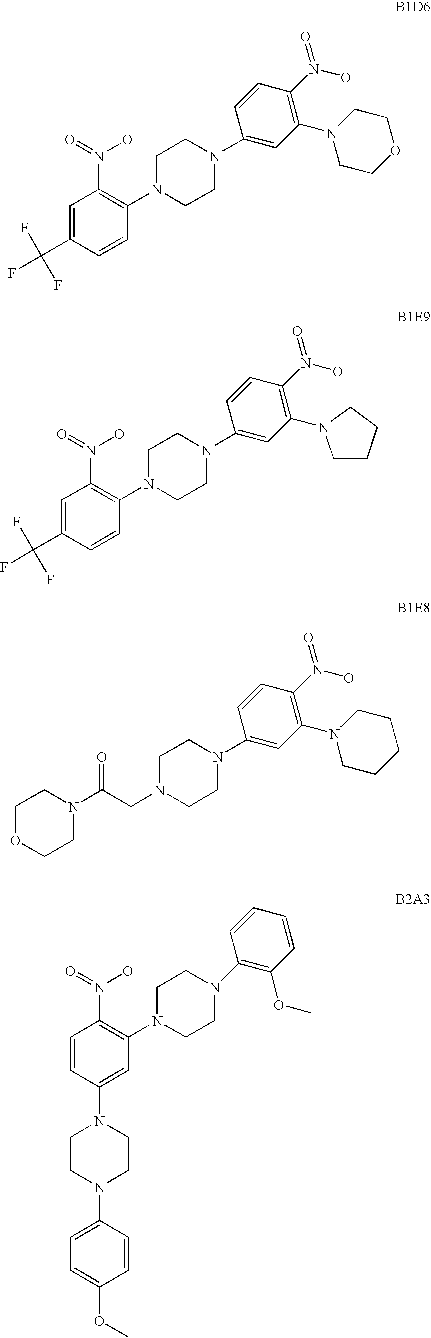

According to a further aspect the invention is directed to the use of compounds capable of inhibiting protein aggregate formation and capable of depolymerising protein aggregates for the preparation of a pharmaceutical composition for treating a neurodegenerative condition, wherein the compound is selected from the group of compounds listed in Table 1 or Table 3.

The present invention is based on a method of screening for compounds that are capable of inhibiting PHF formation and capable of depolymerising PHFs. Briefly such a method can be described as follows.

First a random library is screened for identifying compounds that are capable of inhibiting protein aggregate formation and, capable of depolymerising protein aggregates. Any assay suitable for assessing the capability of inhibiting protein aggregate formation or the capability of depolymerising pre-formed protein aggregates can be used.

The compounds that are identified as compounds capable of inhibiting protein aggregate formation and capable of depolymerising protein aggregates, are then used for carrying out an in silico search to identify potential further compounds. In a second in vitro screen these potential new candidates are tested for their capability to inhibit protein aggregate formation and/or depolymerise protein aggregates.

For example, the described method may comprise the following steps:

In a first screen initially a thioflavine S assay (Example 2) is used to screen a random library for identifying compounds which are capable of inhibiting the PHF formation. The assay is based on the fluorescence of thioflavine S that is increased by binding to PHFs.

The compounds identified are then tested using a thioflavine S assay for their ability to depolymerise pre-formed PHFs (Example 3) in a second step.

Additional assays can be used for testing the ability to inhibit PHF formation or the ability to depolymerise PHFs. Such assays comprise the tryptophan fluorescence assay (Li et al., 2002). This assay is independent of exogenous dyes. It relies on the change of the emission maximum of a tryptophan introduced instead of tyrosine 310 whose emission maximum is sensitive to the burial in a more hydrophobic surrounding upon PHF formation (see Example 6).

Further assays suitable for the present invention are electron microscopy, filter assay or a pelleting assay. Electron microscopy has been used previously to analyse PHF formation (Wille et al. 1992; Schweers et al, 1995; Friedhoff et al, 1998a; Friedhoff et al., 1998b) A filter assay has first been used for the analysis of huntingtin aggregates (Heiser et al., 2000), recently an application for tau-aggregates has been reported (Dou et al., 2003).

In the next step compounds that inhibit PHF formation and depolymerise PHFs are used to define patterns for an in silico homology search of a virtual library of chemical structures. For obtaining reasonable results from an in silico search, compounds which should build the basis of the search have to be selected carefully and several parameters have to be defined.

The compounds were selected with regard to common three dimensional properties (lipophilie, shape and HH-binding ability) and chemical stability. Compounds with a molecular weight higher than 500 were excluded as well as structures with highly polar and reactive groups, for example SH-groups, halide and azo-structures. The number of freely rotateable bonds was minimized.

In a preferred embodiment the compounds are selected with regard to common three-dimensional structure (e.g., shape and binding activity) and chemical stability. Parameters such as size, number of freely rotatable bonds, and inclusion or exclusion of specific groups, such as highly polar or reactive groups, should be defined. In a preferred embodiment of the invention, compounds with a molecular weight higher than 500 are excluded as well as structures with highly polar and reactive groups—for example SH-groups, halide and azo structures, and the number of freely rotatable bonds is minimised.

The compounds, identified by the in silico search, are subsequently tested in vitro for their ability to inhibit PHF formation and depolymerise PHFs with the above methods.

As shown in Example 13, using this strategy leads to a substantial increase of the fraction of compounds that are capable of depolymerising protein aggregates (FIG. 13).

The present invention is further directed to the preparation of pharmaceutical compositions for the treatment of neurodegenerative-conditions.

In a preferred embodiment the neurodegenerative condition is Alzheimer's disease. Alzheimer's disease is characterised by two characteristic types of protein deposits, the first type consists of amyloid precursor protein (APP) and the second type of neurofibrillary tangles of paired helical filaments (PHFs). The compounds and pharmaceutical compositions of the present invention are particularly suitable for the treatment of Alzheimer's disease.

In a further preferred embodiment the present invention is directed to the use of a compound of formula LSA (above) for the preparation of a pharmaceutical composition for treating Alzheimer's disease.

In an alternative embodiment the invention is directed to the use of a compound of formula LSB (above) for the preparation of a pharmaceutical composition for treating Alzheimer's disease.

In yet another embodiment the invention is directed to the use of a compound selected from the group of compounds shown in Table 1 for the preparation of a pharmaceutical composition for treating Alzheimer's disease.

The invention further contemplates the medical use of the compounds for treating other neurodegenerative conditions, such as those selected from the group of tauopathies consisting of CBD (Cortical Basal Disease), PSP (Progressive Supra Nuclear Palsy), Parkinsonism, FTDP-17 (Fronto-Temporal Dementia with Parkinsonism linked to chromosome 17), Familiar British Dementia, Prion Disease (Creutzfeld Jakob Disease) and Pick's Disease.

The term “taupathies” as used herein refers to pathologies characterized by aggregated tau into paired helical filaments leading to neurodegeneration.

According to the present invention the pharmaceutical composition may be administered orally or parenterally.

In a further aspect the invention is directed to the use of the compounds for the preparation of a pharmaceutical composition that is administered as part of a sustained release formulation resulting in slow release of the compound following administration. Such formulations are well known in the art and may generally be prepared using well known technology, for example, by implantation at the desired target site, e.g. in the brain (Sheleg et al., 2002).

The pharmaceutical compositions of the invention may comprise additional compounds such as a pharmaceutically acceptable carrier, diluents, stabilising agents, solubilisers, preserving agents, emulsifying agents and the like.

The invention also comprises a transgenic non-human animal which expresses mutants of tau that polymerize in neurons into aggregates, which aggregates can be visualized by thioflavine S. The transgenic non-human animal-is preferably a transgenic mouse, a rat, a guinea pig and the like.

The transgenic mice allow the expression of human tau isoforms (or mutants thereof) or its domains in the central nervous system (CNS) of mice to determine the effects of tau overexpression. Examples are the effects on the intracellular transport of vesicles and cell organelles in neurons, on the binding of tau to microtubules, and on the aggregation of tau into Alzheimer paired helical filaments (PHFs). The transgenic mice can be obtained for example by following the method of Example 14.

The present invention further relates to cell lines which were genetically modified such that Tau gene expression can be induced. Respective Tau transgenic cell lines have a genetic switch that can be operated at will and that permits the control of the Tau gene activity, quantitatively and reversibly in a temporal, spatial, and tissue-specific manner. These cell lines may further be modified to express mutants of tau that polymerize in neurons into aggregates, which aggregates can be visualized by thioflavine S.

Conditional expression of genes in eukaryotic cell systems and mice can be achieved by the tet-regulated system (Furth et al., 1994). The regulation is done through the tetracycline-regulated transactivator (tTA) (Gossen et al., 1995). FIG. 14 (adapted from Gossen et al., 1995) illustrates the mechanism of action of the Tc-controlled transactivator by the tetracyclin derivative doxycyclin (Dox).

The rtTA system is a variant of the tTA system. It is identical with the exception of 4 amino acid exchanges in the tetR moiety. These changes convey a reverse phenotype to the repressor (rtetR). The resulting rtTA requires doxycyclin for binding to tetO and thus for transcription activation (Gossen et al., 1995). Tissue specificity of these systems is achieved by placing the tTA or rtTA gene under the control of a tissue specific promoter (Pₛₚ), for example the CaMKIIα-promotor for expression in the CNS.

The invention also comprises inducible cell lines for studying the aggregation of Tau protein that is characteristic of Alzheimer's disease and related tauopathies. This allows one to study the toxicity of Tau in cells either in the soluble or aggregated state, the dissolution of Tau aggregates after switching off the Tau gene expression, and the efficiency of small molecule aggregation inhibitors identified by an in vitro screen.

In a further aspect, the present invention relates to screening methods suitable to identify compounds that may be used as active drugs for the treatment of neurodegenerative conditions. The method may comprise analyzing substances to screen for substances capable of inhibiting protein aggregate formation and/or capable of depolymerising protein aggregates, wherein cells are contacted with the compounds and a decrease in protein aggregate formation or a depolymerisation of protein aggregates is determined and wherein the cells express a mutant of tau in an inducible fashion that polymerizes in the cell into aggregates, that can be visualized by thioflavine S.

In an alternative embodiment the method comprises analyzing substances to screen for substances capable of inhibiting protein aggregate formation and/or capable of depolymerising protein aggregates, wherein the above transgenic non-human animals are contacted with the compounds and a decrease in protein aggregate formation or a depolymerisation of protein aggregates is determined. Again these methods are especially suited to screen for compounds for treating Alzheimers disease and other neurodegenerative diseases such as tauopathies (parkinsonism, fronto temporal dementias, picks disease, corticobasal degeneration, prion disease).

In other words, the invention also covers the use of the above animals for analyzing the neurotoxicity of tau indepently from aggregation or for testing conditions that are designed to attenuate or to inhibit the aggregation process within neurons. The conditions thus tested or screened may be a compound or a protein or an antibody or molecules of other classes, such as fatty acids, nucleotides, ribonucleic acids. This use is preferably implemented for identifying agents suitable for treating Alzheimers disease and other neurodegenerative diseases such as tauopathies (parkinsonism, fronto temporal dementias, picks disease, corticobasal degeneration, prion disease). It may also be implemented for obtaining primary hippocampal cultures for performing this screening or testing uses.

The following Examples illustrate the inhibition of protein aggregate formation and the depolymerisation of pre-formed protein aggregates.

### EXAMPLES

Chemicals and proteins used:

Heparin (average MW of 6000), poly-glutamate (average MW of 600 or 1000), thioflavine S was obtained from Sigma. Full-length tau isoforms htau23, htau24 and constructs of the repeat domain of tau (FIG. 1) were expressed in E. coli and purified by making use of the heat stability and FPLC Mono S (Pharmacia) chromatography as described. The purity of the proteins was analysed by SDS-PAGE, protein concentrations were determined by the Bradford assay. Emodin, Daunorubicin and Adriamycin were obtained from Merck (Germany). PHF016 was obtained from ChemBridge (USA) and PHF005 was obtained from Interchim (France). All experiments presented here were carried out with freshly dissolved compounds.

### Example 1

### PHF Formation in vitro

Assembly of synthetic PHFs of tau protein (K19, 10 μM) was performed at 37° C. in the presence of polyanions (heparin; 5 μM) in 50 mM NH₄Ac, pH 6.8. Assembly was followed either qualitatively by electron microscopy or quantitatively by fluorescence assay using thioflavine S. PHF-formation of tau isoforms htau23 and htau24 was carried out in PBS-buffer pH 7.4, 50 μM protein, and 12.5 μM heparin. The samples were incubated at 50° C. for 10 days. In the case of htau24 and K18, DTT was added at a final concentration of 1 mM each day in order to avoid intra-molecular disulfide crosslinking (Barghorn et al., 2000).

### Example 2

### Screening of Compounds Capable of Inhibiting PHF Formation with the Thioflavine S Assay

PHF formation was monitored by a thioflavine S fluorescence assay (Friedhoff et al., 1998a) adapted to a 384 well format. 60 μM of each substance was tested for its inhibitory effect on PHF formation. Using an automated pipetting system (Cybi-Well, CyBio, Jena, Germany) 50 mM NH₄AC, 10 μM protein (K19), 60 μM compound and 5 μM heparin were mixed in 50 μl volume in a 384 well plate (black microtiter 384 plate round well, ThermoLabsystems, Dreiich, Germany) and incubated overnight at 37° C. As a control the protein was replaced with H₂O to measure the possible fluorescence of the compounds. As a second control the reaction mixture without compound was treated in the same way.

After incubation with the compounds thioflavine S was added to the buffer to a final concentration of 20 μM and the signal was measured at excitation at 440 nm and emission at 521 nm in a spectrofluorimeter (Ascent; Labsystems, Frankfurt).

Hits were defined by a >90% decrease of the signal in comparison to the (second) control reaction without compound.

### Example 3

### Screening of Compounds Capable of Depolymerising PHFs with the Thioflavine S Assay

Depolymerisation of PHFs was monitored by the thioflavine S fluorescence. 60 μM of each compound was tested for its ability to depolymerise pre-formed PHFs. 50 mM NH₄Ac, 10 μM PHF (K19), 60 μM compound and 5 μM heparin were mixed in 50 μl volume in a 384 well plate (black microtiter 384 plate round well, ThermoLabsystems, Dreiich, Germany) and incubated overnight at 37° C. As a control the reaction mixture without compound was treated in the same way.

After incubation thioflavine S was added to the buffer to a final concentration of 20 μM and the signal was measured at excitation at 440 nm and emission at 521 nm in a spectrofluorimeter (Ascent; Labsystems, Frankfurt).

Hits were defined by a >80% decrease of the signal in comparison to the control reaction without compound.

### Example 4

### Inhibition of PHF Formation Using Various Concentrations of Compounds and Various Constructs

This Example describes the ability of the five compounds Adriamycin, Daunorubicin, Emodin, PHF005 and PHF016 (FIGS. 1A-E) to inhibit PHF formation. Additionally to the construct K19 (FIG. 1I) the four repeat construct K18 (FIG. 1H) and the related full length isoforms htau23 (three repeat, no inserts, FIG. 1G) and htau24 (four repeats, no inserts, FIG. 1F) were also used.

Using fixed protein concentrations of K19 the compounds were tested in a concentration range from 0.01 nM to 200 μM (FIG. 2A) and IC₅₀ values were determined (Table 2). Inhibitory effects begun to appear at concentrations around 0.1 μM (ratio of protein to compound=100), and reached nearly complete inhibition at 100 μM compound concentration (ratio protein/compound=0.1). Overall, the curves of FIG. 2A decay fairly steeply over a compound concentration range of 2-3 orders of magnitude. The values of half-maximal inhibition (IC₅₀) ranged from 1.0-17.6 μM, which means that all compounds interfered with PHF aggregation of K19 already at substoichiometric concentrations.

The four repeat construct K18 was tested under the same conditions (FIG. 2B). The compounds exhibited IC₅₀ concentrations between 0.1 and 0.6 μM, except for PHF005 whose IC₅₀ was 2.7 μM. However, the decay of the curves of K18 is more gradual than those of K19, extending over 3-4 orders of magnitude of compound concentration (compare FIG. 2A).

The study was then extended to the natural three and four repeat isoforms htau23 and htau24 (FIGS. 1G, F). PHF formation of these proteins was assayed in the presence of 0.1, 1, 10 and 60 μM compound (FIGS. 2C, D). For htau23 a clear dose dependent inhibition was observed (FIG. 2C). The compounds can be subdivided into two groups. The more effective compounds are adriamycin, daunorubicin and emodin which are capable to inhibit PHF formation about 50% at 0.1 μM and ˜90% at 60 μM. Compounds PHF016 and PHF005 are less inhibitory, they showed only a slight effect at low concentration and a moderate one (˜50%) at 60 μM. In the case of htau24 (4 repeats) the compounds showed generally a lower efficiency of inhibition than for htau23 (FIG. 2D), but the internal ranking stayed the same. The more active compounds emodin, daunorubicin and adriamycin reached inhibition levels of 70-90% at 60 μM concentration. PHF016 and PHF005 showed clear differences in their capacity to influence PHF formation; only a small effect was seen with 4-repeat htau24, compared to htau23.

All the polymerisation reactions described so far used heparin as a cofactor for inducing PHF assembly because otherwise the process would be impracticably slow (Goedert et al., 1996; Perez et al., 1996). In order to rule out a potential influence of heparin on the efficiency of the compounds the 4-repeat construct K18/ΔK280 which carries one of the mutations observed in frontotemporal dementia (van Swieten et al., 1999) and is capable of polymerising into PHFs without a polyanionic cofactor (von Bergen, 2001) (FIG. 2E) was used. The resulting IC₅₀ values for the inhibition of filament formation from K18/ΔK280 were significantly higher than for K18wt. The most effective ones are emodin, adriamycin and PHF016 which ranged from 1.3 to 3.9 μM. Daunorubicin which was very active in the case of K19 exhibited an IC₅₀ of 48 μM and PHF005 which was the least efficient inhibitor of K19 and K18 filament formation failed nearly completely. The differences in inhibition effects for K18 (with heparin) and K18/ΔK280 (without heparin) could be caused either by a difference in conformation and/or protein-protein interactions, or perhaps by an interaction between the compound and the cofactor heparin.

### Example 5

### Depolymerisation of PHFs Using Various Concentrations of Compounds and Various Constructs

The ThS assay was used to analyse the ability of the five compounds (see Example 4) to depolymerise pre-formed PHFs made from the repeat domain constructs K19 and K18 as well as from isoforms htau23 and htau24, containing 3 or 4 repeats, respectively.

The depolymerisation of K19 filaments (FIG. 4A) followed a similar concentration dependence as the inhibition experiment, with consistently similar or slightly higher DC₅₀ values than the corresponding IC₅₀ concentrations (Table 2). The ratios of IC₅₀/DC₅₀ range from 0.2-1.2. By contrast, K18 filaments appeared to be much more stable and therefore depolymerised less readily, resulting in higher DC₅₀ values between ˜6.5 and 43 μM (FIG. 4B). Here, too, the concentration dependence for K19 was steeper than for K18 (compare FIGS. 4A, B), similar to that of assembly inhibition (FIGS. 2A, B). Thus the relationship between assembly inhibition and disassembly promotion (IC₅₀ vs. DC₅₀) was less apparent for K18 than for K19, suggesting that the second repeat R2, present only in K18, confers higher stability to the polymer.

All compounds were also able to dissolve PHFs made from K18/ΔK280 without heparin (FIG. 4E) in a dose dependent manner, but exhibiting higher DC₅₀ values than PHFs made from K18. Similarly, the compounds showed a lower activity in depolymerising PHFs made from K18/ΔK280 (FIG. 4E), compared to inhibition of polymerisation, consistent with the experiments on K19 and K18. Emodin, daunorubicin and adriamycin showed DC₅₀ values between 2.7 and 22.0 μM, whereas the DC50 values of PHF016 and PHF005 are not accurately detectable due the low efficiency of depolymerisation under these conditions. The higher DC₅₀ values for K18/ΔK280 point to the higher stability of PHFs formed by this mutant.

PHFs assembled from the full length three repeat isoform htau23 were also sensitive to disaggregation (FIG. 4C). The DC₅₀ values ranged from 7.0 to 60 μM. All values were higher than the IC₅₀ values, but the internal ranking of the compound stayed the same. Emodin, daunorubicin and adriamycin (DC₅₀ range 7.0-13.2 μM) had a much stronger effect than PHF016 and PHF005 (DC₅₀>60 μM). This is consistent with the similar ranking of compounds in the assembly inhibition assay of full-length tau isoforms (FIGS. 2C, D).

By contrast, even the most potent compounds in depolymerising htau23 filaments (emodin, daunorubicin and adramycin, FIG. 4C) were only weak PHF breakers for htau24 filaments (FIG. 4D). All compounds exhibited a comparable low efficiency, the best values were achieved for PHF016 and PHF005 with DC₅₀ values of 39.2 and 10.8 μM respectively. At the lowest concentration (0.1 μM) none of the compounds was able to decrease the level of ThS fluorescence significantly, whereas at the highest concentration (60 μM) the ThS fluorescence was decreased to a range of 10-55%. PHF016 and PHF005 were more active in depolymerising htau23 than htau24 filaments. This difference can be explained both by an increased stability of four repeat isoforms and by an isoform specific mode of action of the compounds.

TABLE 2
IC₅₀/DC₅₀ values of inhibition of PHF aggregation/depolymerisation of PHFs from tau and tau constructs

| | Inhibition of tau aggregation IC₅₀ in μM | | | | | Depolymerisation of PHFs DC₅₀ in μM | | | | |
|---|---|---|---|---|---|---|---|---|---|---|
| Compound | K19 | K18 | K18/ΔK280 | hTau23 | hTau24 | K19 | K18 | K18/ΔK280 | hTau23 | hTau24 |
| Emodin | 2.4 | 0.3 | 1.9 | 0.2 | 1.8 | 2.0 | 2.0 | 2.7 | 7.0 | >60.0 |
| Daunorubicin | 1.0 | 0.3 | 48.1 | 0.1 | 3.4 | 3.1 | 4.0 | 7.7 | 8.2 | >60.0 |
| Adriamycin | 17.6 | 0.1 | 3.9 | 0.2 | 2.7 | 27.0 | 4.3 | 22.0 | 13.2 | >60.0 |
| PHF016 | 6.8 | 0.6 | 1.3 | 1.0 | >60.0 | 7.8 | 7.3 | >60.0 | >60.0 | 39.2 |
| PHF005 | 6.0 | 2.7 | >60.0 | 1.1 | >100.0 | 9.4 | 20.8 | >100.0 | >60.0 | 10.8 |

### Example 6

### Tryptophan Fluorescence Spectroscopy

In order to exclude a possible distortion of the data by the dye the results of the ThS assays can be confirmed by a tryptophan fluorescence assay (Li et al., 2002). It allows the detection of the molecular environment of a tryptophan introduced instead of tyrosine 310 whose emission maximum is sensitive to the burial in a more hydrophobic surrounding upon PHF formation. Therefore the mutants K19/Y310W (FIG. 1I) and K18/Y310W (FIG. 1H) that contain a single tryptophan within the core of the PHF structure were created. In the soluble protein the emission maximum lies at ˜354 nm, whereas it shifts to 340 nm upon PHF formation (FIG. 3A, compare first and second entry). The emission peak can be shifted back by incubation at high concentrations of GuHCl which is due to the disassembly of the PHFs (FIG. 3A, fourth entry).

The fluorescence experiments were performed on a Spex Fluoromax spectrophotometer (Polytec, Waldbronn, Germany) using 3 mm×3 mm micro cuvettes from Hellma (Mühlheim, Germany) with 20 μl sample volumes. A tryptophan emission spectrum scans from 300 to 450 nm at fixed excitation wavelength of 290 nm. The slit widths were 5 nm, the integration time was 0.25 second, and the photomultiplier voltage was 950 V. For fluorescence inhibition assay, 60 μM compounds were incubated with K19/Y310W construct (10 μM) or K18/ΔK280/Y310W and heparin (2.5 μM) in PBS, pH 7.4 three days at 37° C.

In the Trp fluorescence assay the inhibition of PHF assembly becomes apparent if the emission maximum of Trp310 remains higher than that of the control without any compound, because Trp310 remains in a more solvent-accessible hydrophilic environment. The three repeat tau construct K19 (at 10 μM) was prevented from polymerisation by about 90% by the presence of all compounds at a concentration, of 60 μM (FIG. 3A, note that entries 5-9 retain their values around 354 nm, similar to the control #1). By contrast the four repeat tau construct K18/Y310W was inhibited to this high extent only by PHF005 (FIG. 3B, entry #9). Emodin, daunorubicin and adriamycin could prevent PHF formation to about 70% at 60 μM (FIG. 3B, entries #5, 6, 7), whereas PHF016 achieved only 25% inhibition (#8). The trend becomes even more pronounced in the case of K18/ΔK280, where all compounds showed a lower activity. The internal ranking stays roughly the same as with K18; PHF005 (#9) is the best, PHF016 (#8) the worst inhibitor. Emodin, daunorubicin and adriamycin (#5, 6, 7) showed a level of ˜30-50% inhibition. The apparent degrees of inhibition differ somewhat between the ThS fluorescence and the intrinsic Trp fluorescence assays, but this may be due to the different origins of the signal. In the ThS assay the dye has to bind to the filaments, which requires a minimal length of the fibres. The tryptophan fluorescence assay depends on the local surrounding of the residue and is therefore less dependent on the length of the filaments.

For the fluorescence depolymerisation assay, 60 μM inhibitor compound were added to pre-formed PHFs (10 μM) and incubated overnight at 37° C. PHFs were formed by incubation of tau construct K19/Y310W (50 μM) or K18/ΔK280/Y310W with 12.5 μM heparin in volume of 100 μl at 37° C. in PBS, pH 7.4. Incubation time was three days. The formation of aggregates was observed as a shift of the emission maximum from ˜354 nm to ˜340 nm.

Judging by the tryptophan assay the compounds were able to dissolve K19 filaments (FIG. 5A) with the exception of daunorubicin (FIG. 5A, entry #6). All other compounds yielded emission maxima of the protein after treatment around 350-353 nm, close to the value of soluble tau, indicating a depolymerisation efficiency of about 80-90%. In the case of K18 filaments (FIG. 5B) all compounds showed a significantly lower efficiency of depolymerisation, only PHF005 was a strong inhibitor in these conditions (80%), whereas emodin, adriamycin and PHF016 exhibit not more than 10% efficiency.

This ranking is consistent with the assembly inhibition assay (FIG. 3B); In the case of K18/ΔK280 (FIG. 5C) the efficiency of disassembly was further reduced, but the ranking remains comparable to that of K18 (compare FIG. 5B), as well as to the assembly inhibition assay (FIG. 3C). In these cases, PHF005 remained the most potent agent for depolymerising PHFs (entry #9).

The striking differences to the results obtained by thioflavine S assay can be explained by the different approaches of the assays. It is not known under what conditions ThS binds to PHFs, or how long the filaments have to be to become detectable. In the case of the tryptophan assay the local environment of every tryptophan is measured. It is therefore possible that in the ThS assay the long filaments are overrepresented, or that the tryptophan assay discriminates not between soluble and aggregated forms of tau, but only between more or less hydrophobic environments.

### Example 7

### Filter Trapping Assay

The effect of compounds on the depolymerisation of PHFs was analysed using a filter trapping assay. This assay monitors aggregated tau which is trapped on a membrane filter, whereas the soluble protein is washed through. Therefore the technique preferably detects larger filaments, similar to the ThS assay.

Aggregates of tau were trapped by filtration through a PVDF-membrane (pore diameter 0.45 μm, Schleicher and Schuell, Düren, Germany) adapted to 96-well dot blot apparatus. The PVDF-membrane was wetted with methanol and rinsed with PBS-buffer before incorporated into the dot blot apparatus. The samples were pipetted into 100 μl of PBS and filtered. The membrane was washed three times with PBS before taken out of the apparatus and blocked with 5% milk powder in PBS for 30 minutes in a rotational shaker at room temperature. The polyclonal antibody K9JA was used as primary antibody and incubated at a dilution of 1:20.000 at room temperature for one hour. A secondary anti-rabbit antibody conjugated with horse-radish peroxidase (Dako, Hamburg, Germany) was diluted 1:2000 and incubated for 30 minutes at 37° C. After three times washing with TBS-Tween the signal was detected using the ECL system (Amersham Pharmacia) and pictures were taken with the digital gel documentation system Fuji film BAS3000 (Raytest, Straubenhardt, Germany). Quantification of the signals was performed with the AIDA-software package (Raytest, Straubenhardt, Germany).

Representative results are shown for htau23 (FIG. 5D). The compounds showed similar depolymerising activities as with the ThS assay; emodin was most effective with a DC₅₀ of ˜0.5 μM.

### Example 8

### Depolymerisation of PHFs at Prolonged Incubation Times

Depolymerisation data were typically obtained after 12 hours of incubation, but one is also interested in the effects of longer incubation times and lower compound concentrations which yielded only small effects after 12 hours. FIGS. 7A and 7B show the time course of depolymerisation of K19 PHFs in the presence of 0.5 μM adriamycin or PHF005 during 28 days. Nearly no effects were seen after 12 hours, consistent with the other experiments (FIG. 3A) but interestingly the depolymerisation still continued and resulted in a final depolymerisation of ˜20-30% after 28 days. This result suggests that even low concentrations of inhibitors can be used for depolymerisation using prolonged incubation times.

### Example 9

### Electron Microscopy

Protein solutions diluted to 0.1-10 μM were placed on 600-mesh carbon-coated copper grids for 1 min and negatively stained with 2% uranyl acetate for 45 sec. The specimen was examined in a Philips CM12 electron microscope at 100 kV (Eindhoven, Netherlands).

FIG. 6 shows the electron micrographs of hTau23-PHFs and hTau24-PHFs treated with different compounds for overnight.

### Example 10

### Aggregation of Aβ Fibres

Besides the activity of the compounds towards tau fibres, their specificity is an important issue, i.e. the ability to discriminate between different types of aggregates. Therefore, an analysis of the influence of the compounds (60 μM) on amyloid fibrils made from the Aβ1-40 peptide, both in terms of inhibition of de novo filament formation and depolymerisation was performed (FIGS. 8A-B). These fibres are also abundant in Alzheimer brain and contain a core of cross-β-structure, but are located outside the cells, in contrast to the intracellular PHFs.

Commercial human Aβ1-40 was obtained from Calbiochem (Schwalbach, Germany) and stored at −20° C. The AD peptide was routinely dissolved in 100% DMSO to obtain a 2 mM stock solution, which was subsequently stored frozen at −20° C. 5 μl from the 2 mM Aβ stock solution was added to 90 μl of 25 mM phosphate buffer containing 120 mM NaCl, and 0.02% sodium azide, final pH 7.4 and 5 μl of 100% DMSO so that the final DMSO concentration was 10% v/v, and the protein concentration was adjusted to 100 μM. Incubations were at room temperature. In order to accelerate aggregation tubes were put on a lab shaker and agitated at moderate speed. For analysis 5 μl of this solution were added to 45 μl 10 mM phosphate buffer containing 6 μM thioflavine T, pH 6.0, after 30 minutes incubation at room temperature the fluorescence was measured at 504 nm emission by an excitation of 409 nm. To correlate fibril morphology with the fluorescence signal, aliquots of the Aβ1-40 solutions were simultaneously prepared for electron microscopy. The inhibition of fibril formation and disassembly of pre-formed Aβ-fibrils were carried out in triplicates with 60 μM compound and 10 μM protein.

Most of the compounds showed an inhibition of Aβ filament formation of about 90% (FIG. 8A) and a depolymerisation activity of about 85%, except PHF005 which reached ˜50% inhibition in the assembly and disassembly assay. Thus PHF005 appears to interfere more specifically with filaments made from tau, whereas the other compounds are promiscuous in terms of inhibiting β-sheet structures from different sources.

### Example 11

### Light Scattering for Analysis of the Influence of Tau on Microtubule Assembly

The repeat domain of tau is not only important for PHF aggregation but also for the physiological function of microtubule binding. Microtubule polymerisation assays were performed in the absence and presence of compounds (FIG. 9).

The ability of tau to promote microtubule assembly was monitored by light scattering at 350 nm in a Tecan spectrophotometer model Safire (Tecan, Crailsheim, Germany). Tau protein (10 μM) was mixed with tubulin dimer (30 μM) and GTP (1 mM) at 4° C. in polymerisation buffer (100 mM Na-PIPES pH 6.9, 1 mM EGTA, 1 mM MgSO₄, 1 mM DTT), with a final volume of 40 μl. Htau40 and inhibitor compounds (60 μM) were added last. After rapid mixing, the samples were pipetted into a Greiner transparent flat bottom 384 well plate (4 mm path length), which was prewarmed at 37° C. The reaction was started by incubating the cooled components of the reaction at 37° C. The assembly of tubulin into microtubules was monitored over time by a change in turbidity. Three parameters were extracted from curves. The maximum turbidity at steady state, the rate of assembly, and the lag time between the temperature jump and the start of the turbidity rise.

Tubulin (at 30 μM) without tau serves as a negative control which is unable to self-assemble into microtubules because it is below the critical concentration. However, in the presence of tau (10 μM) tubulin polymerised within 4 min. In the presence of compounds (60 μM) the rate and extent of polymerisation were not significantly affected, except for daunorubicin. The same is true for Congo Red, an Aβ fibre inhibitor (Podlisny et al., 1998), used as a further control. These data suggest that the tested compounds influence specifically the pathological. aggregation of tau protein, but not its interaction with microtubules.

### Example 12

### Assays of Tau Aggregation in Cells

A crucial test for the application of inhibitors is their effect in cell models of tauopathy. A neuroblastoma (N2a) cell line which allows inducible expression of the tau construct K18ΔK280 under the control of the tet-on transactivator was generated. This construct was chosen because it contains the FTDP17 mutation ΔK280 in the 4-repeat domain K18 which promotes the formation of β-structure and therefore aggregates readily, even in the absence of polyanionic inducers (von Bergen et al., 2001; {Barghorn, 2002).

The tau construct K18/ΔK280 was expressed in the mouse neuroblastoma cell line N2a in an inducible manner under the control of the reverse tetracycline-controlled transactivator (rtTA) as described elsewhere (Gossen & Bujard, 2002). The inducible N2a/K18ΔK280 cells were cultured in MEM medium supplemented with 10% fetal calf serum, 2 mM glutamine and 0.1% nonessential amino acids. The expression of K18/ΔK280 was induced by addition 1 μg doxycyclin per 1 ml medium. The effect of aggregation inhibition was observed by adding the inhibitor emodin (15 μM). After 3-7 days the cells were harvested and tested for tau aggregation, thioflavin S fluorescence, and viability.

The levels and solubility of the K18/ΔK280 tau protein were determined by the method of Greenberg and Davies (1990) which makes use of the insolubility of protein aggregates after treatment with sarkosyl. The supernatant and sarcosyl-insoluble pellets were analysed by Western blotting with the pan-tau antibody K9JA and analysed by densitometry. Aggregation of tau in cells was tested by the fluorescence of thioflavine S. ThS signals were scored in three independent fields containing 40 cells each.

FIG. 10A shows SDS blots of the cell extract after 7 days. The pellet of the untreated control (-emodin) shows the typical “smear” at higher molecular weight which is characteristic of aggregation in Alzheimer's disease as well (FIG. 10A, lane 2). However, emodin strongly suppressed the aggregates, leaving tau mostly in the soluble state (FIG. 10A, lane 4). Quantification of the sarkosyl-insoluble fraction showed a 5-fold reduction by emodin, from 14% of the total cellular tau down to 3% (FIG. 10B). Similar results were obtained by staining the cells with ThS (to show aggregated material) and with an antibody against total tau (to show the level of tau expression) (FIG. 11) The levels of tau expression were comparable without or with emodin (compare FIG. 11 left, top and bottom). However, whereas the ThS signal is strong in the tau expressing cells, it becomes very weak in the presence of emodin, consistent with the absence of aggregates (FIG. 11 middle, top and bottom). There were fewer ThS responsive cells, and fluorescence intensity was much lower as well. The merged images illustrate that a large fraction of cells contained visible aggregates (green-yellow in superposition), whereas the ThS signal was hardly visible in the emodin-treated cells (FIG. 11, right). The quantification of the images is shown in FIG. 10C.

### Example 13

### Identification of Compounds Capable of Inhibiting PHF Formation and Capable of Depolymerising Pre-Formed PHFs

In a first screen 200.000 compounds were analysed for their influence on PHF aggregation from the tau construct K19 using the thioflavine S assay as described in Example 2. 1266 compounds—corresponding to 0.6% of the library—were able to inhibit PHF aggregation to an extent higher than 90%. Out of these 1266 compounds, 77 were also able to disassemble PHFs with an efficiency of more than 80% (measured as described in Example 3), corresponding to 0.04% of the total library.

In the next step the 77 best compounds in the experimental PHF depolymerisation assay were used for an in silico search for potential PHF inhibitors. For this in silico search several criteria were set. The compounds were selected with regard to common three-dimensional properties (shape and binding ability) and chemical stability. Compounds with a molecular weight higher than 500 were excluded as well as structures with highly polar and reactive groups—for example SH-groups, halide and azo-structures. The number of freely rotateable bonds was minimised. A search of three million chemical structures yielded 300 compounds, of which 241 were further tested.

The compounds were obtained from different companies, tested for solubility in 100% DMSO and in aqueous buffers and analysed with respect to absorbance and fluorescence. The fluorescence of 66 substances interfered with the ThS assay. Therefore a second screen with 175 compounds was performed by testing their capability to inhibit PHF assembly and for PHF disassembly by the ThS assay.

FIG. 12A shows that the percentage of inhibitory compounds was similar in the first and in the second Thioflavine S screen, whereas the fraction of depolymerising substances was increased >40 fold in the second screen (FIG. 12B). These two observations can be explained by the fact that the in silico search was performed on the basis of the substances that are capable of inhibiting assembly as well as inducing disassembly. The results are confirmed by the analysis of the distribution of efficiencies of inhibition and depolymerisation (FIGS. 13A, B). The results show that the efficiency of inhibition was not altered by the selection of the compounds for the second screen but the average efficiency of depolymerisation was increased.

### Example 14

### Generation of Tau Transgenic Mice

Doubly transgenic mice for the conditional expression of transgenic Tau constructs in the CNS were created by crossing the tTA transgenic mice (where the expression of tTA transactivator is driven by the CAMKII-α promoter, termed CamKIIα-tTA mice) and transgenic mice carrying the tau transgene (termed Tau-BiTetO mice).

Generation of Transgenic Tau-BiTetO Mice:

For the generation of this type of transgenic mice it is necessary to construct plasmids carrying the bidirectional tetO responsive promoter followed by both a tau isoform (or mutant) in one direction and luciferase reporter sequences in the other (Baron at al., 1995). The pBI-5 plasmid-derivatives carrying tau isoforms or mutants were constructed by inserting the tau cDNA sequence containing the ClaI site at 5′ and SalI site at 3′ terminus in the appropriate restriction sites available in the multiple cloning site of the pBI-5 vector.

The pBI-5 plasmid (FIG. 15) was originally constructed in H. Bujard's laboratory (Baron et al., 1995), but is now available from Clontech under the name pBI-L. The bidirectional Tet vectors were used to simultaneously express two genes under the control of a single TRE (tetracycline-responsive element) consisting of seven direct repeats of a 42-bp sequence containing the tetO (tetracycline operator) followed downstream and upstream by the minimal CMV promoter (P_{minCMV})

pBI-L can be used to indirectly monitor the expression of tau protein by following the activity of the reporter gene luciferase expressed at the same time downstream of TRE.

The sequences encoding the Tau isoforms or mutants htau40/ΔK280, htau40/ΔK280/2P, K18/ΔK280 and K18/ΔK280/2P were amplified by PCR from E. coli expresssion vectors pNG-2, (pNG-2/htau40/ΔK280, pNG-2/htau40/ΔK280/2P, pNG-2/K18/ΔK280, and pNG-2/K18/ΔK280/2P) and supplied with ClaI and SalI restriction sites at the N- and C-terminus, respectively. ΔK280 means a deletion of amino acid lysine 280 in the tau protein sequence, with corresponding nucleotides 838-840 deleted from the Tau gene sequence. This Tau mutation was detected in a Dutch family afflicted with frontotemporal dementia, (FTDP-17, Rizzu et al., 1999). As shown previously (Barghorn et al., 2000), this mutant possesses a particularly high tendency to aggregate into PHFs. The abbreviation /2P stands for two isoleucine to proline mutations at positions 277 and 308 of the Tau protein sequence (I277P, I308P). These mutations inhibit the aggregation of Tau to PHFs because the prolines act as beta-sheet breakers in critical regions of the Tau molecule. The Tau construct ClaI-SalI restriction fragments were introduced into. ClaI and SalI digested pBI-L vector.

Before microinjection, the 1384 nucleotide long E. coli fragments of the pBI-5 vectors were removed by digestion with XmnI and DrdI restriction enzymes and separated on agarose gels. The linearized plasmid fragments carrying the Tau genes were microinjected into single cell embryos.

The second tTA transgene mice line (CamKIIα-tTA mice) is already available in the Lab. of Prof. H. Bujard. The tTA transgene is under the control of the calcium/calmodulin kinase IIα (CAMKIIα) promoter (Mayford et al., 1996). This tTA line allows the restricted, conditional high expression of tTA transactivator in the CNS, particularly in the hippocampus and the cortex.

Generation of Doubly Transgenic Progeny:

The Tau-BiTetO mice were crossed with CamKIIα-tTA mice to result in doubly transgenic progeny constitutively expressing both transgenes, tau construct of interest and transactivator tTA. This expression can be regulated by the presence of doxycycline, which turns off the tau gene transcription.

### Example 15

### Analysis of Transgenic Mice

Biochemical Analysis:

The inducible transgenic mice KT1/K2.1 expressing a mutant htau40/ΔK280 protein exhibits neurofibrillary tangle pathology in the cortex and in the hippocampus. FIG. 17 illustrates the biochemical analysis of neurofibrillary pathology and sarcosyl-insoluble tau in the cortex. Transgenic sarcosyl insoluble tau protein begins to accumulate in the cortex after 4 months of expression and its amount increases continuously till 8 months of age (FIG. 16b).

Histochemical Analysis of Brain Sections:

The neurofibrillary pathology in the hippocampus of the inducible transgenic mice KT1/K2.1 is illustrated with immunohistochemistry images following staining with conformational specific antibody MC1 and Alzheimer specific phospho-KXGS-tau antibody 12-E8, (FIG. 17)

Conformational- and phospho-specific tau antibodies revealed an age—related progression between 5 to 8 month of transgenic tau protein expression. Non of these antibodies bound to normal mice tau in control hippocampal sections (FIG. 17a).

### Example 16

### Generation of Inducible Mouse Neuroblastoma (N2a) Cell Lines Expressing Tau Constructs

As a basis for a cell model the 4-repeat construct K18 containing the FTDP-17 mutation ΔK280 was chosen because this has a high tendency for aggregation. Previous studies have shown that in vitro this construct K18/ΔK280 can assemble into PHFs even without the facilitation by polyanions (Barghorn et al., 2000). As a control a variant K18/ΔK280/2P containing the two point mutations I277P and T308P was chosen because these mutations interrupt beta structure and therefore prevent the aggregation of tau. N2a cell lines expressing the tau constructs K18/ΔK280 and K18/ΔK280/2P were generated using the Tet-On expression system (Urlinger et al., 2000) where protein synthesis is switched on by the addition of doxycyclin to the culture medium.

In the cell culture study, the aggregation of Tau was measured in the form of the aberrant, sarcosyl insoluble tau species which is pelletable after sarcosyl extraction (Greenberg & Davies, 1990) and can be analyzed by quantitative Western blot analysis. A pronounced aggregation of K18ΔK280. protein was found which can be seen by comparing supernatants and pellets after sarcosyl extraction (FIG. 18). The sarcosyl insoluble high-molecular-weight aggregates run as an immunoreactive smear in SDS gels (FIG. 18, lane 3).

### Example 17

### Staining of Tau Aggregates in Cells by Thioflavin-S

To confirm by an independent method whether the inducible expression of the Tau construct K18/ΔK280 in N2a cells induces aberrant aggregates indirect immunofluorescence experiments were carried out. Cells were stained with the fluorescent dye thioflavine-S (ThS), followed by staining with the polyclonal antibody K9JA that recognizes all tau isoforms independently of phosphorylation. Thioflavin-S is known as a marker of insoluble protein aggregates containing β-pleated sheets (“amyloids”). In control cells without induction of K18/ΔK280 protein, ThS-positive cells (unspecific binding) were rare (˜2%, FIG. 19). After induction of K18/ΔK280 for 3 days ThS-positive aggregates of the Tau construct were formed in 28% of the cells.

### Example 18

### Application of Inducible “Tau” Cell Line for Testing of Tau Aggregation Inhibitors

The inducible N2a cell line expressing the Tau construct K18/ΔK280 can be used for testing the inhibition of tau aggregation by low molecular weight compounds. This is illustrated in FIG. 20 for the example of emodin. In the control case K18/ΔK280 was induced in N2a cells with doxycyclin, in the test case the induction was performed in the presence of 15 μM emodin. The analysis was done by two methods:

(a) Sarcosyl extraction of cells and analysis of soluble and aggregated Tau by quantitative Western blot analysis (densitometry): FIG. 20a (lane 2) shows an example of the formation of sarcosyl insoluble high-molecular-weight aggregates of K18/ΔK280 in N2a cells not treated with emodin. They run as an immunoreactive “smear” in the SDS gel. The densitometric analysis of supernatant/pellet fractions demonstrates that 14% of the expressed K18/ΔK280 protein was found in the sarcosyl insoluble pellet (FIG. 20b). By contrast, the supernatant/pellet analysis of cells treated with 15 μM emodin (FIG. 20a, lanes 3, 4) shows that the immunoreactive smear of the pellet fraction in the SDS gel has disappeared, and significantly less material (3%) was found in the pellet fraction (FIG. 20b).

(b) Indirect immunofluorescence using ThS staining: ThS staining of N2a cells transfected with K18/ΔK280 revealed the inhibitory influence of emodin on the formation of aberrant tau aggregates. Two parallel cell cultures were incubated, one with 1 μg/ml doxycyclin (to induce the expression of the protein), another with 1 μg/ml doxycyclin and 15 μM emodin for 3 days. The quantitative analysis of N2a cells after induction of K18/ΔK280 for 3 days and staining with ThS revealed aggregates containing tau in 28% of the cells (FIG. 20c). By contrast, treatment with doxycyclin and emodin resulted in only 15% cells with ThS signal (FIG. 20c). This results indicates the inhibitory effect of emodin on tau aggregation in cell culture. An immunofluorescence image of double staining with Thioflavin-S and the tau antibody K9JA in Tet-On inducible N2a/K18/ΔK280 cells is shown in FIG. 21.

### Example 19

Selection of N2a, Tet-On, G418-Resistant Cell Line:

N2a cells were cotransfected with both the pUHD172-1 (encoding the rtTA, origin: H. Bujard Lab.) and pEU-1 (encoding G418 resistance, a derivative of pRc/CMV, Invitrogen) Plasmid DNA (20:1; 1 μg/well of 6-well plates) using the DOTAP transfection reagent (Roche). The cells were cultured in Eagle's Minimum Essential Medium (MEM) supplemented with 10% defined fetal bovine serum and subjected to G418 (600 μg/ml) and selection. The cells were fed with fresh media every 4 days for 3-4 weeks when single colonies appeared. Clones were tested for the induction level by transient transfection of pUHG 16-3 plasmid and induction of β-galactosidase was measured. The pBI-5 plasmid was also transiently transfected into these cells and the luciferase assay showed 230× induction.

Generation of Inducible Tet-On, N2a/K18/ΔK280 Cell Line:

The K18/ΔK280 DNA fragment was inserted into the bidirectional vector pBI-5 (pBI-5 is an unpublished derivative of pBI-2, Baron et al., 1995). The pBI-5/K18/ΔK280 plasmid with pX343 (a plasmid encoding the hygromycin resistance) were used for the cotransfection procedure of N2a/Tet-On, G418-resistant cells with the aid of DOTAP (20:1; 1 μg/well of 6-well plates). The cells were seeded at 4×10⁵ cells per well. On the following day cells were transferred to 100-mm dishes and selected with 100 μg/ml of hygromycin and 600 μg/ml of G418. Clonal cell line were screened for inducible K18/ΔK280 expression by measuring of luciferase activity with the luciferase assay and immunofluorescence for tau protein with the Tau antibody K9JA.

Induction of K18/ΔK280 Expression in Tet-On N2a Cells:

The inducible N2a/K18/ΔK280 cells were cultured in MEM medium supplemented with 10% fetal calf serum, 2 mM glutamine and. 0.1% nonessential amino acids. The expression of K18/ΔK280 was induced by addition of 1 μg doxycyclin per 1 ml medium. The induction was continued over 7 days and the medium was changed 3 times, always complemented with doxycyclin or with doxycyclin plus emodin.

Isolation of Soluble and Insoluble Fractions of K18/ΔK280 Protein from TetOn Inducible N2a/K18/ΔK280:

Tau Aggregation Assays:

For tau solubility assays the cells were collected by pelleting during centrifugation at 1000×g for 5 minutes. The levels and solubility of K18/ΔK280 tau protein were determined following Greenberg and Davies (1990). The cells were homogenized with Heidolph homogenizer DIAX900 in 10 vol (w/v) of buffer consisting of 10 mM Tris-HCl (pH 7.4), 0.8 M NaCl, 1 mM EGTA, and 10% sucrose. The homogenate was spun for 20 min at 20000×g, and the supernatant was retained. The pellet was rehomogenized in 5 vol of homogenization buffer and recentrifuged. Both supernatants were combined, brought to 1% N-laurylsarcosinate (w/v) and incubated for 1 hr at room temperature while shaking and centrifuged at 100 000×g for 1 hr. The sarcosyl-insoluble pellets were resuspended in 50 mM Tris-HCl (pH 7.4), 0.5 ml per g of starting material. The supernatant and sarcosyl-insoluble pellet samples were analyzed by Western blotting. The amount of material loaded for supernatant and sarcosyl insoluble pellet represented 0.75% and 15% of total material present in the supernatant and pellet respectively (the ratio of supernatant and sarcosyl-insoluble pellet was always 1:20). For quantification of the Tau level in each fraction, the Western blots were probed with antibody K9JA and analyzed by densitometry.

Quantitation of Cells with Induced Aberrant K18/ΔK280 Tau Aggregation Using ThS Staining:

Tet-On inducible N2a/K18/Δ280 cells were treated with 1 μg/ml doxycyclin for 3 days. After that the cover slips were fixed with 4% paraformaldehyde in PBS and incubated with the 0.01% ThS. Thereafter cells were washed three times in ethanol (70%). In the next step the samples were blocked with 5% BSA and treated with 0.1% Triton X-100. Finally the cells were incubated with rabbit polyclonal Tau antibody K9JA and secondary anti-rabbit antibody labeled with Cy5. Cells containing distinct ThS signals indicating the presence of insoluble aggregated material with β-pleated sheets were scored in three independent fields containing 40 cells each.

### BRIEF DESCRIPTION OF THE FIGURES

FIG. 1: Structure of inhibitor compounds, tau isoforms and constructs.

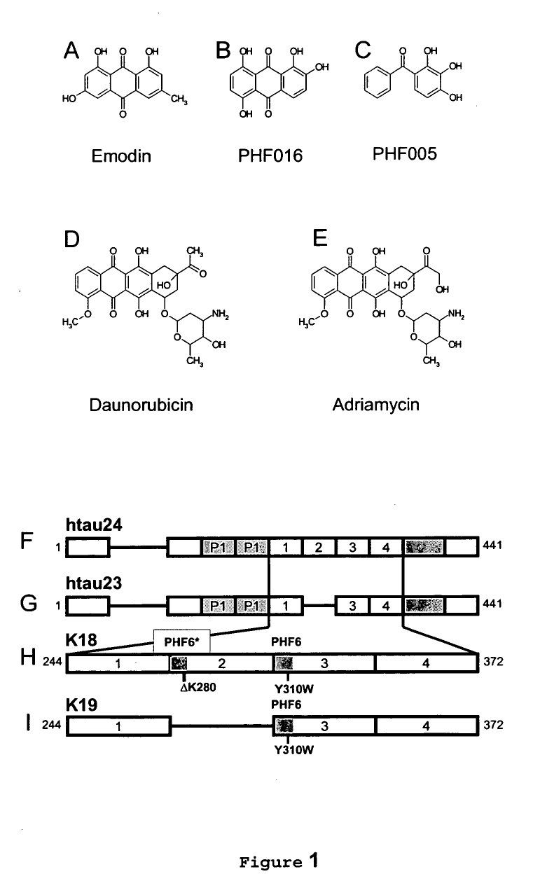

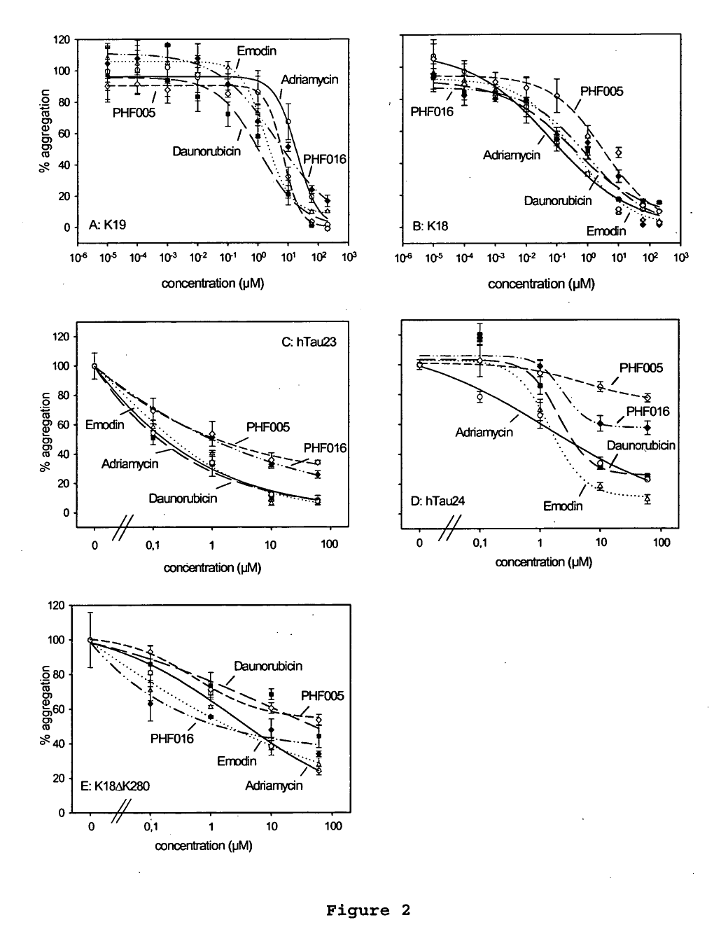

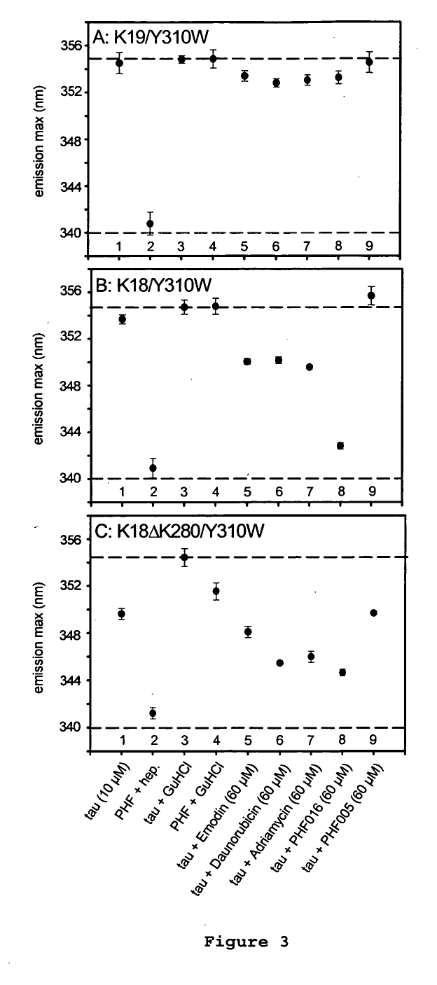

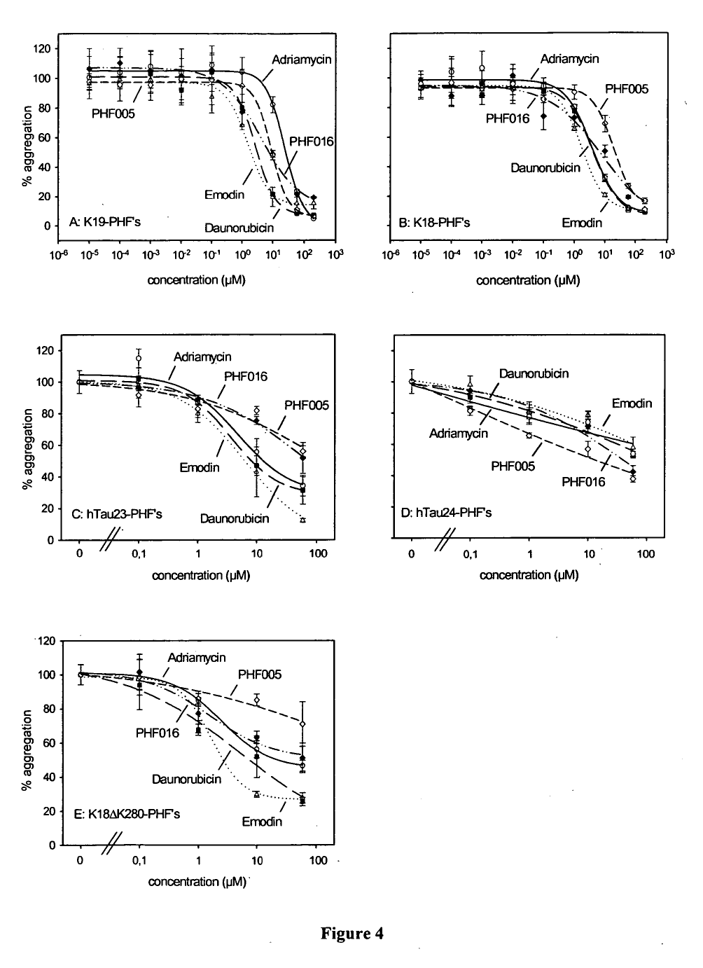

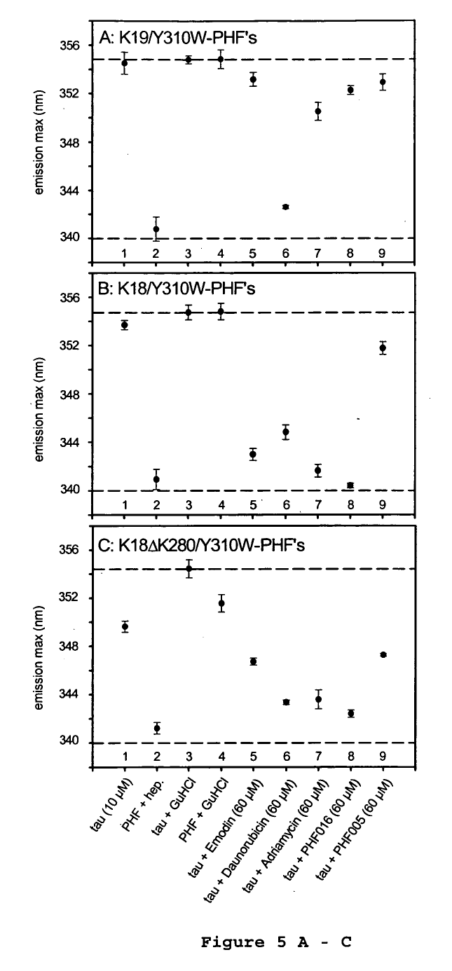

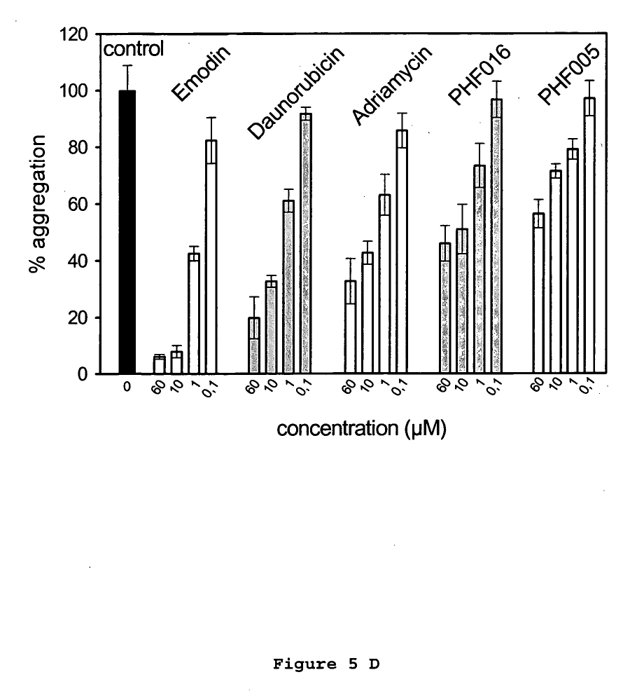

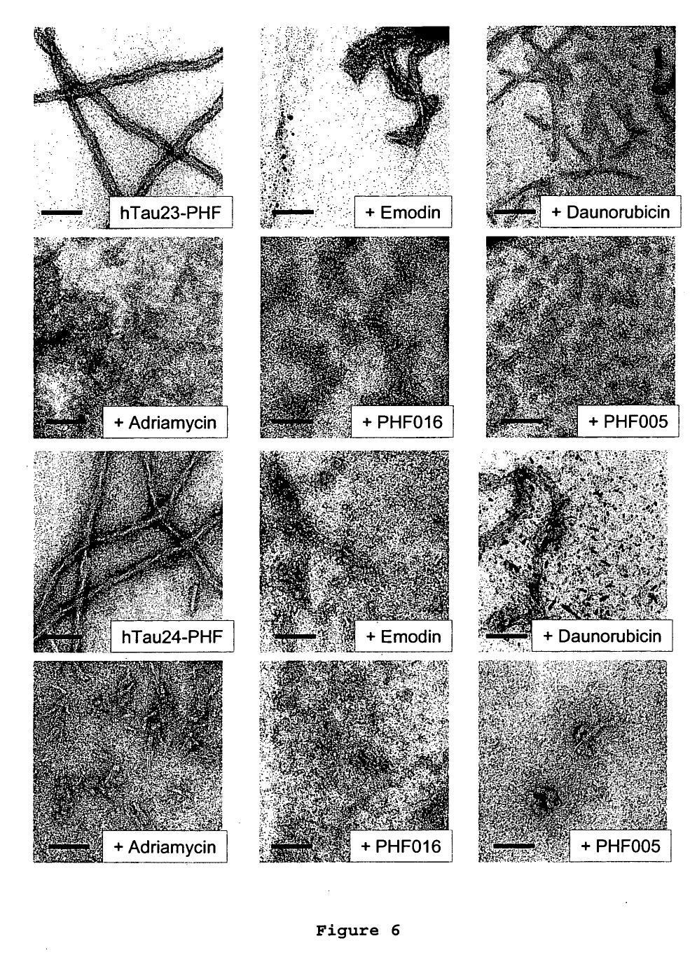

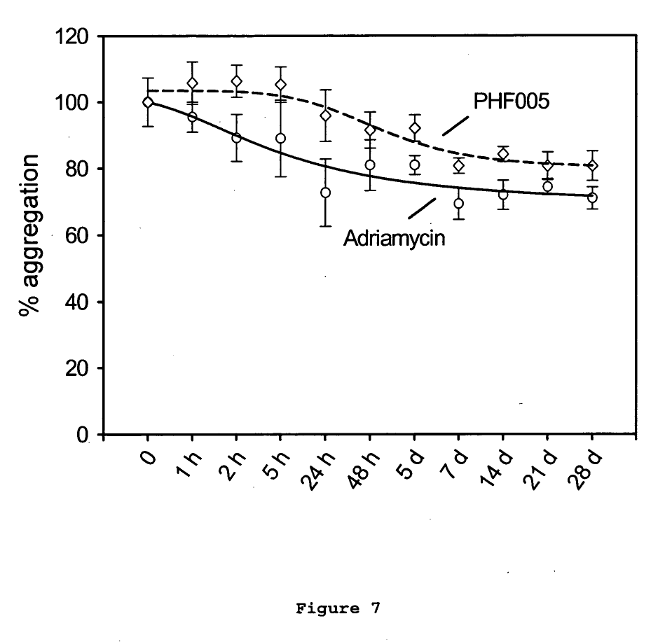

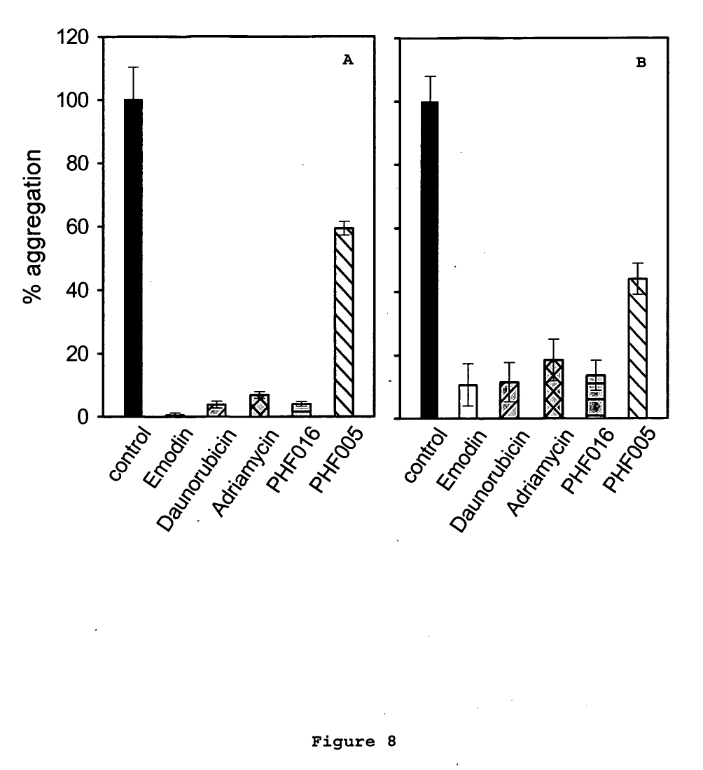

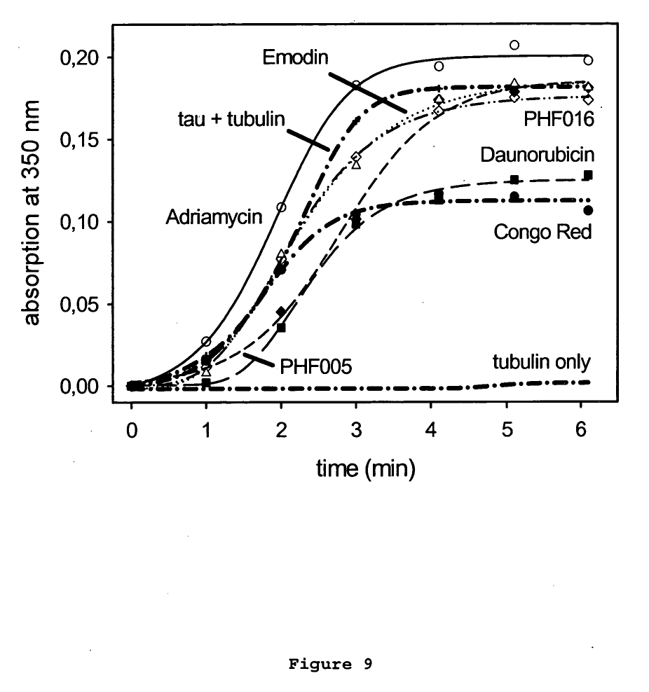

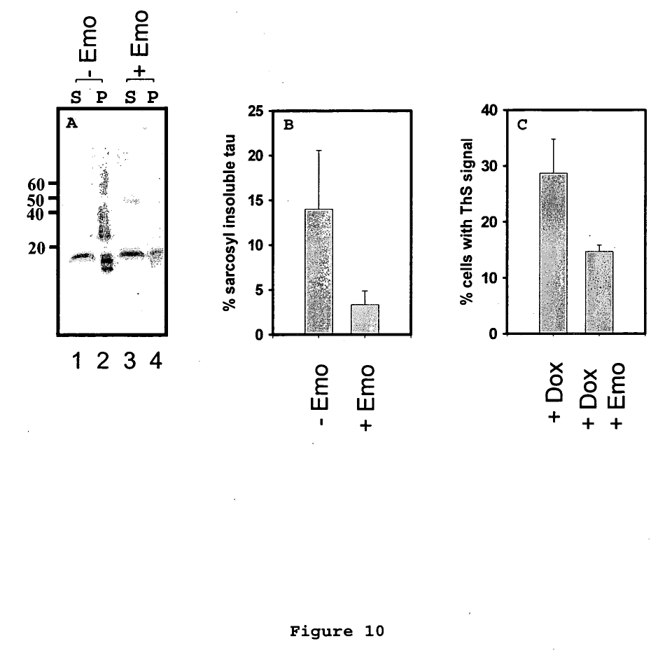

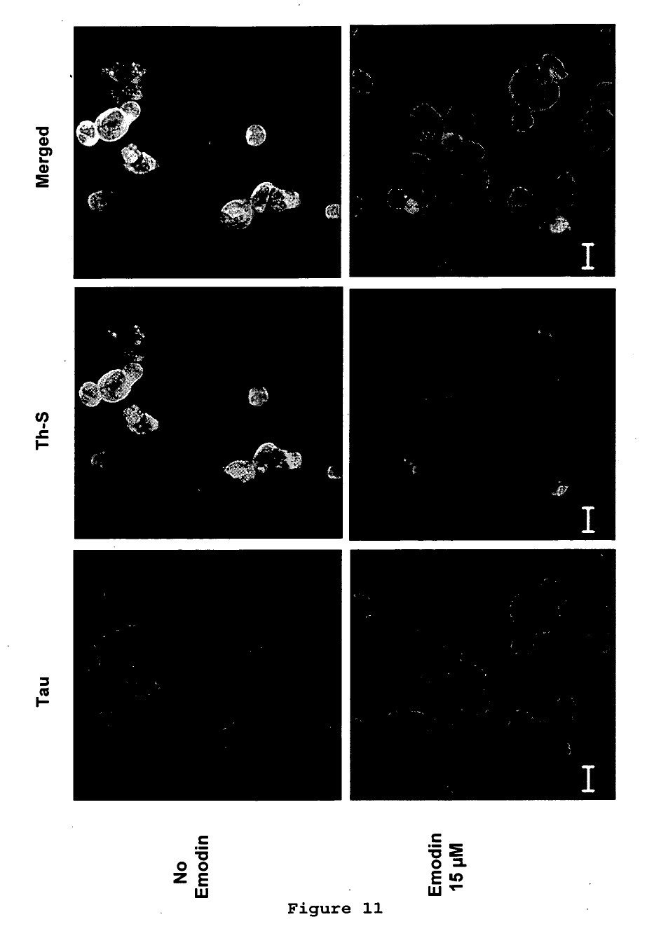

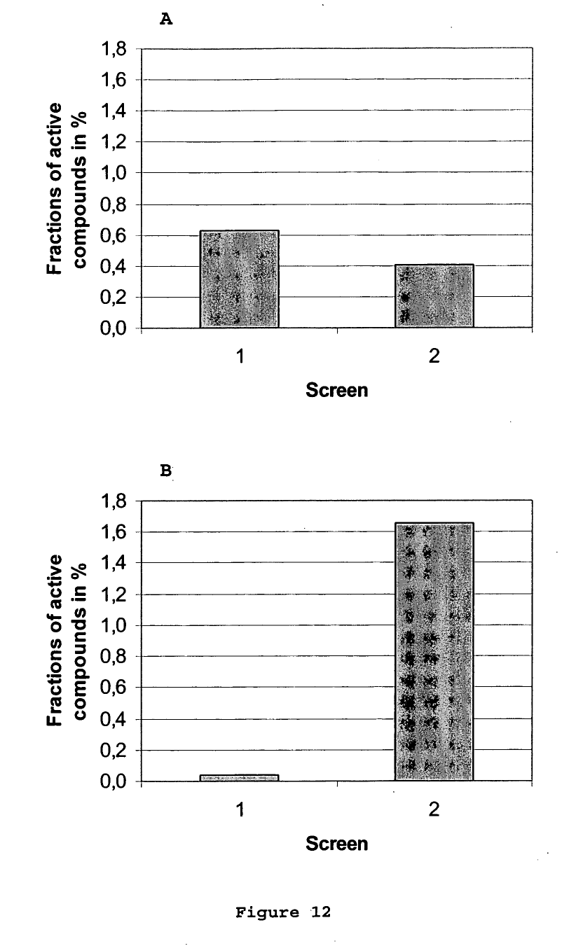

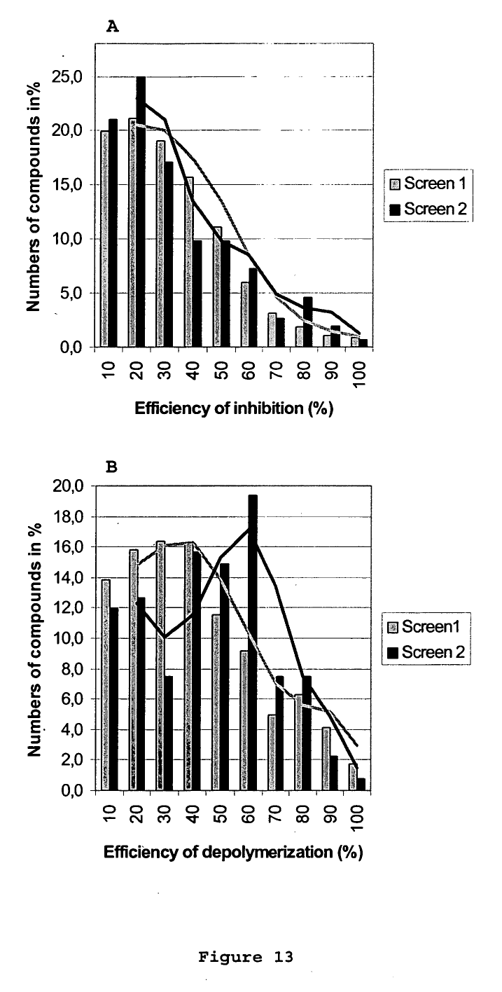

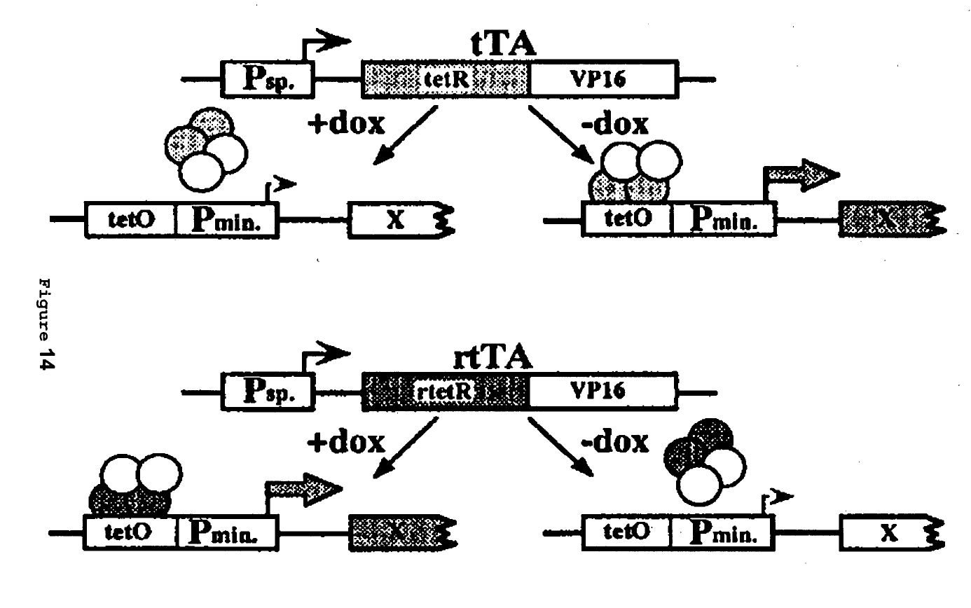

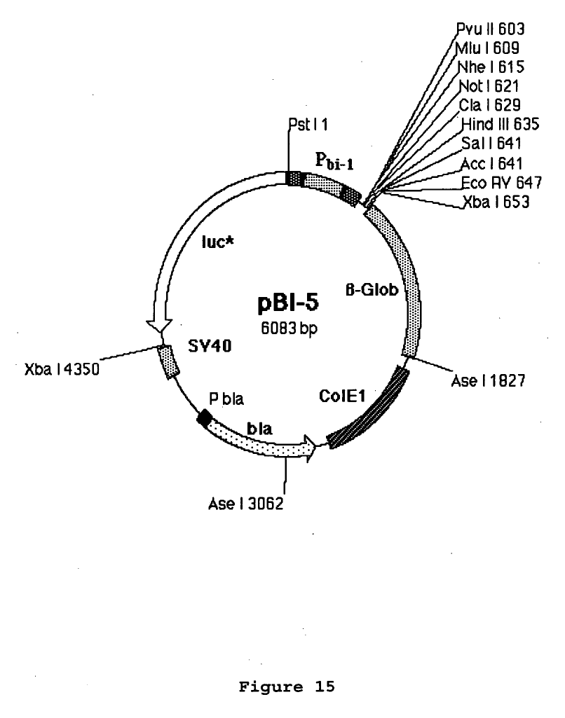

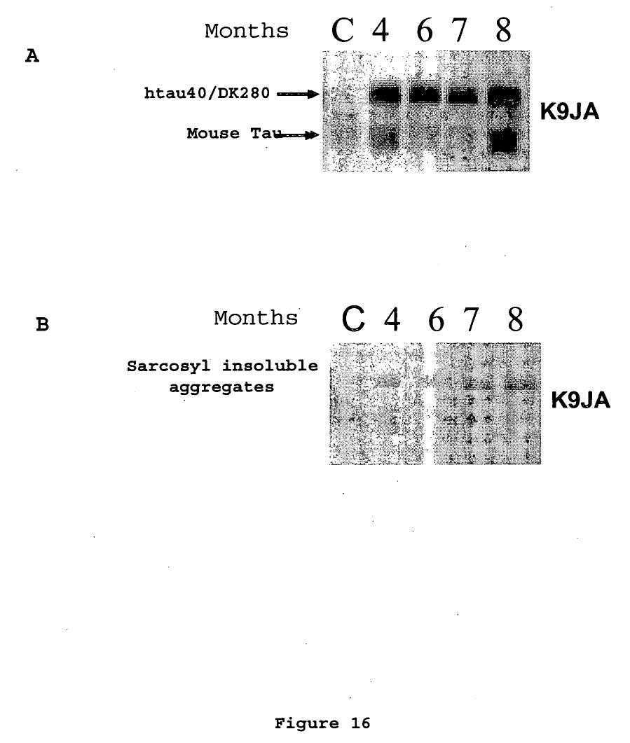

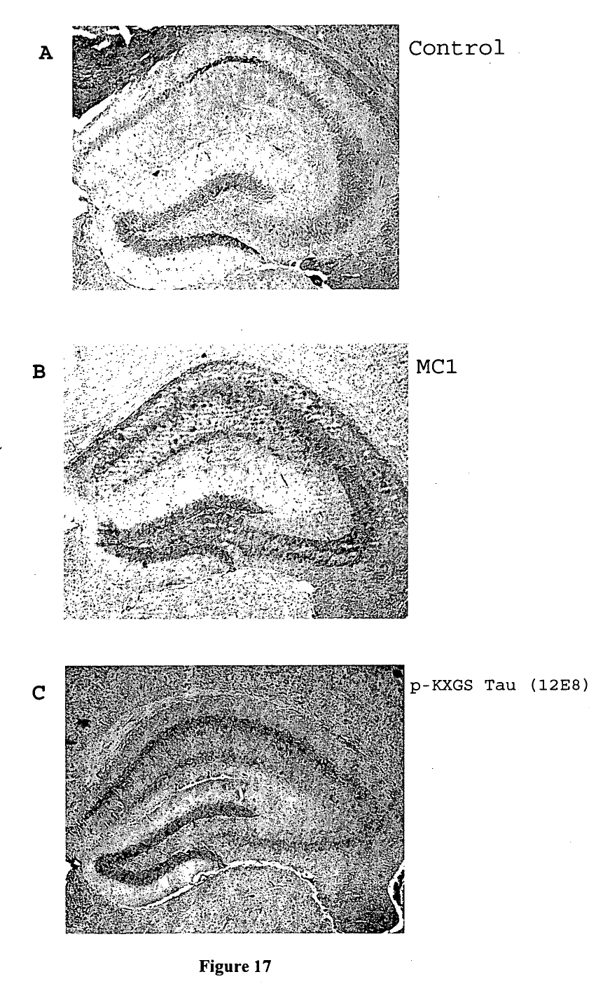

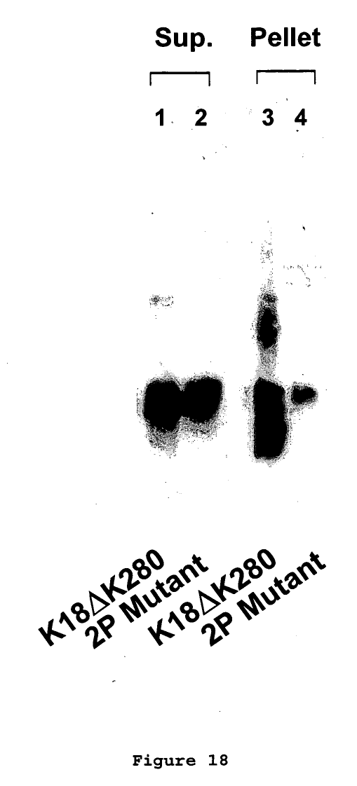

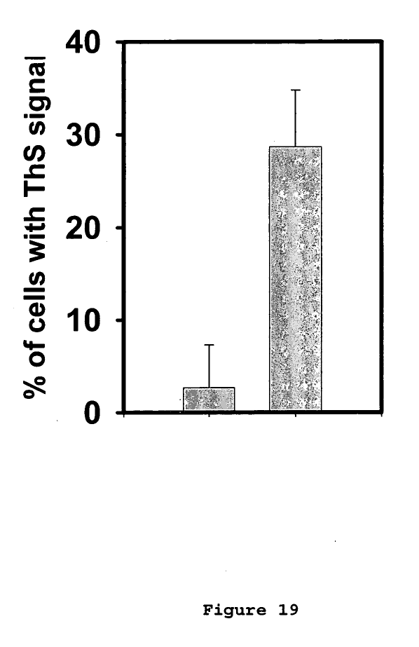

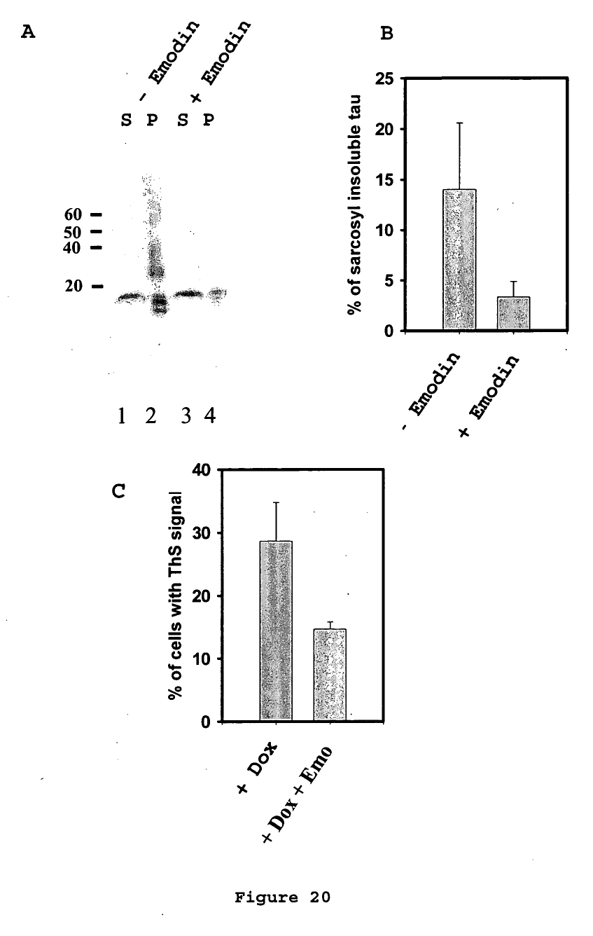

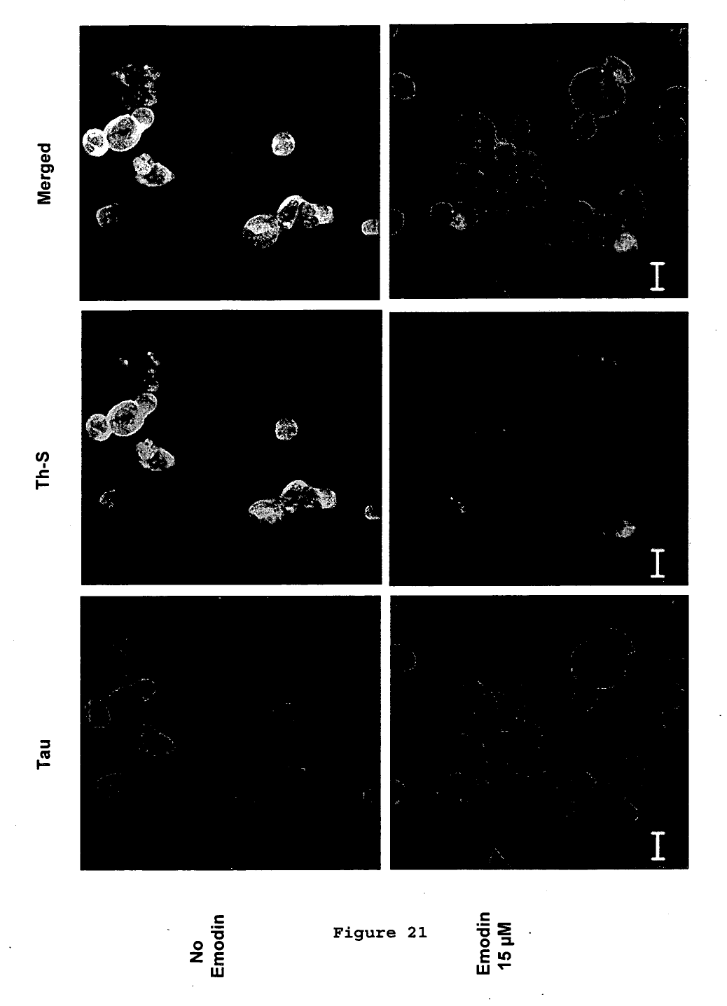

A-E, Inhibitor compounds:

(A) Emodin (1,3,8-Trihydroxy-6-methyl-anthraquinone);

(B) PHF016 (1,2,5,8-Tetrahydroxy-anthraquinone);

(C) PHF005 (1-Phenyl-1-(2,3,4-trihydroxy-phenyl)-methanone);

(D) Daunorubicin (8-Acetyl-10-(4-amino-5-hydroxy-6-methyl-tetrahydro-pyran-2-yloxy)-6,8,11-trihydroxy-1-methoxy-7,8,9,10-tetrahydro-naphthacene-5,12-dione);

(E) Adriamycin (10-(4-Amino-5-hydroxy-6-methyl-tetrahydro-pyran-2-yloxy)-6,8,11-trihydroxy-8-(2-hydroxyethanoyl)-1-methoxy-7,8,9,10-tetrahydro-naphthacene-5,12-dione;

(F-I) Tau isoforms and constructs:

(F) htau24, a four repeat isoform of tau lacking the two N-terminal inserts (numbering of the amino acids according to the longest isoform htau40);

(G) htau23, the fetal three repeat isoform lacking the two N-terminal repeats and the second repeat (exon 10);

(H) construct K18 comprising the four repeats in the microtubule binding domain;

(I) construct K19 containing three repeats. In H and I the hexapeptide motifs PHF6 (third repeat) and PHF6* (second repeat) that promote the formation of β-structure are highlighted. The position of the point mutation Y310W in the third repeat is indicated.

FIG. 2: Inhibition of PHF formation monitored by ThS-fluorescence.

(A) extent of aggregation of tau construct K19 (10 μM) plotted vs. inhibitor concentration (range 1 fM-60 μM). The extent of aggregation was measured by the thioflavin S fluorescence assay and the degree of inhibition was plotted as percentage of control. All measurements were performed in triplicates. Adriamycin (open circles, daunorubicin (filled squares), emodin (open triangles), PHF016 (filled diamonds) and PHF005 (open diamonds) exhibit only small differences over the concentration range from 10 pM to 0.1 mM. The symbols are used consistently in FIGS. 2A-E. The fits were calculated as four parameter logistic curves, the IC₅₀ values are summarised in Table 2. Half-maximal inhibition occurs in the range of 1-7 μM.

(B) inhibition of aggregation of construct K18

(C) isoform htau23

(D) isoform htau24,

(E) construct K18/ΔK280.

FIG. 3: Inhibition of PHF aggregation monitored by tryptophan fluorescence assay.

(A-C) fluorescence emission maximum of the single tryptophan W310 inserted by site-directed mutagenesis into tau constructs K19 (FIG. 3A), K18 (FIG. 3B) and K18/ΔK280 (FIG. 3C). Fully solvent-accessible Trp has an emission maximum at ˜355 nm, a blue-shift to lower wavelengths is an indicator of PHF aggregation. Soluble tau constructs (10 μM) and tau or PHFs exposed to denaturing conditions (4 M GuHCl) show the maximum of fully exposed Trp, aggregated PHFs show a maximum of 341 nm (typical of Trp buried in the interior), and tau aggregated in the presence of inhibitors (60 μM) show intermediate values, depending on the degree of inhibition. Note that by this assay, all compounds are efficient inhibitors for the aggregation of the 3-repeat construct K19, but the 4-repeat construct K18 and its mutant K18/ΔK280 mutant are much less responsive to the inhibitors.

FIG. 4: Disassembly of pre-formed PHFs induced by inhibitor compounds and monitored by ThS fluorescence.

Tau constructs and isoforms K19, K18, hTau23, hTau24 (10 μM) were first aggregated into PHFs for 48 hours in the presence of 2.5 μM heparin (except K18/ΔK280) and the polymers separated from the soluble tau by centrifugation of 1 h at 100,000 g, redissolved and then exposed to the inhibitors overnight at 37° C. at the indicated concentrations (range 0.001-200 μM). The compounds are capable of disassembling PHFs with varying efficiencies (see Table 2).

(A) construct K19, (B) K18, (C) isoform htau23, (D) isoform htau23, (E) K18/ΔK280 (no heparin). All measurements were performed in triplicates. The symbols represent adriamycin (open circles), daunorubicin (filled squares), emodin (open triangles), PHF016 (filled diamonds) and PHF005 (open diamonds).

FIG. 5: Disassembly of preformed PHFs measured by tryptophan fluorescence shift assay and filter assay. Experiments were performed with tau constructs containing the Y310W mutation as in FIG. 3.

(A) K19, (B) K18, (C) K18/ΔK280 (assembled without heparin). Note that PHF aggregation is largely reversible for K19 (except for daunorubicin), but only partially for K18 and K18/ΔK280.

(D) Depolymerisation of PHFs from htau23 measured by filter assay. The bars show the fraction of polymerised material trapped on the PVDF membrane. Black bar =control, untreated PHFs. The groups of bars show disassembly by emodin, daunorubicin, adriamycin, PHF016, PHF005 as a function of compound concentration.

FIG. 6: Electron microscopy of inhibited and disassembled PHFs.

FIG. 7: Time course of PHF disassembly at low inhibitor concentrations.

PHFs were formed as above (see FIG. 4; 10 μM construct K19, 2.5 μM heparin, overnight) and then exposed to 0.5 μM adriamycin or PHF005. Note that in spite of the low inhibitor concentrations there is a gradual decrease of PHFs. Untreated controls were measured in parallel and subtracted as background.

FIG. 8: Effect of PHF inhibitors on Aβ fibre aggregation and disassembly.

Aβ peptide 1-40 (10 μM) was incubated with moderate shaking overnight at room temperature and incubated with various compounds (60 μM) overnight.

(A) inhibition of fibre aggregation is most efficient in the case of emodin, daunorubicin, and PHF0016.

(B) disassembly of pre-formed fibrils.

FIG. 9: Effect of compounds on microtubule binding 30 μM tubulin dimer was incubated in a microtiter plate at 37° C. in the absence and presence of htau40 (10 μM) and 60 μM compound. Absorbance was taken at 350 nm and plotted versus time. The symbols refer to adriamycin (open circles), daunorubicin (filled squares), emodin (open triangles), PHF016 (filled diamonds) and PHF005 (open diamonds). All curves (except tubulin only) show microtubule assembly within a few minutes.

FIG. 10: Effect of the aggregation inhibitor emodin on tau aggregation in cells.

(A) Western blotting of fractionated lysates from inducible N2a cells expressing tau (K18/ΔK280) after sarkosyl extraction. Sarcosyl insoluble K18/ΔK280 tau was detected in these cells after 7 days of induction. The sarcosyl-soluble (S) and -insoluble pellet fractions (P) were separated by high speed centrifugation. The pellets obtained from cells incubated without (−) and with 15 μM emodin (+) were resuspended in Tris-EDTA buffer in a volume equivalent to 5% of the extracts. Note that the amount of material loaded for supernatant and pellet represents 1% and 20% of the total-extracted material, respectively.

(B) Histogram of sarcosyl insoluble tau (K18/ΔK280) from cells grown without emodin or with 15 μM emodin (see FIG. 10A, lanes 2, 4).

(C) Histogram of number of N2a cells expressing K18/ΔK280 (after induction with doxycyclin) with distinct thioflavine S signal in cell cultures induced without emodin (+Dox) or with 15 μM Emodin (+Dox, +Emo). Note that emodin inhibits the aggregation about 2-fold as measured by ThS.

FIG. 11: Tau expression and aggregation in N2a cells.

N2a cells were induced to express K18/ΔK280 and fixed after 3 days. They were sequentially double stained with Thioflavin-S (green) and the pan-tau antibody K9JA (red).

Top row: without emodin, bottom row: with 15 μM emodin. Left: immunofluorescence with tau antibody, middle: ThS staining, right: merge. Note the reduced ThS staining of cells in the presence of 15 μM emodin (middle, top and bottom).

FIG. 12: Fractions of inhibiting and depolymerising compounds in the first and second screen.

(A) Fractions of compounds which exhibited an inhibitory effect >90% at 60 μM concentration.

(B) Fractions of depolymerising compounds with an activity >80%.

FIG. 13: Histograms of the activity of compounds in terms of inhibition and reversal of PHF formation

(A) The distribution of compounds in percent is plotted against their efficiency to inhibit PHF assembly at a concentration of 60 μM. For both the first'screen (200.000 compounds, blue bars) and the second screen (175 compounds, red bars) a peak at 10-20% efficiency appears, i.e. a large number of compounds has a mild effect, but only few reach an efficiency close to 100%.

(B) Distribution of compounds plotted against their efficiency of depolymerising pre-formed PHFs. Note the difference between the first screen (blue bars) and the second screen (red bars). The compounds from the first screen show a peak at 30-40% efficiency, whereas the compounds of the second screen exhibit a maximum at 60-70%, indicating that the average efficiency has been improved.

FIG. 14: tTA and rtTA tetracycline gene regulation system

tTA is a fusion protein composed of the repressor (tetR) of the Tn10 Tc-resistance operon of Escherichia coli and a C-terminal portion of protein 16 of herpes simplex virus that functions as strong transcription activator. tTA binds in the absence of doxycyclin (but not in its presence) to an array of seven cognate operator sequences (tetO) and activates transcription from a minimal human cytomegalovirus (hCMV) promoter, which itself is inactive.

FIG. 15: pBI-5 plasmid map

The pBI-5 plasmid was originally constructed in H. Bujard's laboratory (Baron et al., 1995), but is now available from Clontech under the name pBI-L. The bidirectional Tet vectors are used to simultaneously express two genes under the control of a single TRE (tetracycline-responsive element) consisting of seven direct repeats of a 42-bp sequence containing the tetO (tetracycline operator) followed downstream and upstream by the minimal CMV promoter (P_{minCMV}). pBI-L can be used to indirectly monitor the expression of tau protein by following the activity of the reporter, gene luciferase expressed at the same time downstream of TRE.

FIG. 16: Analysis of neurofibrillary pathology and sarcosyl-in-soluble tau in the cortex of the inducible transgenic mice KT1/K2.1

(A) The phosphorylation independent tau-antibody K9JA shows the expression of htau40/ΔK280 in the brains of transgenic mice after induction between 4 and 8 months.

(B) The phosphorylation independent tau-antibody K9JA shows the transgenic sarcosyl insoluble htau40/ΔK280 protein. Aggregation of the protein begins in cortex after 4 months of induction.

FIG. 17: Histochemical analysis of brain sections

Low magnification views of the hippocampus showing:

(A) control mouse,

(B) transgenic mouse expressing human tau40/ΔK280 in pyramidal neurons which are immunostained by the antibody MC1 which recognizes an Alzheimer like conformation of tau

(C) human tau40/ΔK280 immunopositive pyramidal neurons following staining with phospho-tau antibody 12E8, which detects phosphorylated tau protein at the KXGS motifs in the repeats (Ser262 and Ser356).

FIG. 18: Aggregation of K18/ΔK280 protein in N2a cells after 5 days of induction of K18/ΔK280 by doxycycline

Blots comparing supernatants (lanes 1, 2) and pellets (lane 3, 4) after sarcosyl extraction of tau. The expression of K18/ΔK280 leads to the formation of sarcosyl insoluble high-molecular-weight aggregates which run as an immunoreactive smear in SDS gels (lane 3).

By contrast, only a small amount of the double proline mutant of K18/ΔK280/2P was found in the sarcosyl insoluble pellet (lane 4).

FIG. 19: Thioflavin-S positive N2a cells without and after induction of K18/ΔK280 with doxycylin

In control cells without induction of K18/ΔK280 protein, cells positive for Thioflavin-S (unspecific binding) are rare (˜2%). After induction of K18/ΔK280 for 3 days ThS positive aggregates are formed in 28% of the cells.

FIG. 20: Analysis of Tau aggregation

(A) Western blotting of fractionated lysates obtained from inducible N2a/K18/ΔK280 cells after sarcosyl extraction. Sarcosyl-insoluble Tau was detected after 7 days of induction. The sarcosyl-soluble (S) and -insoluble pellet (P) fractions were separated by centrifugation at high speed. The pellets obtained from cells incubated without (−) and in the presence of 15 μM Emodin (+) were resuspended in TE buffer at a volume equivalent to 5% of the extracts. Note that the amount of material loaded for supernatant and pellet represented 1% and 20% of the total material extracted, respectively.

(B) Histogram of the sarcosyl insoluble K18/ΔK280 protein fraction obtained from cells grown without emodin (compare FIG. 20A, lane 2) and in the presence of 15 μM emodin (compare FIG. 20A, lane 4).

(C) Histogram of the number of inducible N2a/K18/ΔK280 cells with distinct thioflavine S signal in cell cultures induced in the absence of emodin (+Dox) and induced in the presence of 15 μM emodin (+Dox, +Emo).

FIG. 21: Immunofluorescence imaging of Tau aggregates in cells

Double staining with Thioflavin-S and Tau antibody K9JA in Tet-On inducible N2a/K18/ΔK280 cells. The cells were fixed 3 days post induction and sequentially double stained with Thioflavin-S (green) and tau antibody K9JA. The staining ThS intensities of cells induced with doxycyclin in the presence of 15 μM emodin are distinctly lower than in cells induced without emodin (compare the quantitative analysis in FIG. 20C).

### REFERENCES

Ball, M. J., and Murdoch, G. H.(1997). Neuropathological criteria for the diagnosis of Alzheimer's disease: are we really ready yet? Neurobiol Aging 18, S3-12.

Barghorn, S., Zheng-Fischhofer, Q., Ackmann, M., Biernat, J., von Bergen, M., Mandelkow, E. M., and Mandelkow, E. (2000). Structure, microtubule interactions, and paired helical filament aggregation by tau mutants of frontotemporal dementias. Biochemistry 39, 11714-11721.

Barghorn S, Mandelkow E. (2002) Toward a unified scheme for the aggregation of tau into Alzheimer paired helical filaments. Biochemistry 41(50):14885-96.

Bates, G. (2003). Huntingtin aggregation and toxicity in Huntington's disease. Lancet 361, 1642-1644.

Beirao, I., Lobato, L., Riedstra, S., Costa, P. M., Pimentel, S., and Guimaraes, S. (1999). Ineffectiveness of dialysis in transthyretin (TTR) clearance in familial amyloid polyneuropathy type I, in spite of lower stability of the TTR Met30 variant. Clin Nephrol 51, 45-49.

Bonifacio, M. J., Sakaki, Y., and Saraiva, M. J. (1996). ‘In vitro’ amyloid fibril formation from transthyretin: the influence of ions and the amyloidogenicity of TTR variants. Biochim Biophys Acta 1316, 35-42.

Braak, H., and Braak, E. (1991). Neuropathological stageing of Alzheimer-related changes. Acta Neuropathol (Berl) 82, 239-259.

Brion, J. P., Anderton, B. H., Authelet, M., Dayanandan, R., Leroy, K., Lovestone, S., Octave, J. N., Pradier, L., Touchet, N., and Tremp, G. (2001). Neurofibrillary tangles and tau phosphorylation. Biochem Soc Symp, 81-88.

Buee L, Hamdane M, Delobel P, Sambo A V, Begard S, Ghestem A, Sergeant N, Delacourte A. (2002) [Tau story: from frontotemporal dementia to other tauopathies]. J Soc Biol 196(1):103-8.

Caughey, B., and Lansbury, P. T., Jr. (2003). Protofibrils, Pores, Fibrils, and Neurodegeneration: Separating the Responsible Protein Aggregates from the Innocent Bystanders. Annu Rev Neurosci.

Dou, F., Netzer, W. J., Tanemura, K., Li, F., Hartl, F. U., Takashima, A., Gouras, G. K., Greengard, P., and Xu, H. (2003). Chaperones increase association of tau protein with microtubules. Proc Natl Acad Sci USA 100, 721-726.

Duff, K., Knight, H., Refolo, L. M., Sanders, S., Yu, X., Picciano, M., malester, B., Hutton, M., Adamson, J., Goedert, M., et al. (2000). Characterization of pathology in transgenic mice over-expressing human genomic and cDNA tau transgenes. Neurobiol Dis 7, 87-98.

Eidenmuller, J., Fath, T., Maas, T., Pool, M., Sontag, E., and Brandt, R. (2001). Phosphorylation-mimicking glutamate clusters in the proline-rich region are sufficient to simulate the functional deficiencies of hyperphosphorylated tau protein. Biochem J 357, 759-767.

Friedhoff, P., Schneider, A., Mandelkow, E. M., and Mandelkow, E. (1998a). Rapid assembly of Alzheimer-like paired helical filaments from microtubule-associated protein tau monitored by fluorescence in solution. Biochemistry 37, 10223-10230.

Friedhoff, P., von Bergen, M., Mandelkow, E. M., Davies, P., and Mandelkow, E. (1998b). A nucleated assembly mechanism of Alzheimer paired helical filaments. Proc Natl Acad Sci USA 95, 15712-15717.

Glabe, C. (2001). Intracellular mechanisms of amyloid accumulation and pathogenesis in Alzheimer's disease. J Mol Neurosci 17, 137-145.

Goedert, M., Jakes, R., Spillantini, M. G., Hasegawa, M., Smith, M. J., and Crowther, R. A. (1996). Assembly of microtubule-associated protein tau into Alzheimer-like filaments induced by sulphated glycosaminoglycans. Nature 383, 550-553.

Goedert, M., Spillantini, M. G., and Davies, S. W. (1998). Filamentous nerve cell inclusions in neurodegenerative diseases. Curr Opin Neurobiol 8, 619-632.

Goldberg, M. S., and Lansbury, P. T., Jr. (2000). Is there a cause-and-effect relationship between alpha-synuclein fibrillization and Parkinson's disease? Nat Cell Biol 2, E115-119.

Gotz, J. (2001). Tau and transgenic animal models. Brain Res Brain Res Rev 35, 266-286.

Hall, G. F., Chu, B., Lee, G., and Yao, J. (2000). Human tau filaments induce microtubule and synapse loss in an in vivo model of neurofibrillary degenerative disease. J Cell Sci 113 (Pt 8), 1373-1387.

Heiser, V., Scherzinger, E., Boeddrich, A., Nordhoff, E., Lurz, R., Schugardt, N., Lehrach, H., and Wanker, E. E. (2000). Inhibition of huntingtin fibrillogenesis by specific antibodies and small molecules: implications for Huntington's disease therapy. Proc Natl Acad Sci USA 97, 6739-6744.

Higuchi, M., Ishihara, T., Zhang, B., Hong, M., Andreadis, A., Trojanowski, J., and Lee, V. M. (2002a). Transgenic mouse model of tauopathies with glial pathology and nervous system degeneration. Neuron 35, 433-446.

Higuchi, M., Lee, V. M., and Trojanowski, J. Q. (2002b). Tau and axonopathy in neurodegenerative disorders. Neuromolecular Med 2, 131-150.

Hutton, M. (2001). Missense and splice site mutations in tau associated with FTDP-17: multiple pathogenic mechanisms. Neurology 56, S21-25.

Hutton, M., Lewis, J., Dickson, D., Yen, S. H., and McGowan, E. (2001). Analysis of tauopathies with transgenic mice. Trends Mol Med 7, 467-470.

Iqbal, K. & Grundke-Iqubal, I. (1997). Elevated levels of τ and ubiquitin in brain and cerebrospinal fluid in Alzheimer's disease. Int. Psychogeriatr. 9 (Suppl. 1), 289-296

Jackson, G. R., Wiedau-Pazos, M., Sang, T. K., Wagle, N., Brown, C. A., Massachi, S., and Geschwind, D. H. (2002). Human wild-type tau interacts with wingless pathway components and produces neurofibrillary pathology in Drosophila. Neuron 34, 509-519.

Johnson, G. V., and Bailey, C. D. (2002). Tau, where are we now? J Alzheimers Dis 4, 375-398.

Kraemer, B. C., Zhang, B., Leverenz, J. B., Thomas, J. H., Trojanowski, J. Q., and Schellenberg, G. D. (2003). Neurodegeneration and defective neurotransmission in a Caenorhabditis elegans model of tauopathy. Proc Natl Acad Sci USA 100, 9980-9985.

Lambert, M. P., Viola, K. L., Chromy, B. A., Chang, L., Morgan, T. E., Yu, J., Venton, D. L., Krafft, G. A., Finch, C. E., and Klein, W. L. (2001). Vaccination with soluble Abeta oligomers generates toxicity-neutralizing antibodies. J Neurochem 79, 595-605.

Lee, V. M., Goedert, M., and Trojanowski, J. Q. (2001). Neurodegenerative tauopathies. Annu Rev Neurosci 24, 1121-1159.

Lewis, J. McGowan, E., Rockwood, J., Melrose, H., Nacharaju, P., Van Slegtenhorst, M., Gwinn-Hardy, K., Paul Murphy, M., Baker, M., Yu, X., et al. (2000). Neurofibrillary tangles, amyotrophy and progressive motor disturbance in mice expressing mutant (P301L) tau protein. Nat Genet 25, 402-405.

Li, L., von Bergen, M., Mandelkow, E. M., and Mandelkow, E. (2002). Structure, stability, and aggregation of paired helical filaments from tau protein and FTDP-17 mutants probed by tryptophan scanning mutagenesis. J Biol Chem 277, 41390-41400.

Liu, F., Iqbal, K., Grundke-Iqbal, I., and Gong, C. X. (2002). Involvement of aberrant glycosylation in phosphorylation of tau by cdk5 and GSK-3beta. FEBS Lett 530, 209-214.

Maccioni, R. B., Otth, C., Concha, II, and Munoz, J. P. (2001). The protein kinase Cdk5. Structural aspects, roles in neurogenesis and involvement in Alzheimer's pathology. Eur J Biochem 268, 1518-1527.

Perez, M., Valpuesta, J. M., Medina, M., Montejo de Garcini, E., and Avila, J. (1996). Polymerization of tau into filaments in the presence of heparin: the minimal sequence required for tau-tau interaction. J Neurochem 67, 1183-1190.

Podlisny M B, Walsh D M, Amarante P, Ostaszewski B L, Stimson E R, Maggio J E, Teplow D B, Selkoe D J. (1998) Oligomerization of endogenous and synthetic amyloid beta-protein at nanomolar levels in cell culture and stabilization of monomer by Congo red. Biochemistry 37(11):3602-11.

Rochet, J. C., and Lansbury, P. T., Jr. (2000). Amyloid fibrillogenesis: themes and variations. Curr Opin Struct Biol 10, 60-68.

Sanchez, I., Mahlke, C., and Yuan, J. (2003). Pivotal role of oligomerization in expanded polyglutamine neurodegenerative disorders. Nature 421, 373-379.

Schenk, D., Barbour, R., Dunn, W., Gordon, G., Grajeda, H., Guido, T., Hu, K., Huang, J., Johnson-Wood, K., Khan, K., et al. (1999). Immunization with amyloid-beta attenuates Alzheimer-disease-like pathology in the PDAPP mouse. Nature 400, 173-177.

Schneider,. A., Biernat, J., von Bergen, M., Mandelkow, E., and Mandelkow, E. M. (1999). Phosphorylation that detaches tau protein from microtubules (Ser262, Ser214) also protects it against aggregation into Alzheimer paired helical filaments. Biochemistry 38, 3549-3558.

Selkoe, D. J. (1989). The deposition of amyloid proteins in the aging mammalian brain: implications for Alzheimer's disease. Ann Med 21, 73-76.

Sheleg, S. V., Korotkevich, E. A., Zhavrid, E. A., Muravskaya, G. V., Smeyanovich, A. F., Shanko, Y. G., Yurkshtovich, T. L., Bychkovsky, B. and Belyaev, S. A. (2002). Local chemotherapy with cisplatin-depot for glioblastoma multiforme. J. Neurooncol. 60(1)., 53-59

Shtilerman, M. D., Ding, T. T., and Lansbury, P. T., Jr. (2002). Molecular crowding accelerates fibrillization of alpha-synuclein: could an increase in the cytoplasmic protein concentration induce Parkinson's disease? Biochemistry 41, 3855-3860.

Schweers, O., Mandelkow, E. M., Biernat, J., and Mandelkow, E. (1995). Oxidation of cysteine-322 in the repeat domain of microtubule-associated protein tau controls the in vitro assembly of paired helical filaments. Proc Natl Acad Sci USA 92, 8463-8467.

Selkoe D J, Schenk D. Alzheimer's disease: molecular understanding predicts amyloid-based therapeutics. (2003) Annu Rev Pharmacol Toxicol 43:545-84.

van Swieten, J. C., Stevens, M., Rosso, S. M.,. Rizzu, P., Joosse, M., de Koning, I., Kamphorst, W., Ravid, R., Spillantini, M. G., Niermeijer, and Heutink, P. (1999). Phenotypic variation in hereditary frontotemporal dementia with tau mutations. Ann Neurol 46, 617-626.

Volles, M. J., and Lansbury, P. T., Jr. (2003). Zeroing in on the pathogenic form of alpha-synuclein and its mechanism of neurotoxicity in Parkinson's disease. Biochemistry 42, 7871-7878.

Von Bergen M, Friedhoff P, Biernat J, Heberle J. Mandelkow E M, Mandelkow E. (2000) Assembly of tau protein into Alzheimer paired helical filaments depends on a local sequence motif ((306)VQIVYK(311)) forming beta structure. Proc Natl Acad Sci USA 97(10):5129-34.

von Bergen, M., Barghorn, S., Li, L., Marx, A., Biernat, J., Mandelkow, E. M., and Mandelkow, E. (2001). Mutations of tau protein in frontotemporal dementia promote aggregation of paired helical filaments by enhancing local beta-structure. J Biol Chem 276, 48165-48174.

Wille, H., Drewes, G., Biernat, J., Mandelkow, E. M., and Mandelkow, E. (1992). Alzheimer-like paired helical filaments and antiparallel dimers formed from microtubule-associated protein tau in vitro. J Cell Biol 118, 573-584.

Wischik, C. M., Edwards, P. C., Lai, R. Y., Roth, M., and Harrington, C. R. (1996). Selective inhibition of Alzheimer disease-like tau aggregation by phenothiazines. Proc Natl Acad Sci USA 93, 11213-11218.

Wittmann, C. W., Wszolek, M. F., Shulman, J. M., Salvaterra, P. M., Lewis, J., Hutton, M., and Feany, M. B. (2001). Tauopathy in Drosophila: neurodegeneration without neurofibrillary tangles. Science 293, 711-714.

Zhang, J., and Johnson, G. V. (2000). Tau protein is hyperphosphorylated in a site-specific manner in apoptotic neuronal PC12 cells. J Neurochem 75, 2346-2357.

Baron U, Freundlieb S, Gossen M, Bujard H (1995). Coregulation of two gene activities by tetracycline via a bidirectional promoter. Nucleic Acids Res. (17):3605-3606.

Furth, P. A., St. Onge, L., Boger, H., Gruss, P., Gossen, M., Kistner, A., Bujard, H., Hennighausen, L. (1994) Temporal control of gene expression in transgenic mice by a tetracycline-responsive promoter. Proc Natl Acad Sci USA, 91:9302-9306.

Gossen M, Freundlieb S, Bender G, Muller G, Hillen W, Bujard H. (1995). Transcriptional activation by tetracyclines in mammalian cells. Science 268:1766-1769.

Gossen & Bujard (2002). Studying gene function in eukaryotes by conditional gene inactivation. Annu Rev Genet. 36:153-73

Greenberg S G, Davies P (1990). A preparation of Alzheimer paired helical filaments that displays distinct tau proteins by polyacrylamide gel electrophoresis. Proc Natl Acad Sci USA. 87, 5827-5831.

Mayford, M., Bach, M. E., Huang, Y. Y., Wang, L., Hawkins, R. D., Kandel, E. R. (1996). Control of memory formation through regulated expression of a CaMKII transgene. Science, 274:1678-1683.

Rizzu P, Van Swieten J C, Joosse M, Hasegawa M, Stevens M, Tibben A, Niermeijer M F, Hillebrand M, Ravid R, Oostra B A, Goedert M, van Duijn C M, Heutink P. (1999)

High prevalence of mutations in the microtubule-associated protein tau in a population study of frontotemporal dementia in the Netherlands.

Am J Hum Genet. 64, 14-21.

Urlinger S., Baron U, Thellmann M, Hasan M T, Bujard H, Hillen W (2000). Exploring the sequence space for tetracycline-dependent transcriptional activators: novel mutations yield expanded range and sensitivity. Proc Natl Acad Sci USA. 97:7963-7968.

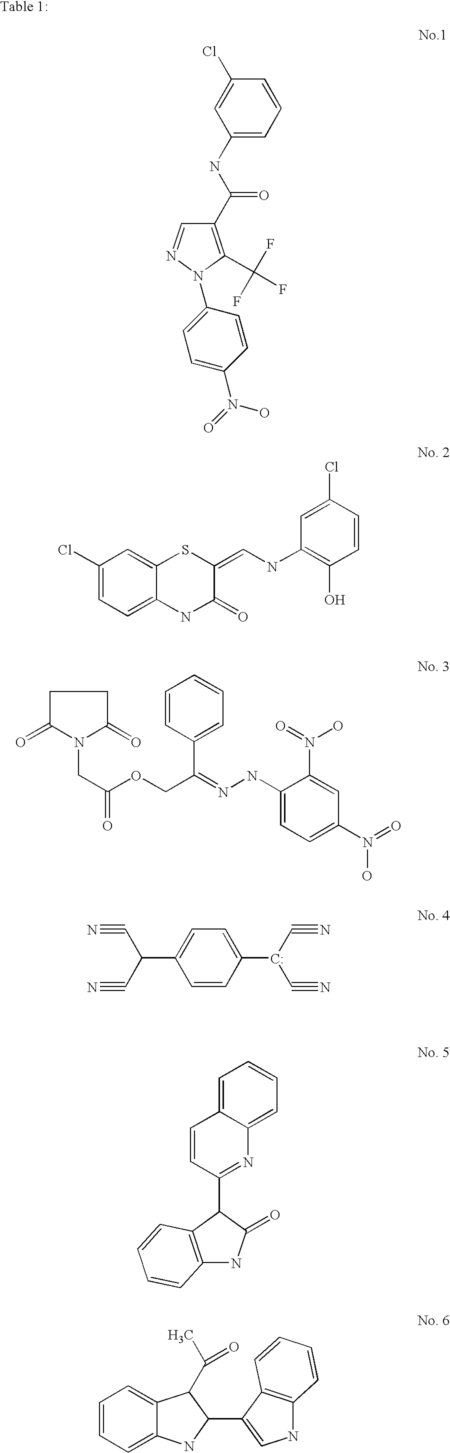

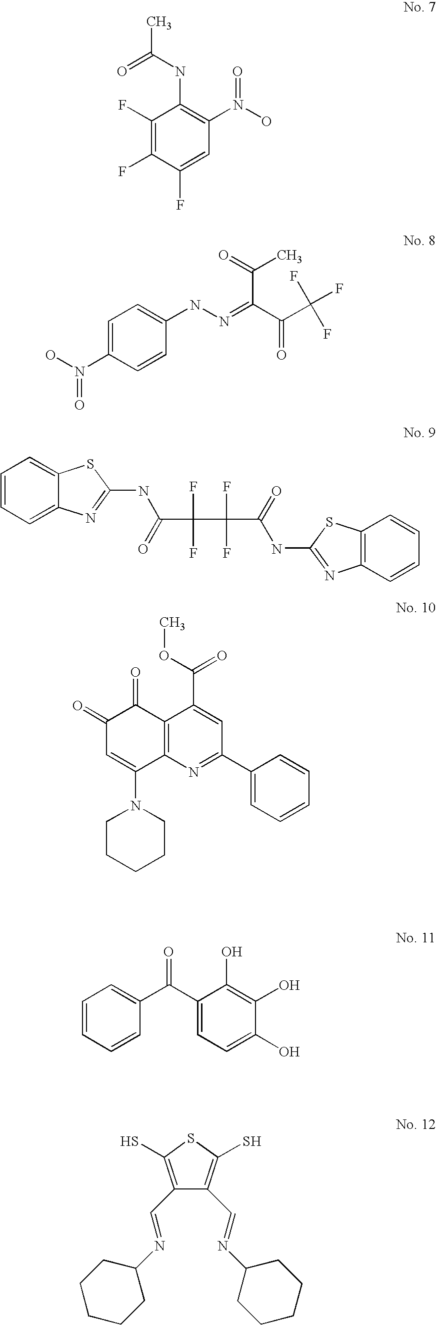

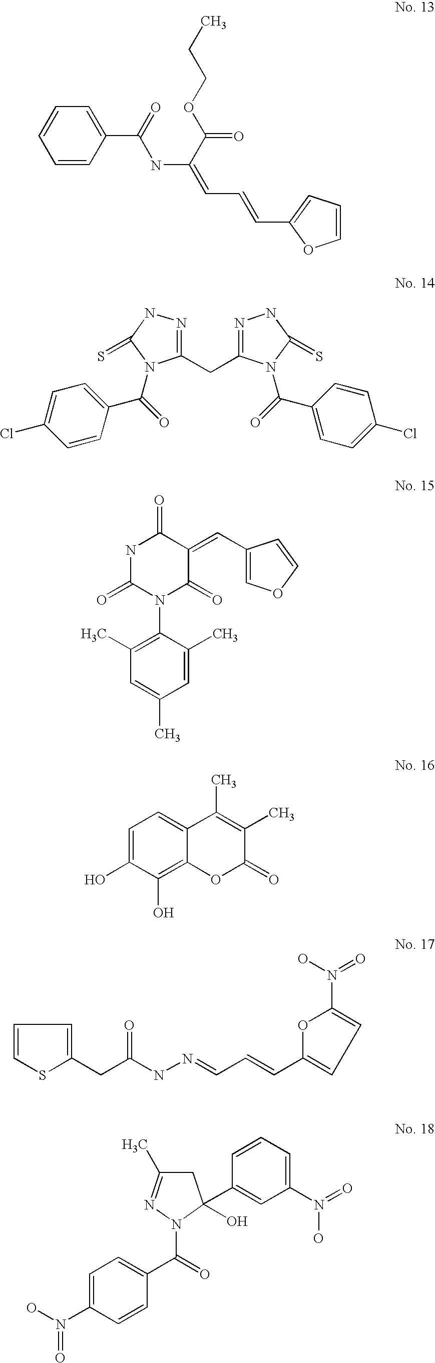

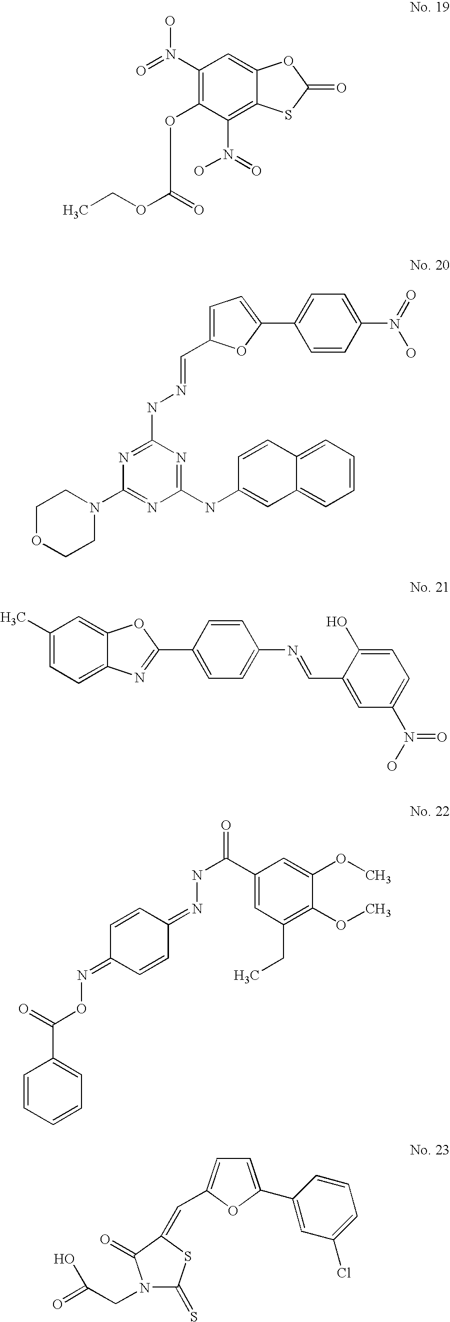

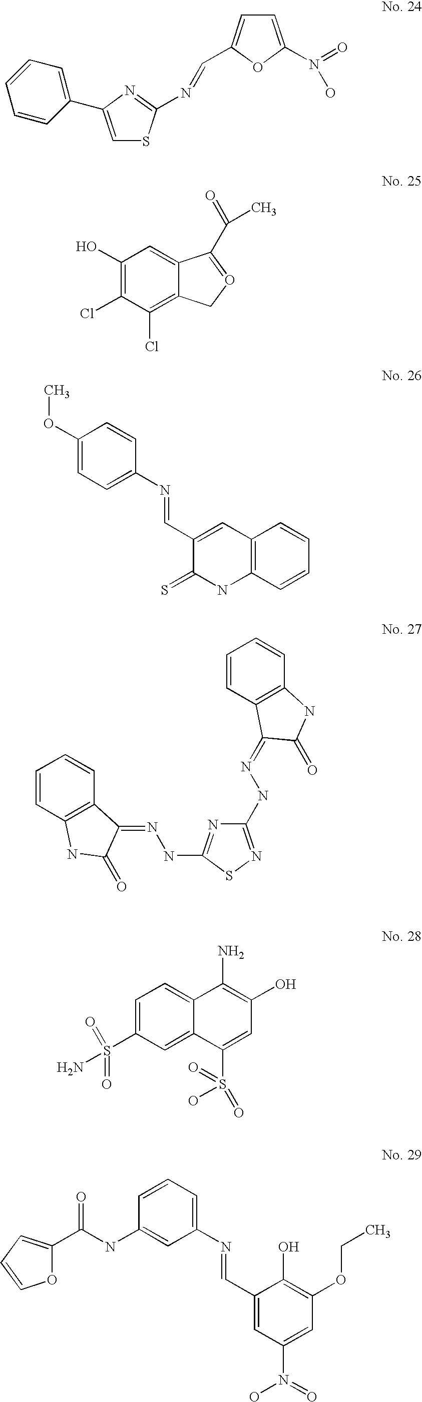

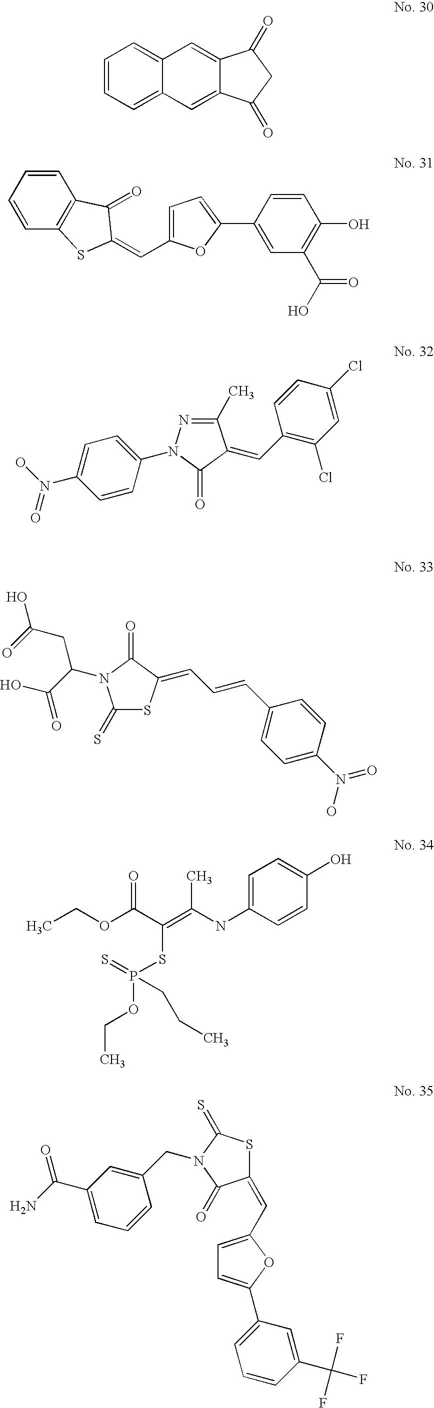

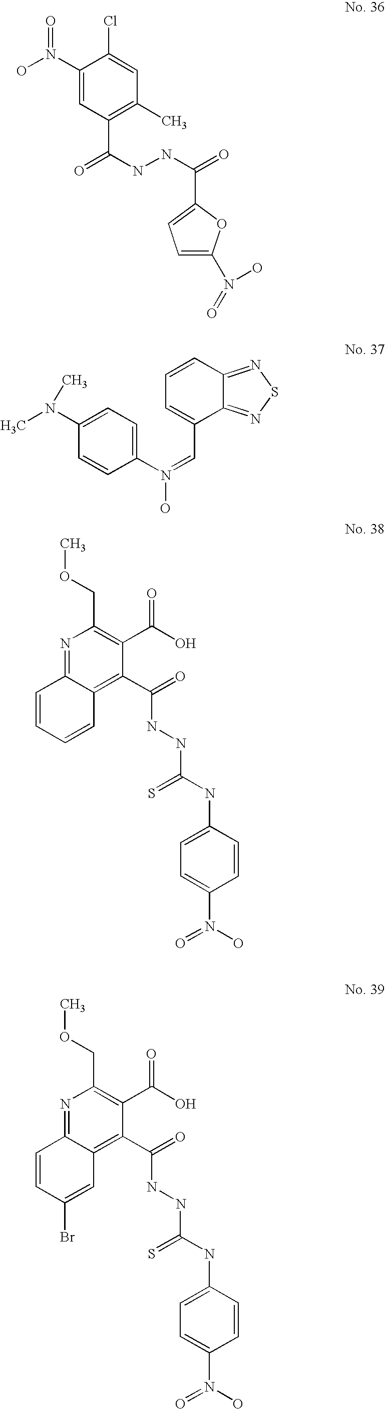

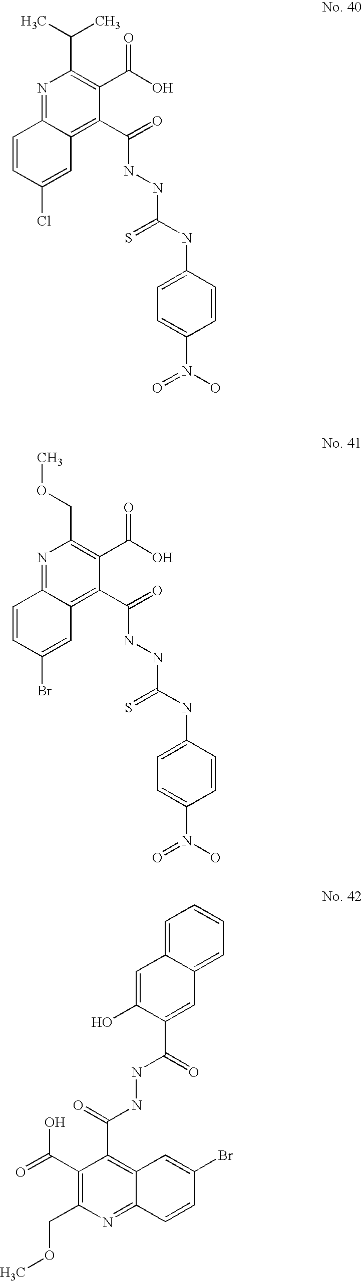

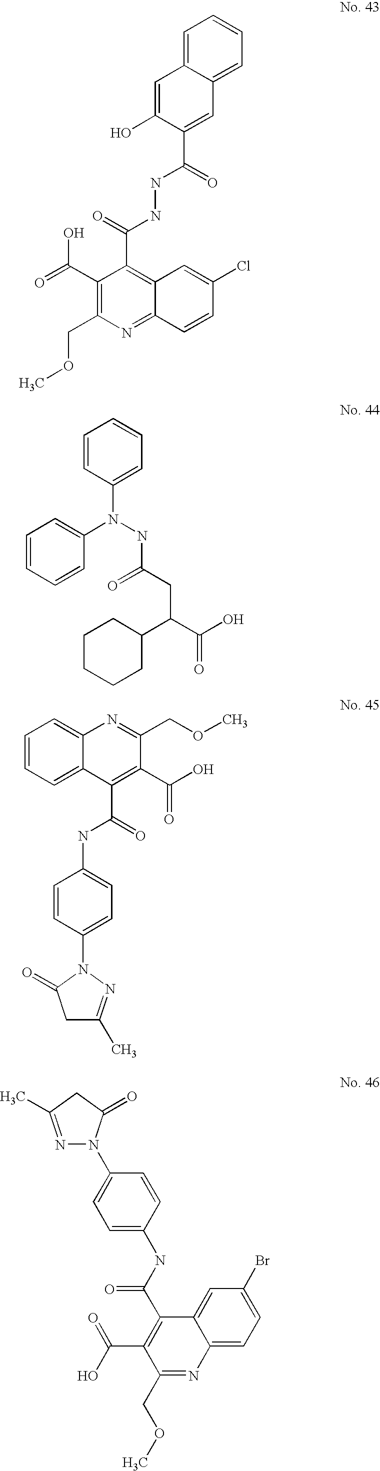

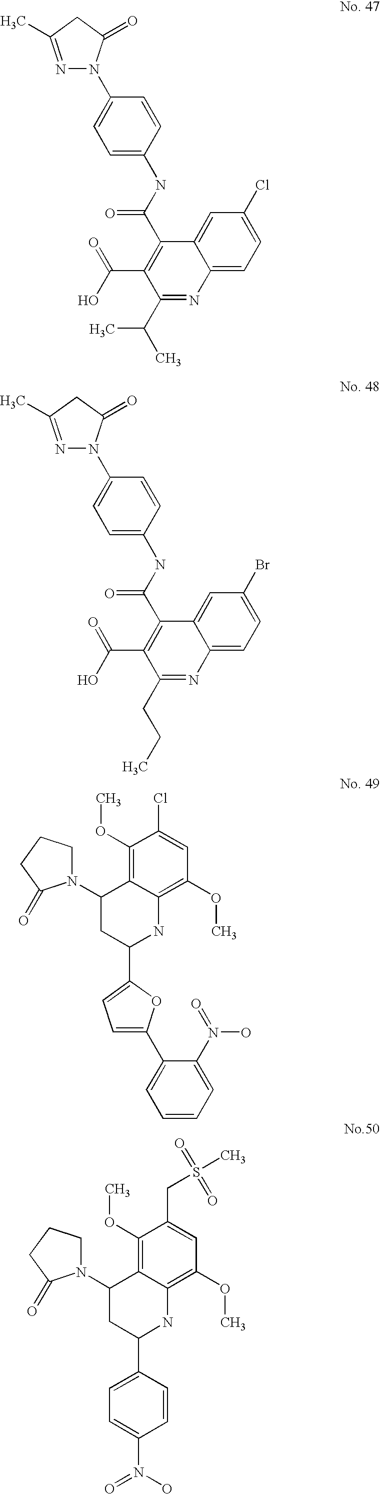

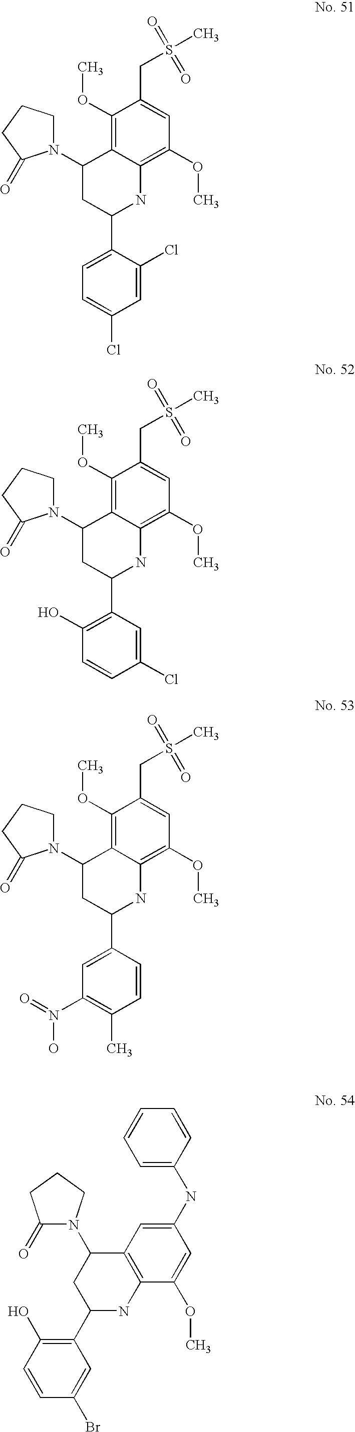

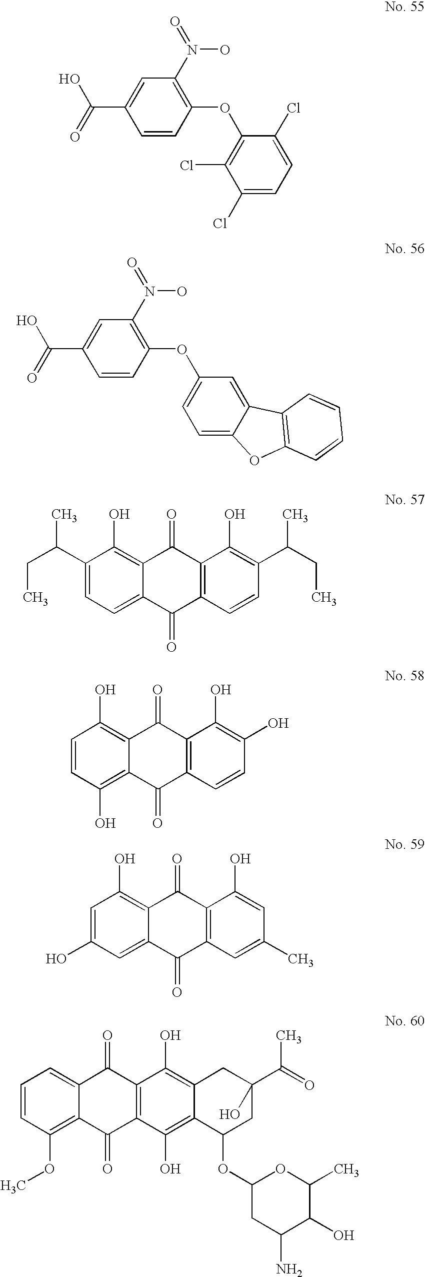

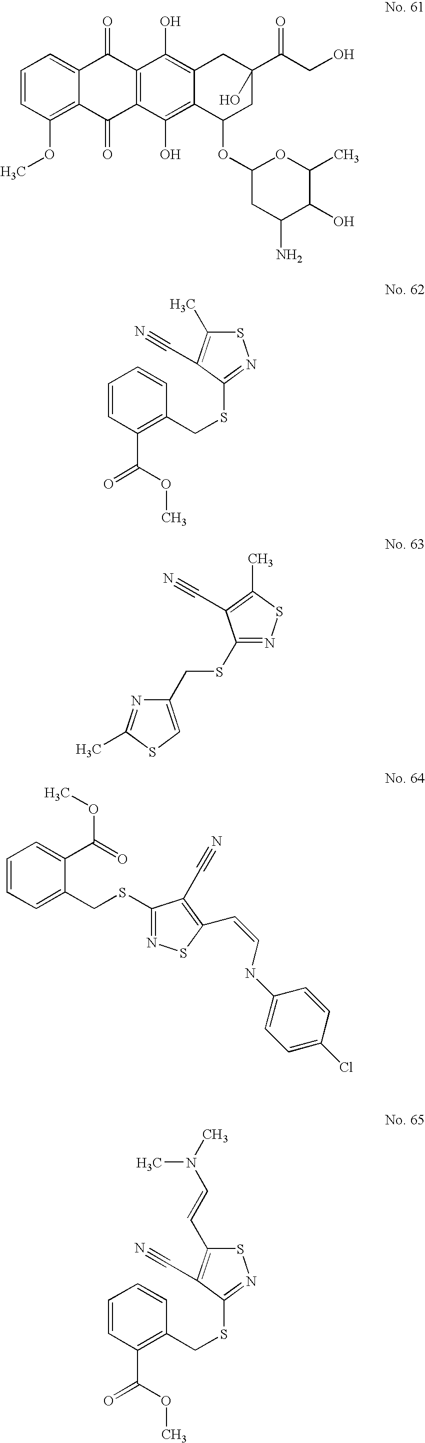

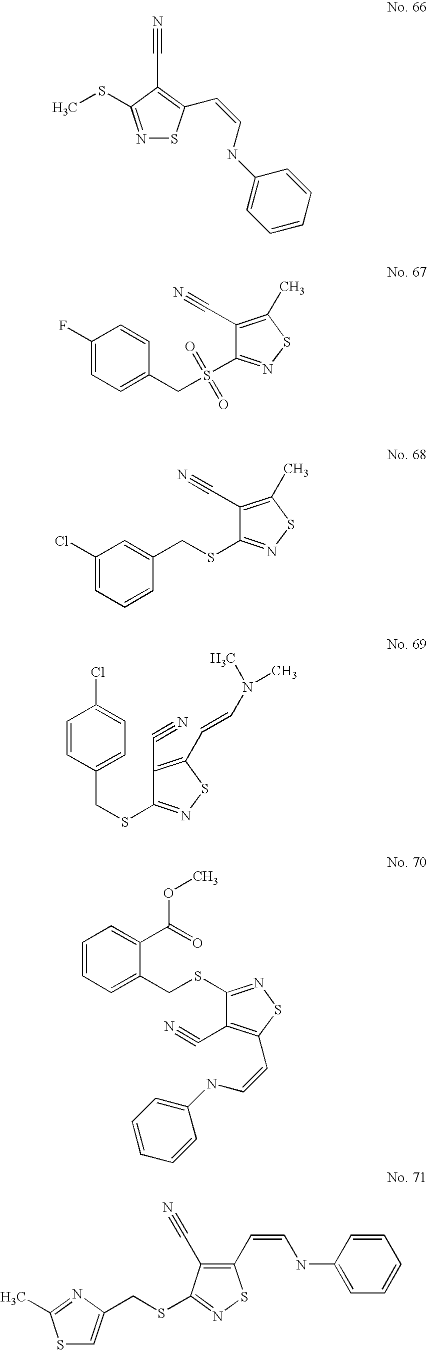

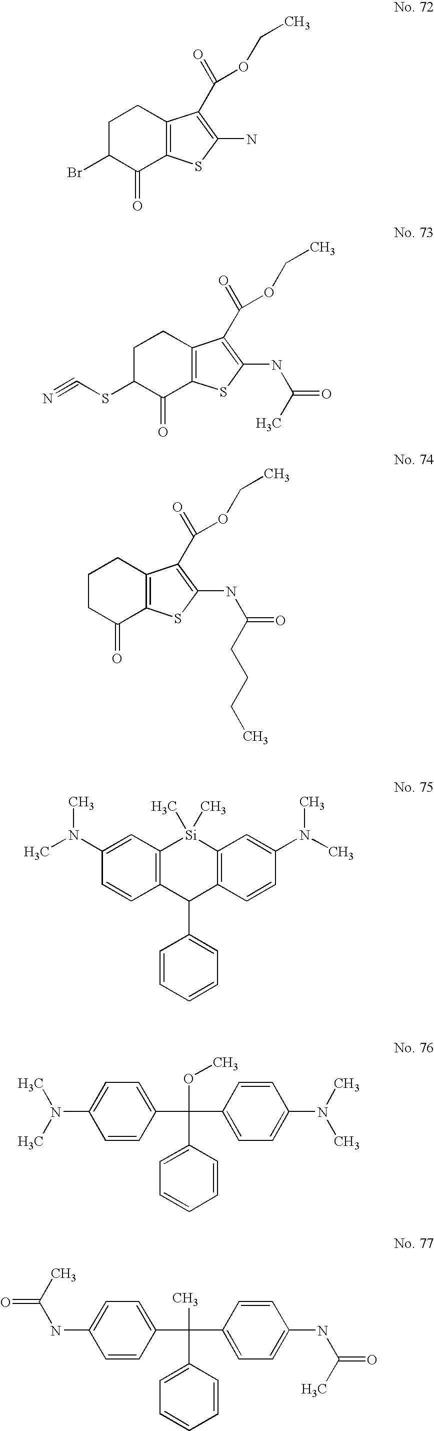

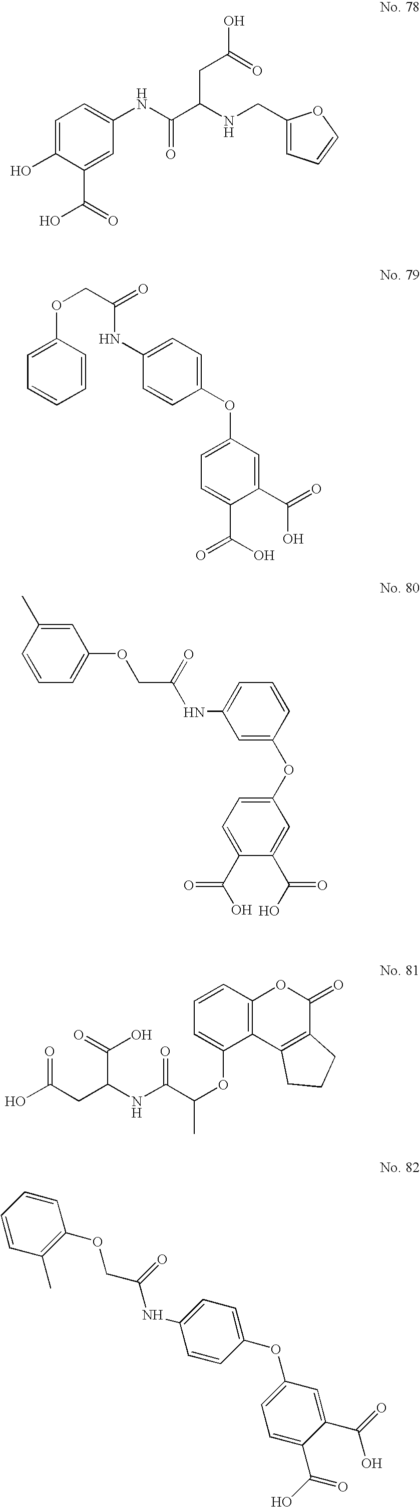

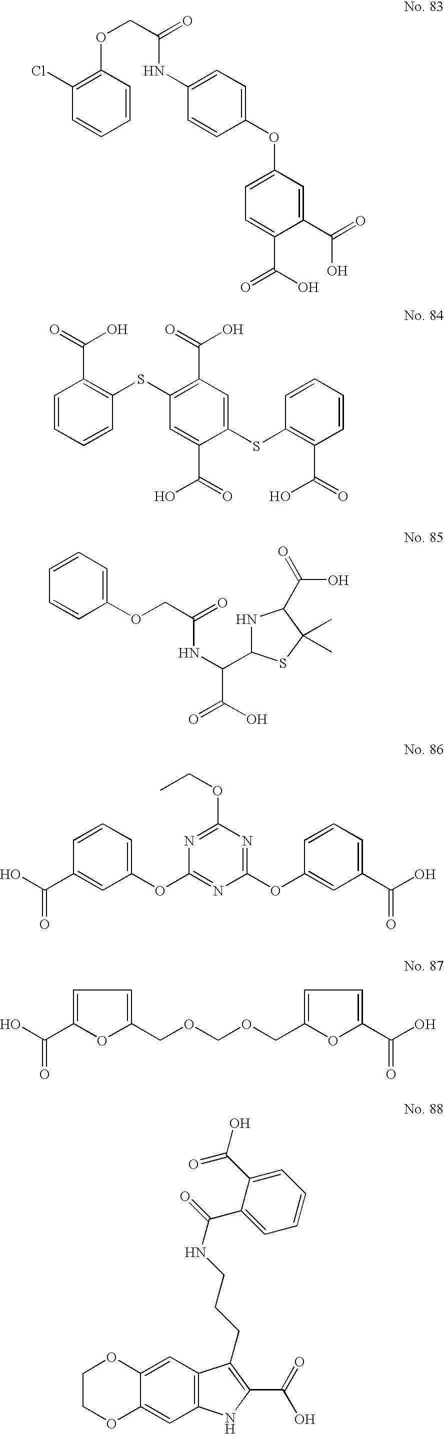

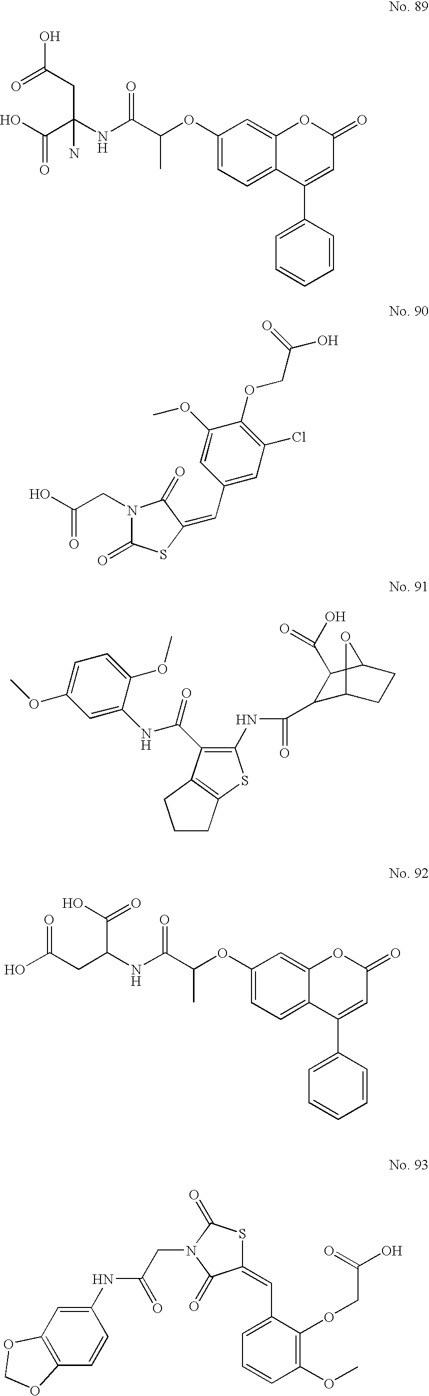

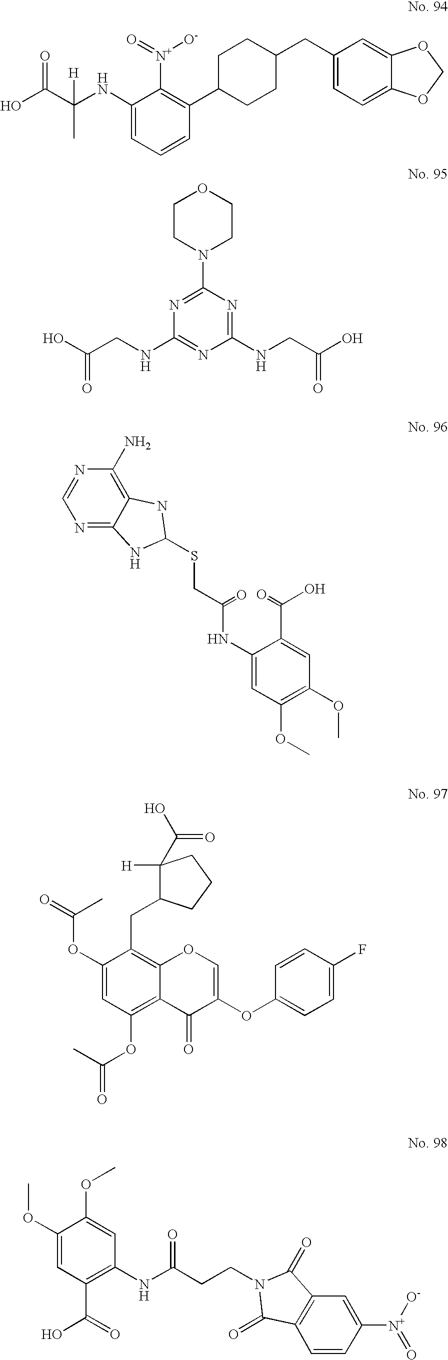

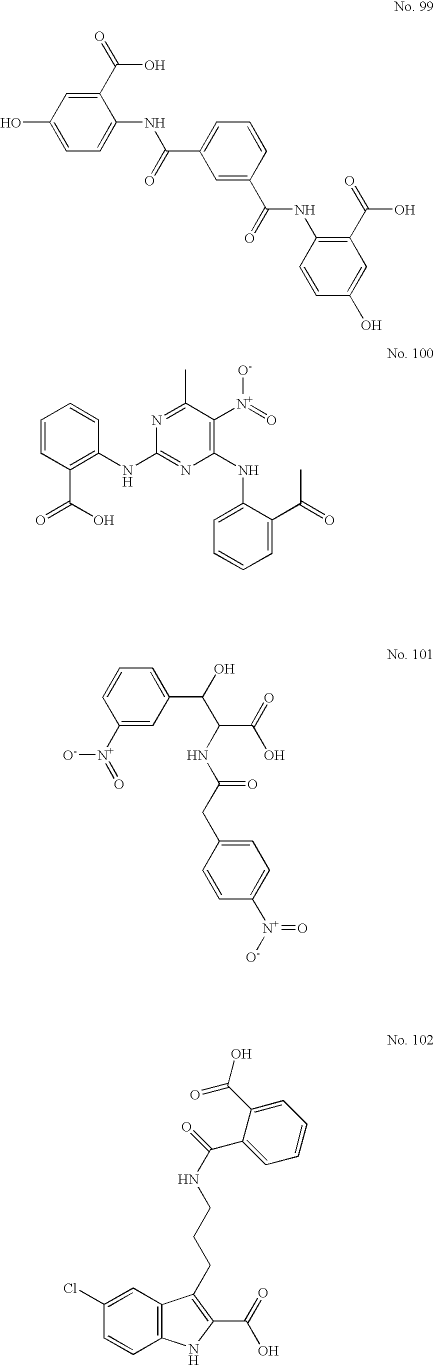

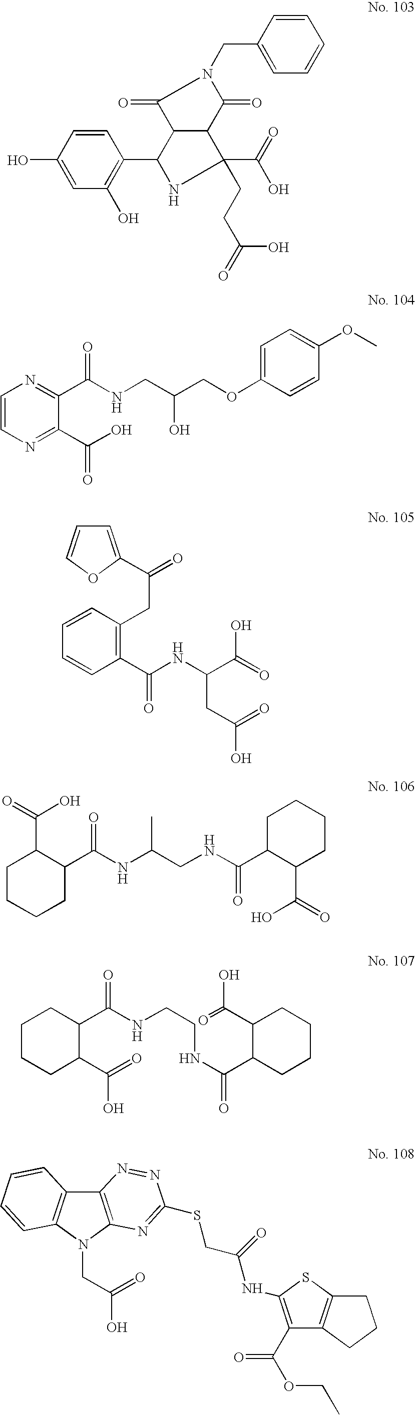

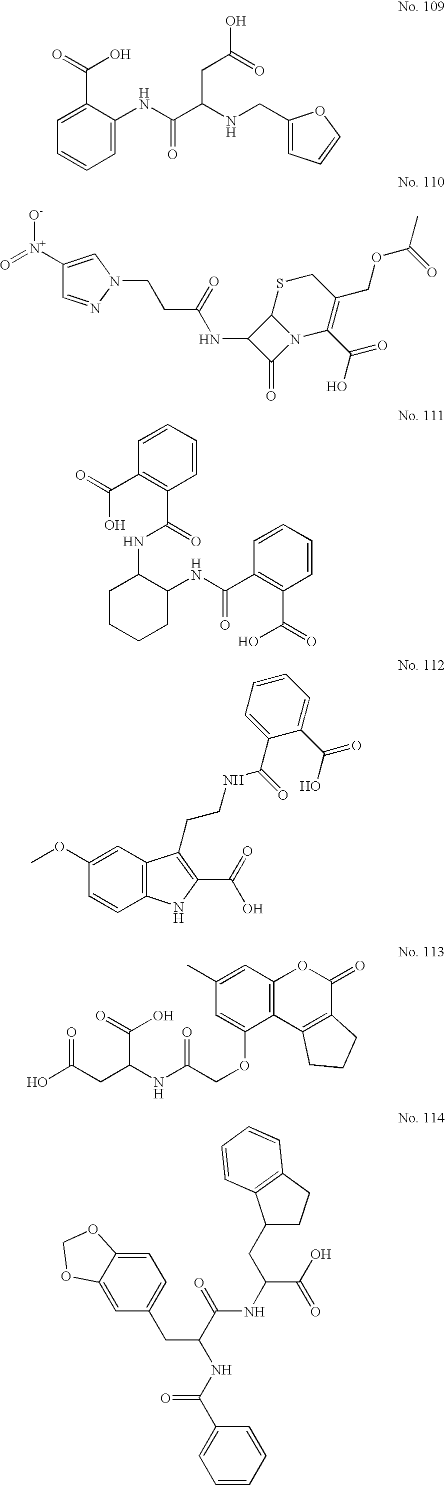

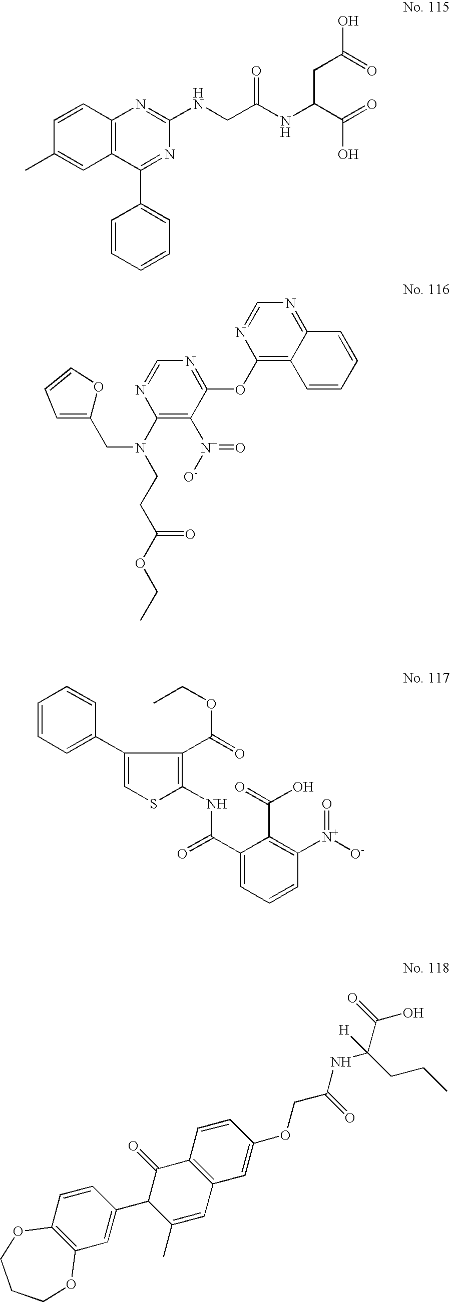

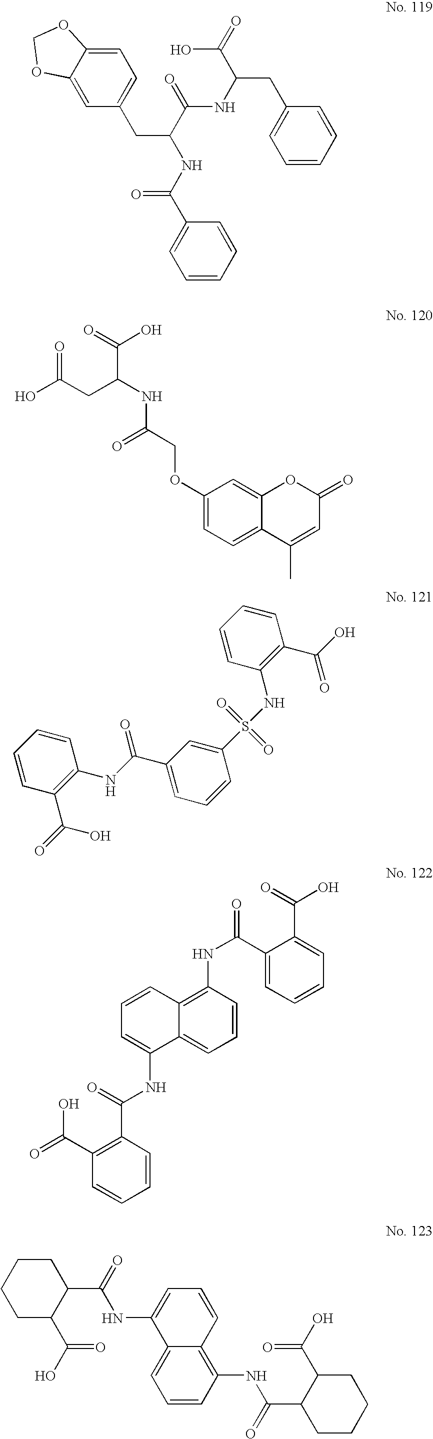

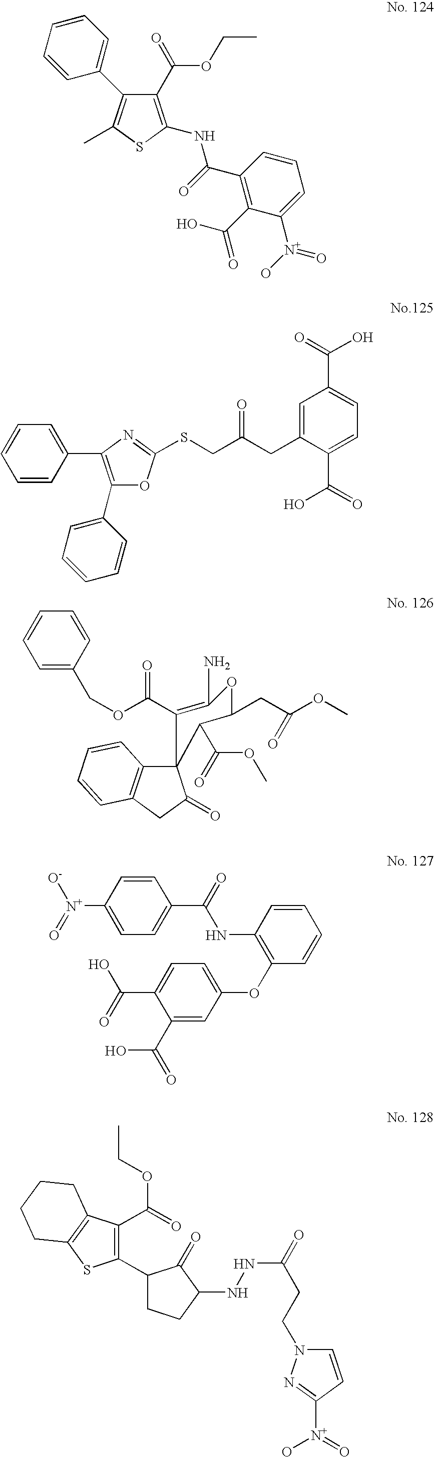

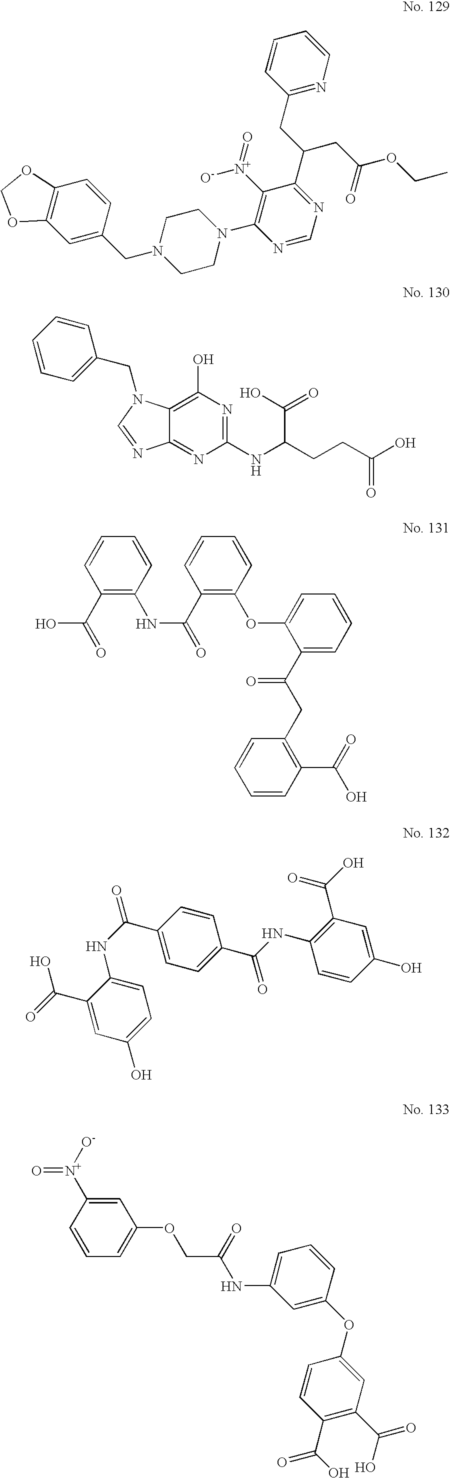

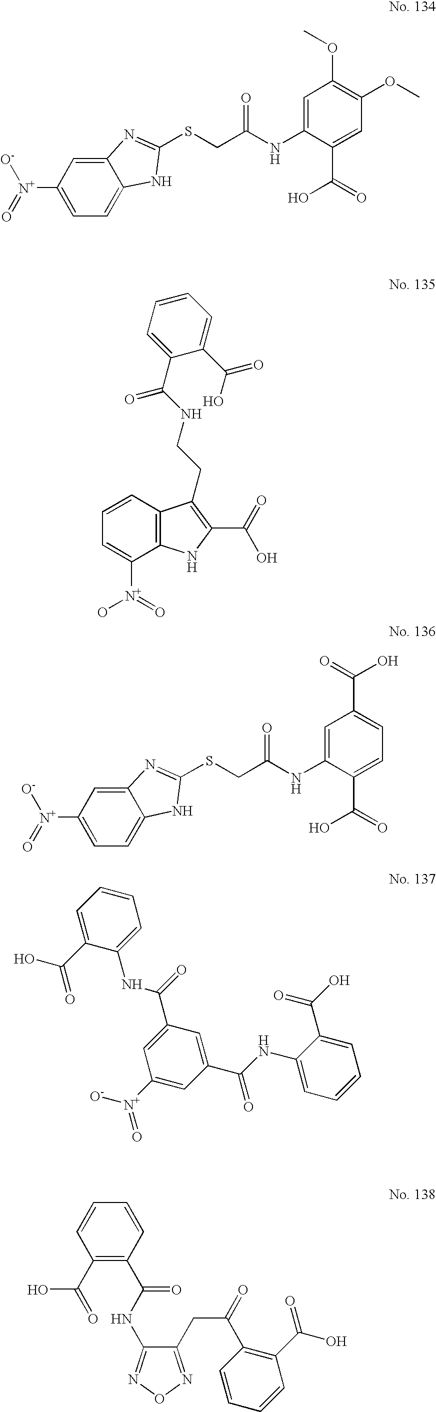

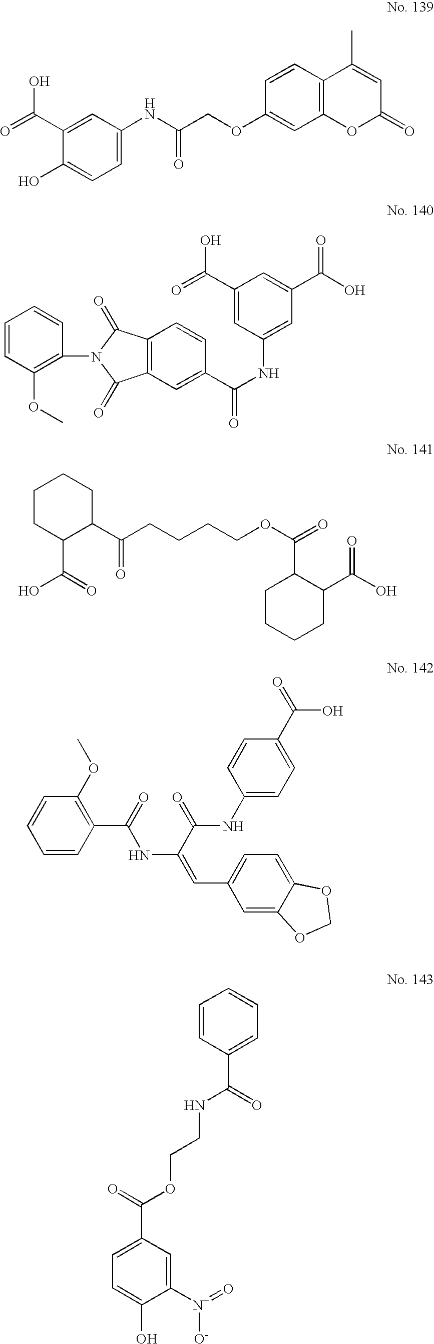

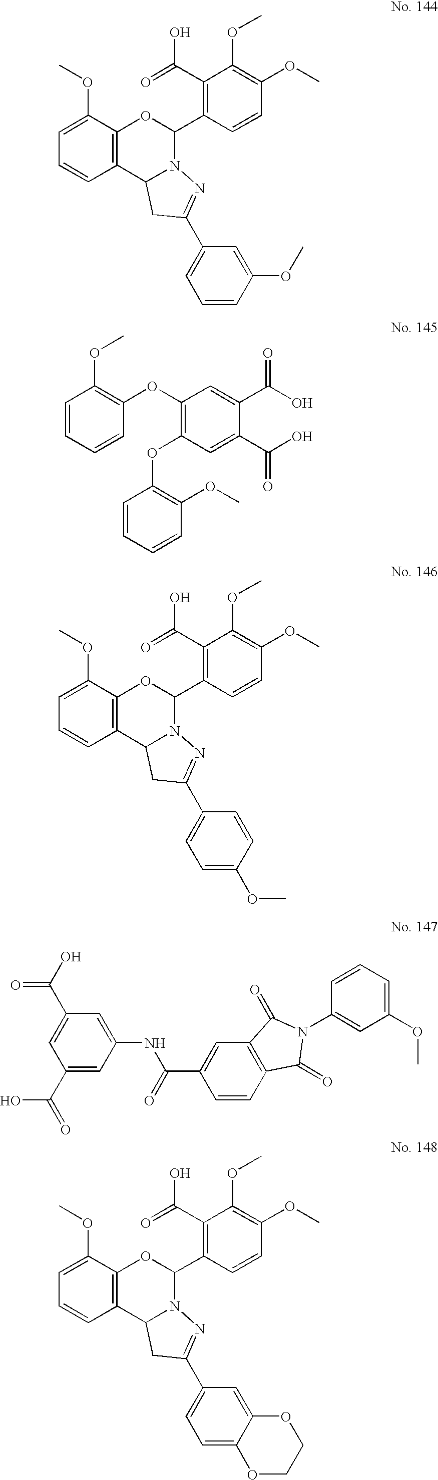

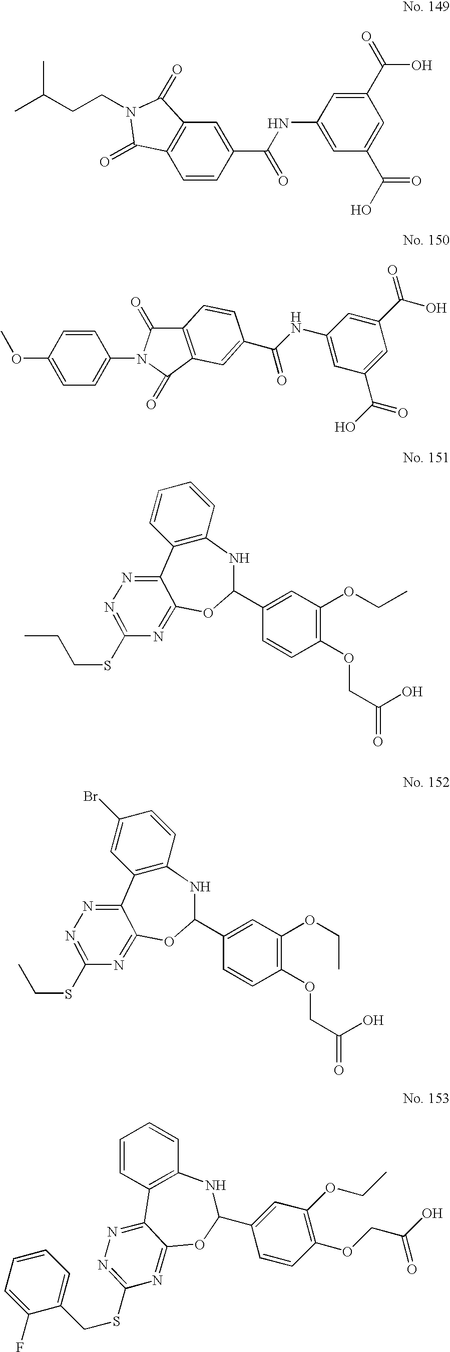

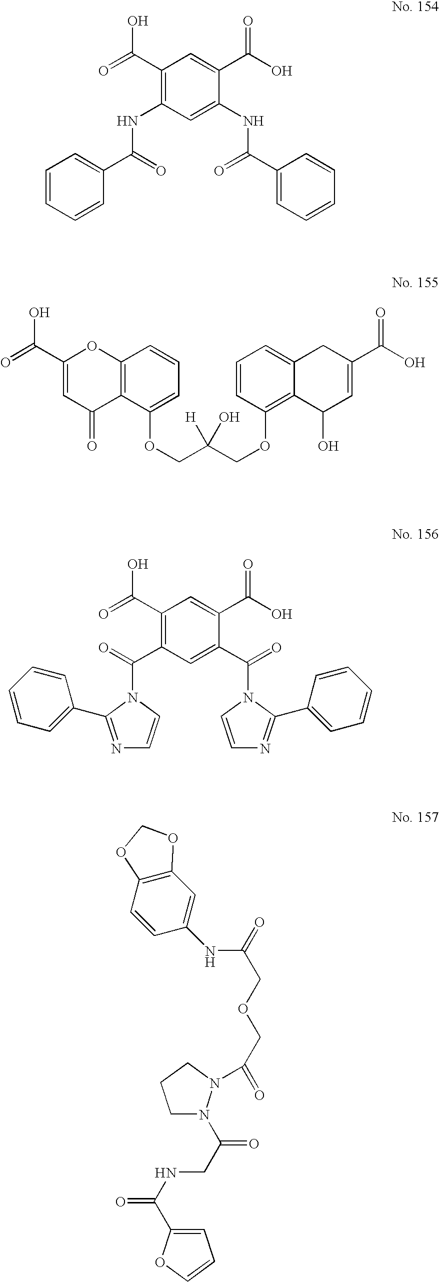

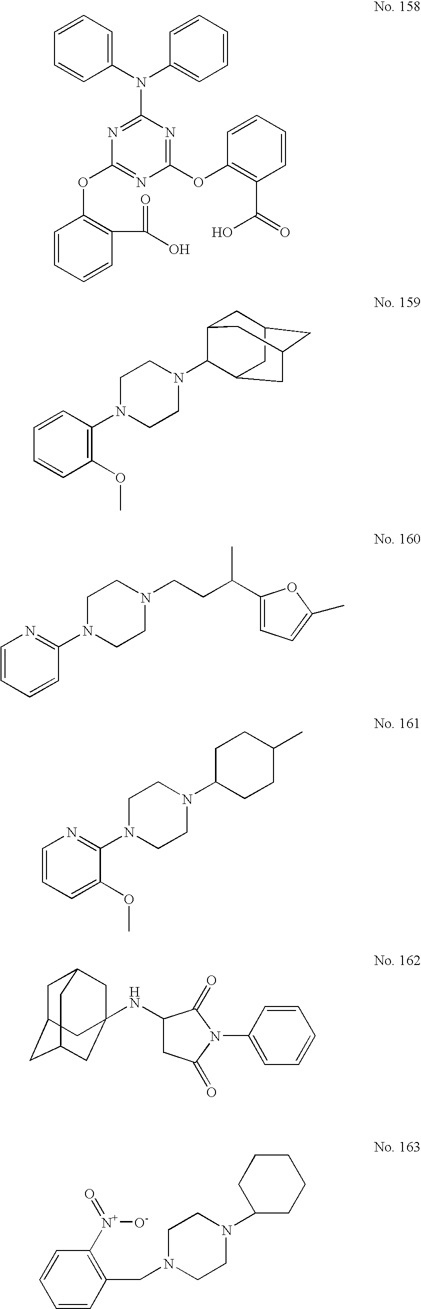

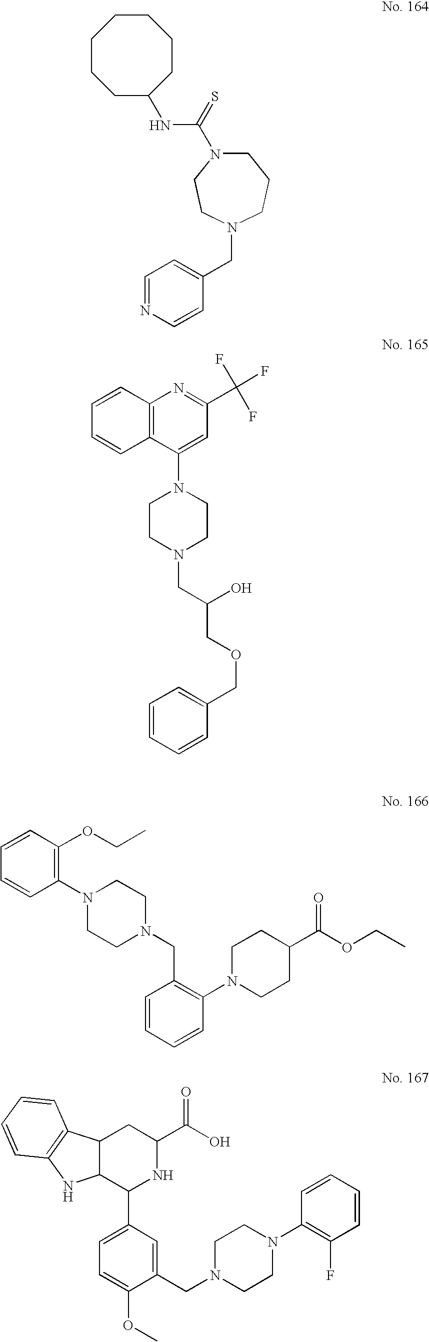

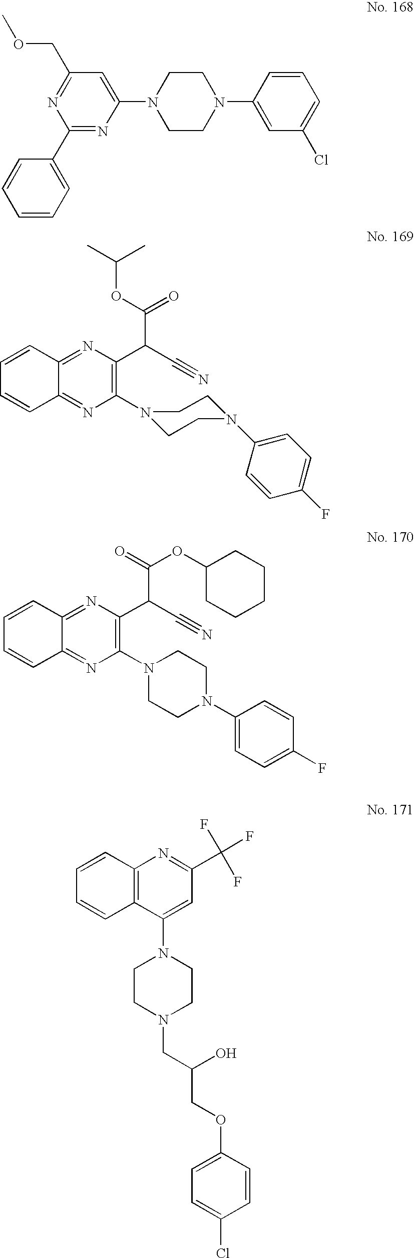

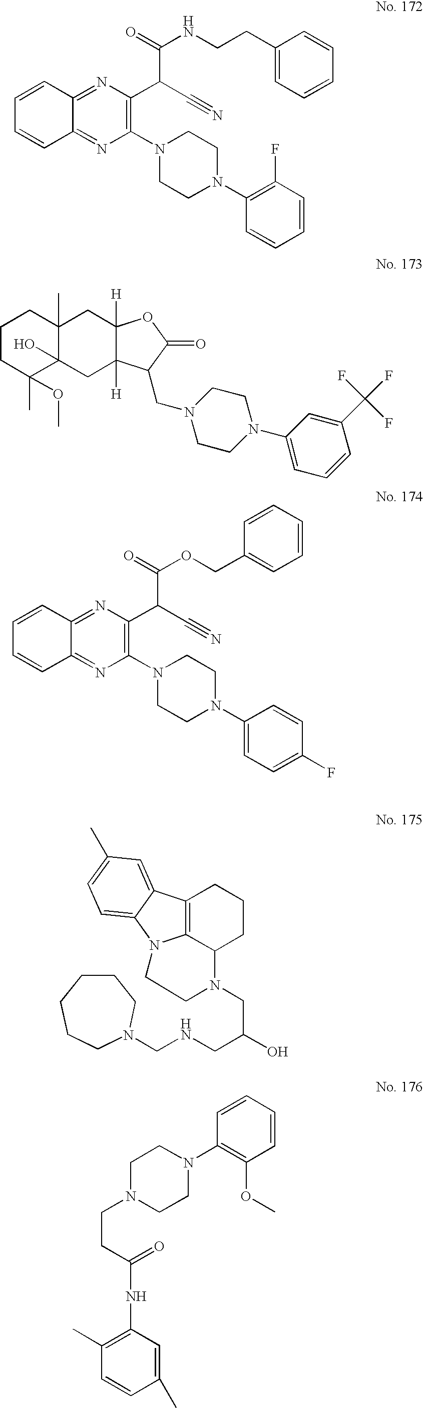

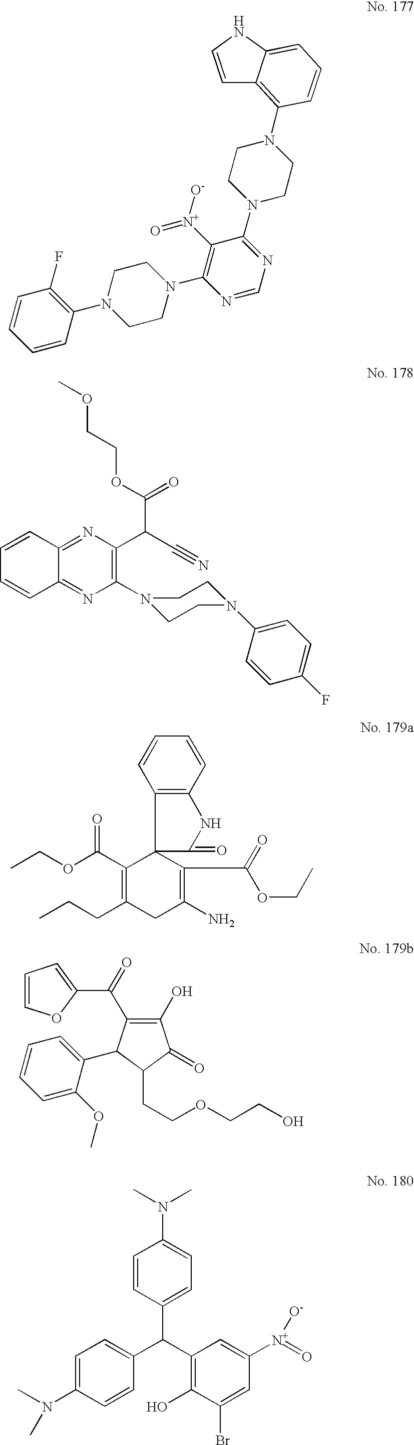

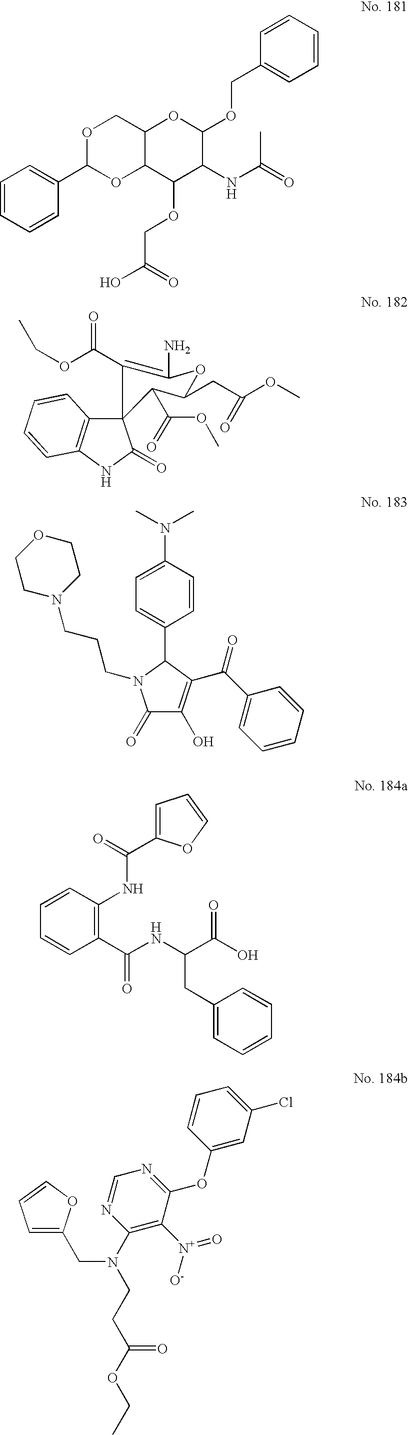

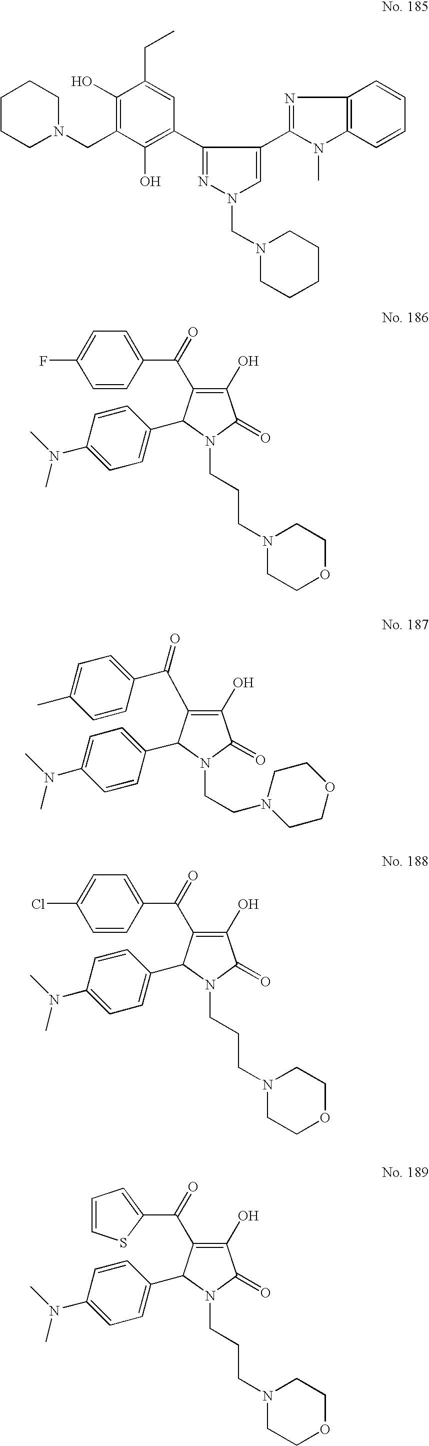

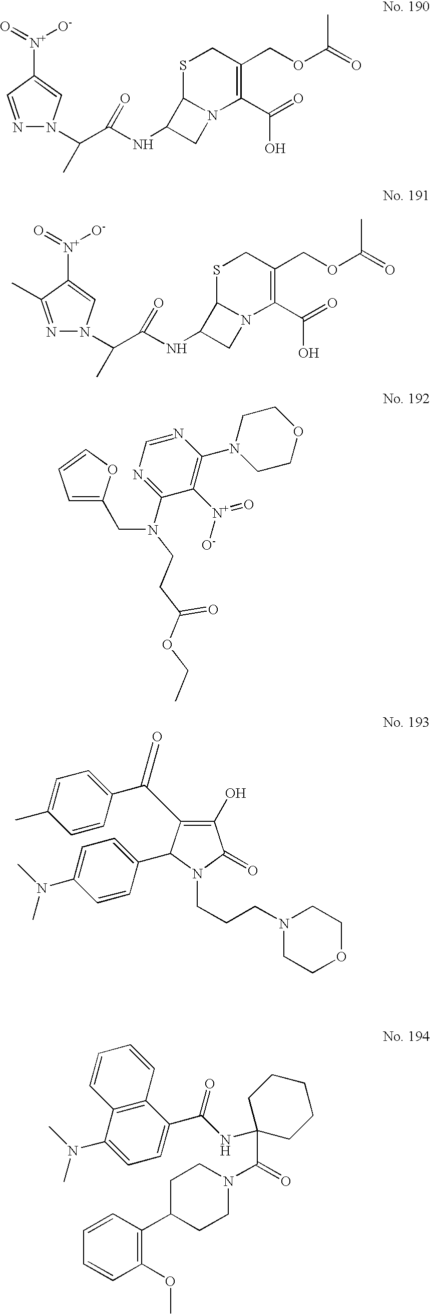

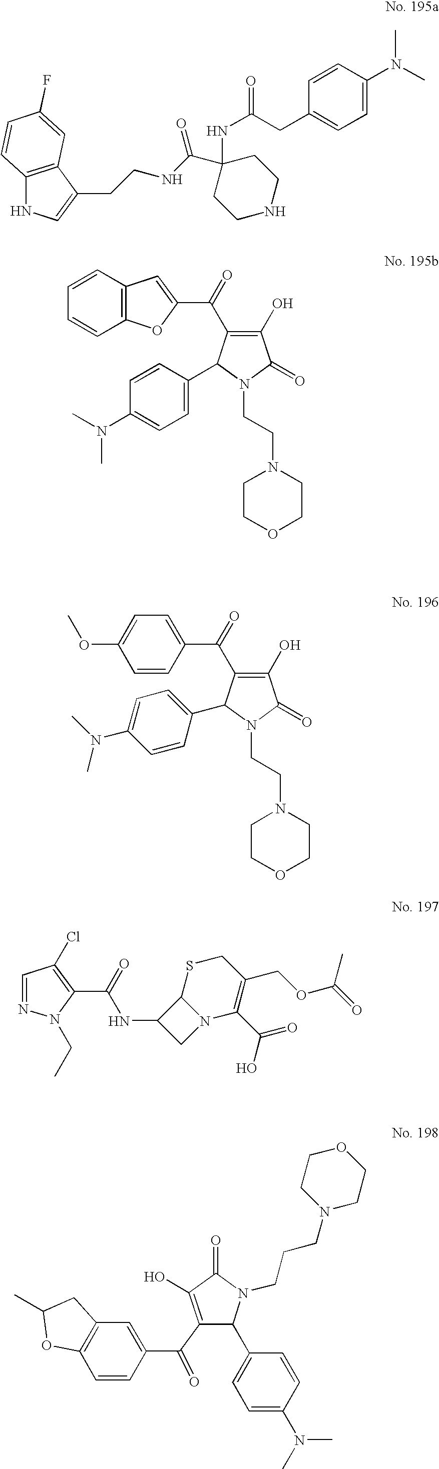

The following compounds were obtained from Maybridge plc, Trevillett, Tintagel, Cornwall PL34 OHW, England: Nos.: 1 and 7.

The following compounds were obtained from Interchim, 213 av J F Kennedy, BP1140, 03103 Montlucon Cedex, France: Nos.: 2, 6, 8, 10, 11, 18, 19, 21, 25, 26, 60 and 62-71.

The following compounds were obtained from ASINEX Ltd., Moscow, Russia:

Nos.: 9 and 29.

The following compounds were obtained from Ambinter SARL, 46 quai Louis Blériot, F-75016 Paris, France:

Nos.: 13, 23, 24, 33 and 34.

The following compounds were obtained from ChemBridge Corporation, San Diego, Calif. 92127, USA:

Nos.: 15 and 58.

The remaining compounds were obtained from Merck KgaA, Frankfurter Str. 250, 64293 Darmstadt, Germany.

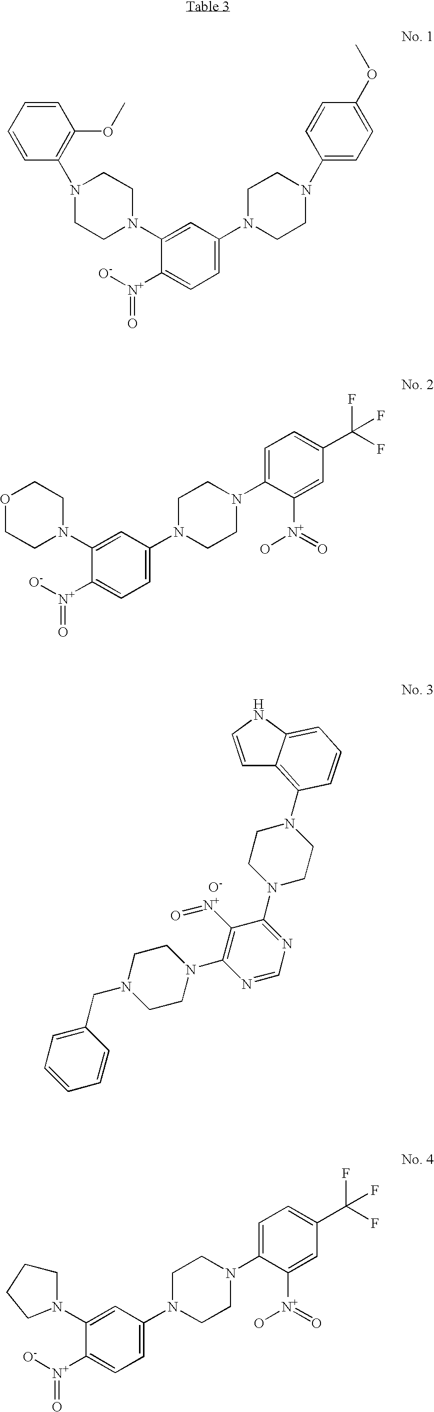

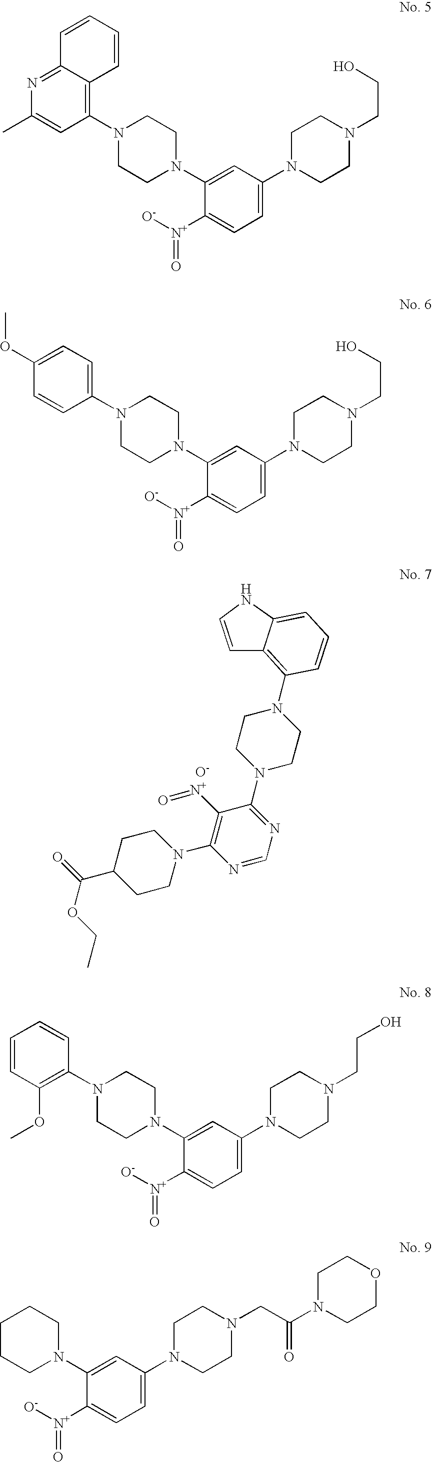

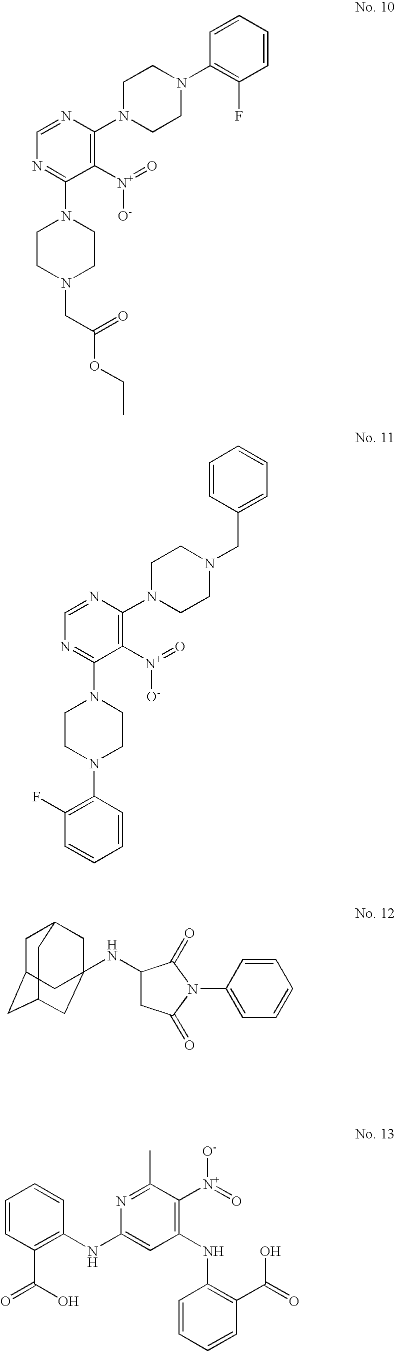

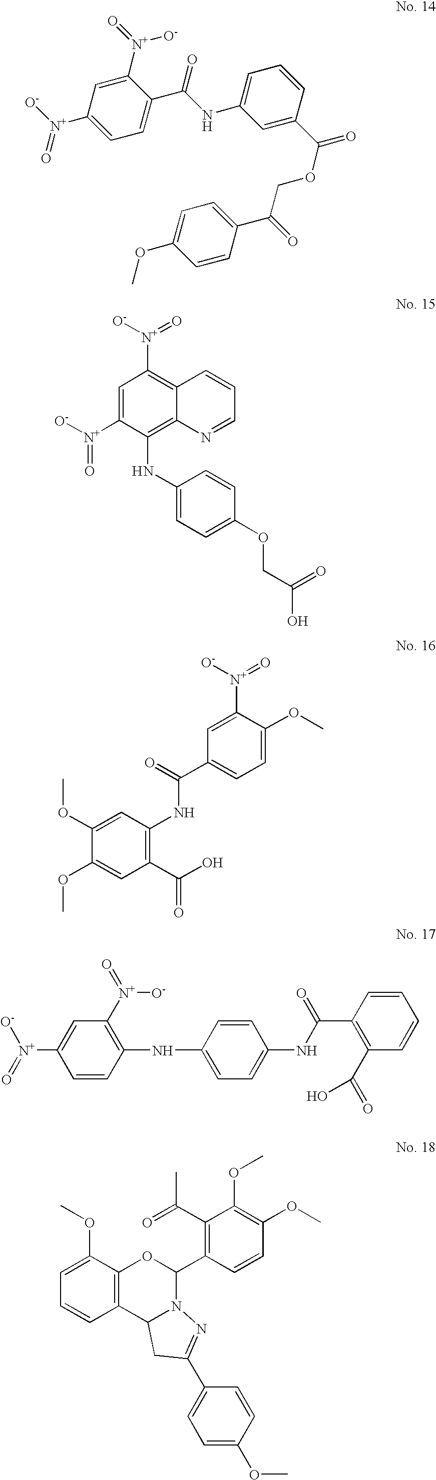

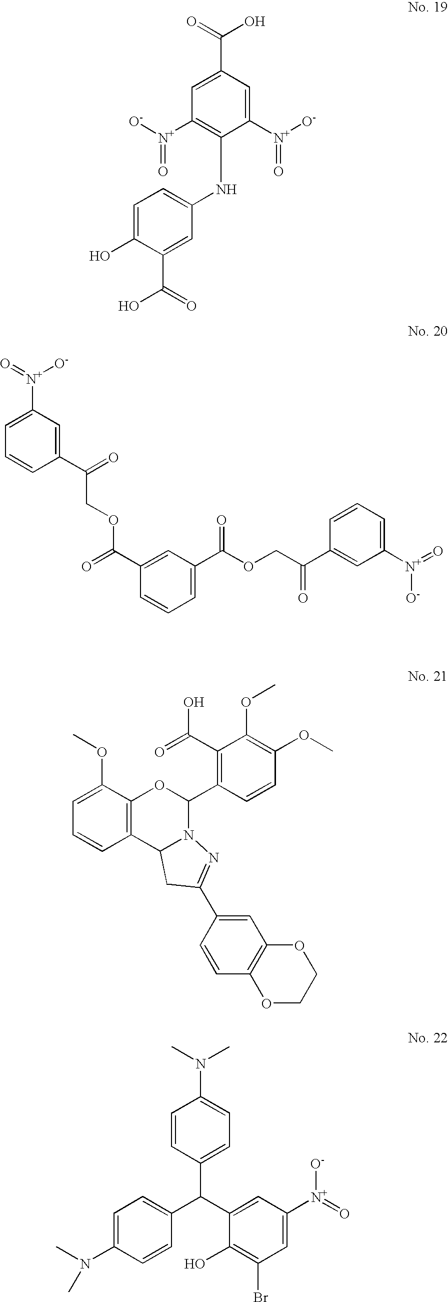

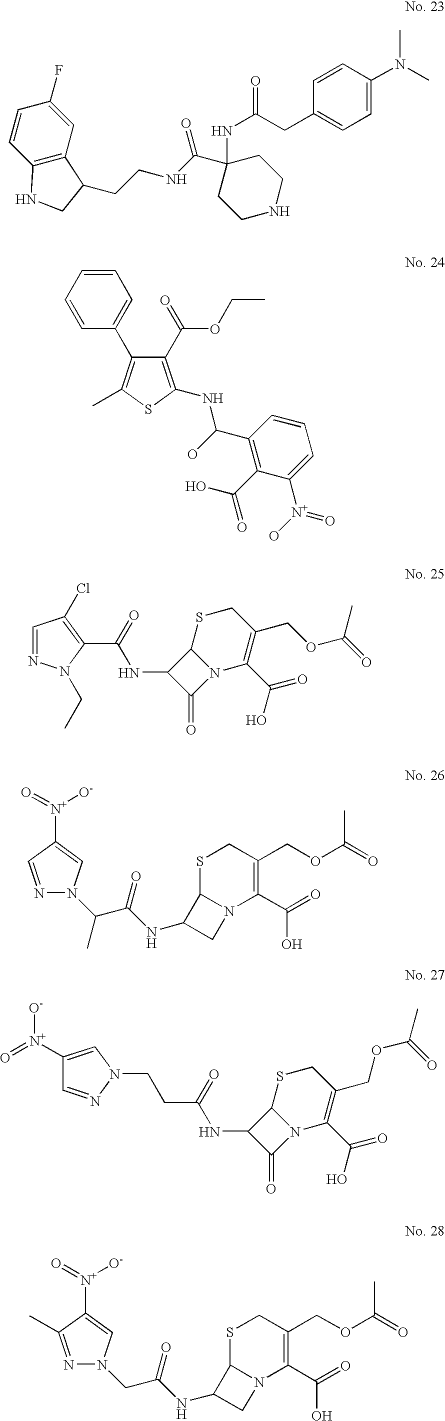

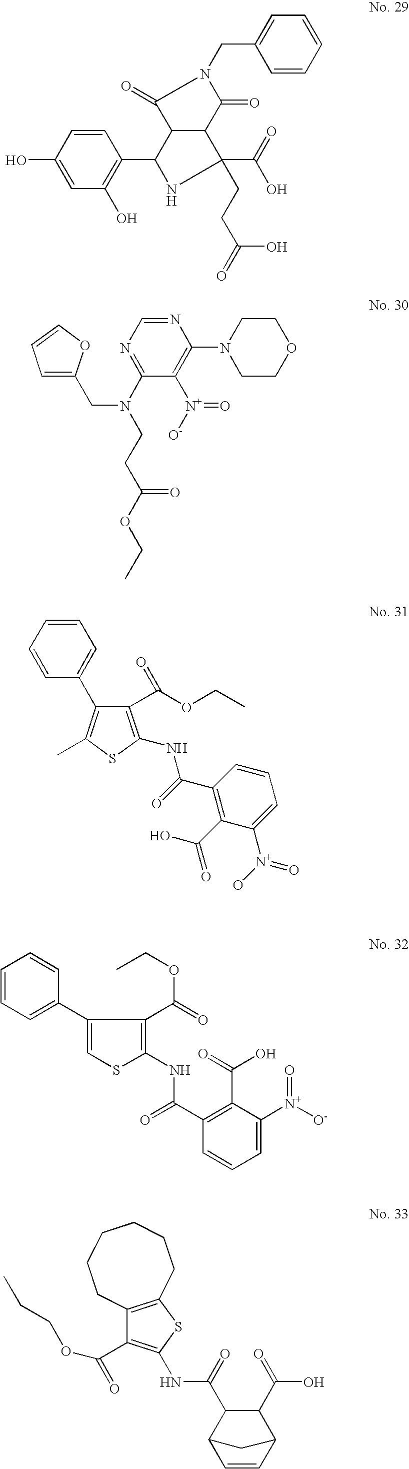

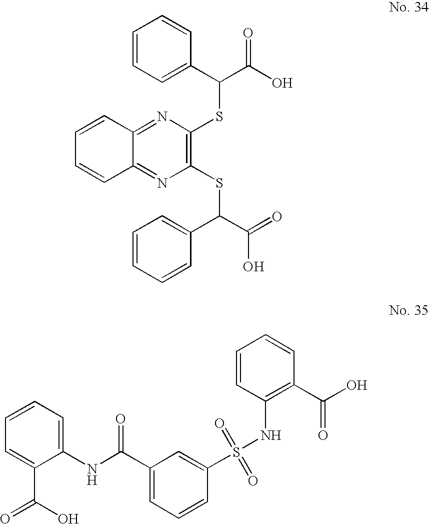

The following compounds were obtained from TimTec Corporation, 100 Interchange Blvd., Newark, Del. 19711, USA:

Nos.: 1, 25, 26, 27, 28 and 29.

The following compounds were obtained from Tripos Inc. Louis, Mo., 63144, USA:

Nos.: 3, 7, 10, 11 and 30.

The following compounds were obtained from ChemBridge Corporation, San Diego, Calif. 92127, USA:

Nos.: 5, 6, 8, 17, 31, 32, 34 and 35.

The following compounds were obtained from SPECS Corporation, 2628 XH Delft, Netherlands:

Nos.: 2, 4, 9, 14, 19 and 33.

The following compounds were obtained from Vitas-M Laboratory Ltd., Center of Molecular Medicine Vorob'evi Gori, Moscow, Russia:

Nos.: 15 and 16.

The following compounds were obtained from ASINEX Ltd., Moscow, Russia:

Nos.: 13 and 20.

The following compounds were obtained from InterBioScreen Ltd., 121019 Moscow, Russia:

Nos.: 18 and 21.

The following compounds were obtained from Merck KgaA, Frankfurter Str. 250, 64293 Darmstadt, Germany: Nos.: 12, 22, 23 and 24.

1. A method for treating a neurodegenerative condition comprising administering a pharmaceutical composition comprising a therapeutically effective amount of a compound that inhibits protein aggregate formulation and depolymerizes protein aggregates.

2. The method of claim 1 wherein the compound has the general formula LSA 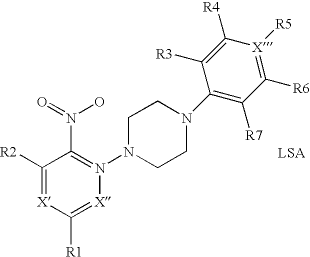
wherein R1 and R2 are selected from H and 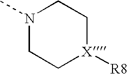
R3 is selected from H, OCH₃, and F;
R4 is selected from H and CH₃, or R2 and R4 are connected to form a condensed pyrrole ring;
R5, if present, is selected from H and OCH₃;
R6 is H and R7 is H, or R6 and R7 are connected to form a condensed phenyl ring;
R8 is selected from CH₂CH₂OH, CH₂Ph and C(O)OCH₂CH₃, and;
X′, X″, X′″, and X″″ are selected from N and C.

3. The method of claim 2 wherein the compound is selected from the group consisting of

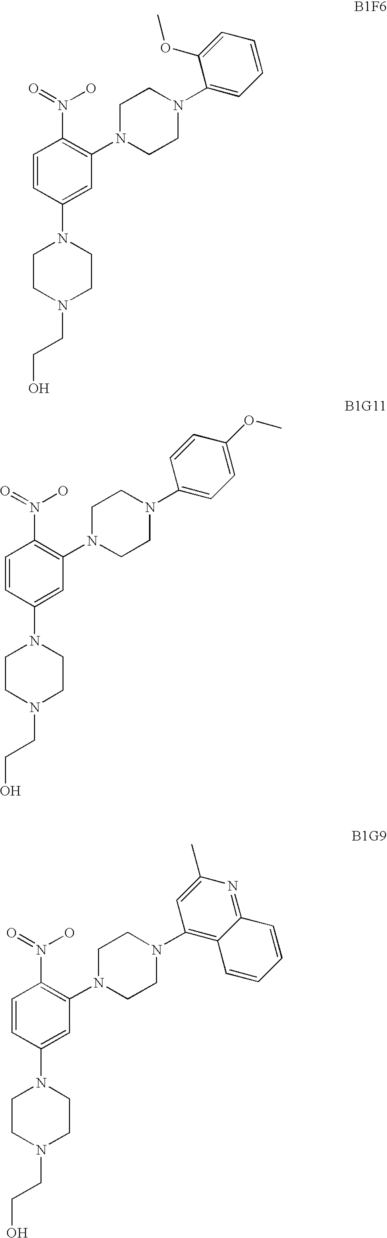

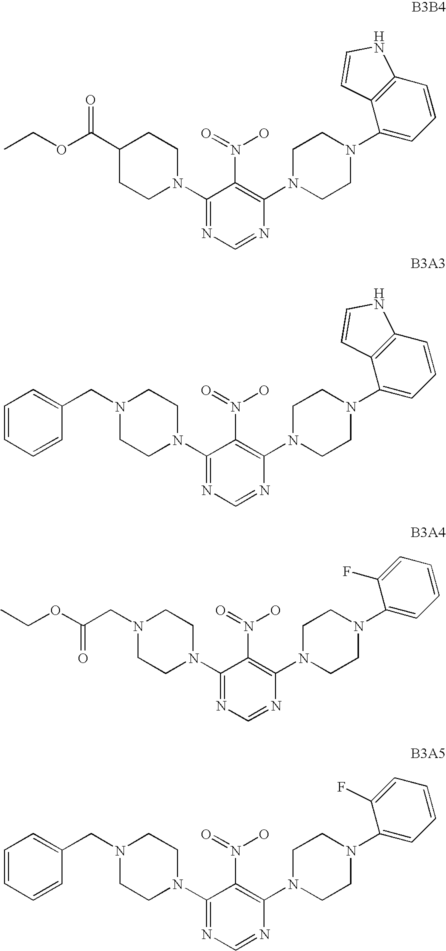

4. The method of claim 1 wherein the compound has the formula LSB 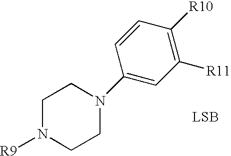
wherein R9 is selected from 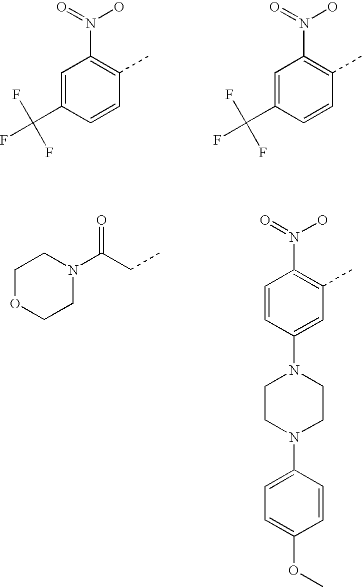
R10 is selected from H and NO₂, and
R11 is selected from an N-morpholino group, N-pyrrolidino group and OCH₃.

5. The method of claim 4 wherein the compound with the general formula LSB is selected from

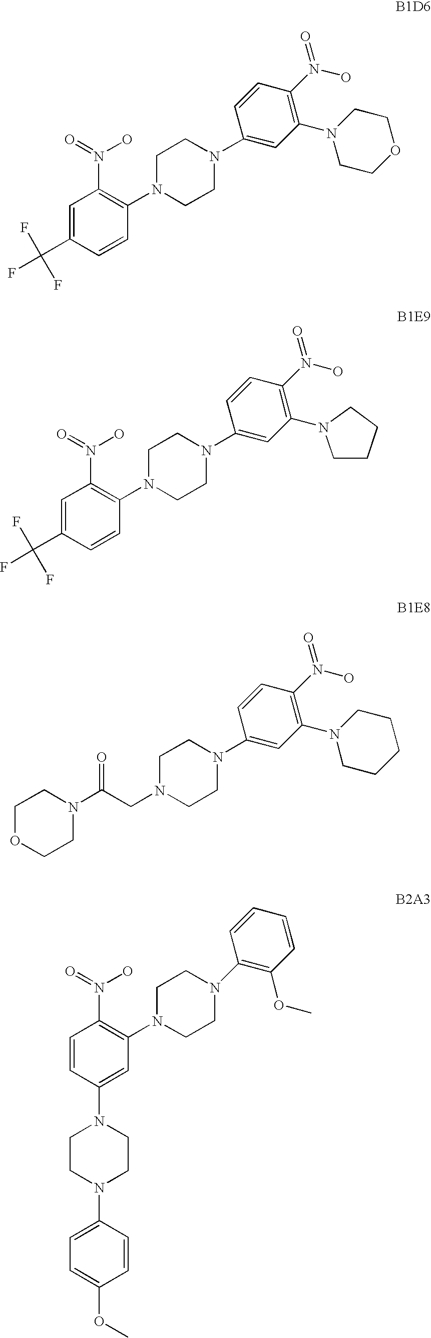

6. The method of claim 1 wherein the compound is selected from

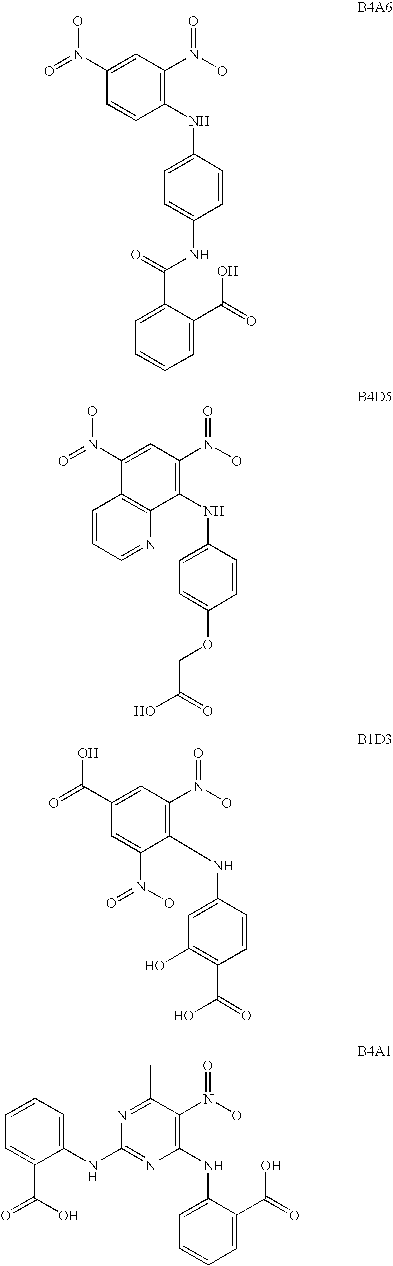

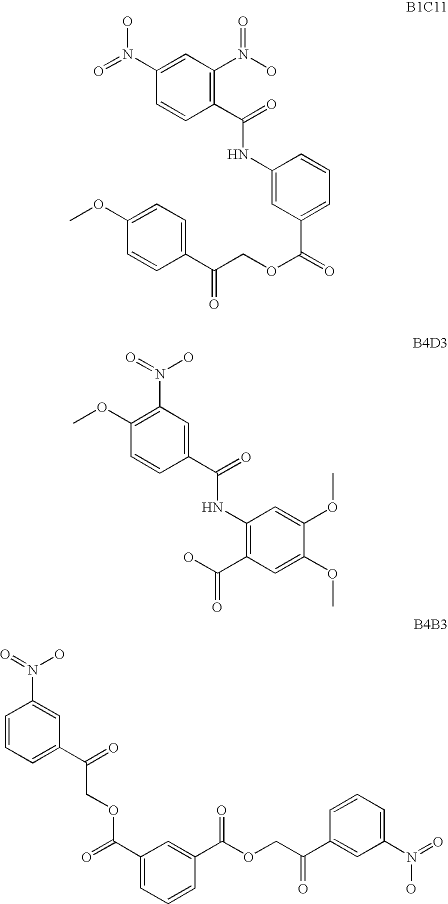

7. The method of claim 1, wherein the protein aggregate comprises PHFs consisting of tau protein.

8. The method of claim 1, wherein the protein aggregate comprises Aβ protein, prion protein, or α-synuclein.

9. The method of claim 1, wherein the neurodegenerative condition is Alzheimer disease.

10. The method of claim 1, wherein the neurodegenerative condition is selected from the group of Tauopathies consisting of CBD (Cortical Basal Disease), PSP (Progressive Supra Nuclear Palsy), Parkinsonism, FTDP-17 (Fronto-Temporal Dementia with parkinsonism linked to chromosome 17), Familiar British Dementia, Prion Disease (Creutzfeld Jakob Disease) and Pick's Disease.

11. The method of claim 1, wherein the pharmaceutical composition is administered orally or parenterally.

12. The method of claim 1, wherein the pharmaceutical composition is administered as part of a sustained release formulation or administered by depot implantation.

13. The method of claim 1, wherein the compound selected from the group consisting of compounds 1 to 374 as shown in Table 1 and 1 to 34 as shown in Table 3.

14. The method of claim 13, wherein the compound selected from the group consisting of compounds 1 to 34 as shown in Table 3 and the protein aggregates comprise PHFs consisting of tau protein.

15. A genetically modified cell line, wherein tau gene expression can be induced.

16. The genetically modified cell line of claim 15, wherein the cell line is modified to express a mutant of tau that polymerizes in neurons into aggregates, which aggregates can be visualized by thioflavine S.

17. The cell line of claim 15, wherein the cell line is a N2a cell line.

18. The cell line of claim 15, wherein a tet-on system is used for regulation of expression.

19. The cell line of claim 16, wherein the mutant of tau is a construct comprising four microtubule binding repeats of tau and comprising (a) deletion of lysine at position 280 (K280) or (b) mutations of isoleucines 277 and 308 into prolines (I277P and I308P).

20. The cell line of claim 19, wherein the mutant of tau is a mutant of K18 bearing a deletion at K280 and isoleucines 277 and 308 are mutated into prolines (I277P and I308P).

21. A method for identifying an agent to attenuate or to inhibit aggregation of tau comprising the step of comparing tau aggregation in the cell line of claim 15 in the presence and absence of the agent, wherein an increase in aggregation identifies the agent as an attenuator, and a decrease in aggregation identifies the agent as an inhibitor.

22. The method of claim 21, wherein the tau is a mutant of tau.

23. The method of claim 21, wherein the agent is a compound selected from the group consisting of compounds 1 to 374 as shown in Table 1, a proteins an antibody, a fatty acid, a nucleotide, or a ribonucleic acid.

24. (canceled)

25. (canceled)

26. A transgenic non-human animal which expresses a mutant of tau that polymerize in neurons into aggregates.

27. The transgenic non-human animal of claim 26, wherein the animal is a transgenic mouse.

28. The transgenic non-human animal of claim 26, wherein the expression of the mutant of tau is inducible.

29. The transgenic non-human animal of claim 26, wherein a tet-off system is used for regulation of expression.

30. The transgenic non-human animal of claim 26, wherein the mutant of tau is K18ΔK280, K18ΔK280, I277P I308P, htau40ΔK280, or htau40ΔK280 I277P I308P.

31. (canceled)

32. A method to identify an agent that attenuates or inhibits aggregation of tau comprising testing the agent for its ability to attenuate or inhibit aggregation of tau within neurons in the transgenic non-human animal of claim 26.

33. The method of claim 32, wherein the agent is a compound selected from the group consisting of compounds 1 to 374 as shown in Table 1, a protein, an antibody, a fatty acid, a nucleotide, and a ribonucleic acid.

34. A method for identifying an agent for treating a neurodegenerative disease comprising testing the agent for its ability to attenuate or inhibit aggregation of tau in the transgenic non-human animal of claim 26.

35. A primary hippocampal cell culture from the transgenic non-human animal of claim 26.

36. A method for identifying an agent capable of inhibiting protein aggregate formation or capable of depolymerising protein aggregates, comprising contacting cells with the agent and determining a decrease in protein aggregate formation or a depolymerisation of protein aggregates, wherein the cells express a mutant of tau in an inducible fashion that polymerizes in the cell into aggregates which can be visualized by thioflavine S.

37. A method for identifying an agent capable of inhibiting protein aggregate formation or capable of depolymerising protein aggregates comprising contacting a transgenic non-human animals of claim 26 with the agent and determining a decrease in protein aggregate formation or a depolymerisation of protein aggregates.

38. The method of claim 36, wherein the agent is suitable for treating a neurodegenerative disease.

39. The method of claim 38, wherein the neurodegenerative disease is Alzheimer's disease, a taupathy, Parkinson's disease, fronto-temporal dementia, Pick's disease, corticobasal degeneration, or prion disease.

40. The method of claim 37, wherein the agent is suitable for treating a neurodegenerative disease.

41. The method of claim 40, wherein the neurodegenerative disease is Alzheimer's disease, a taupathy, Parkinson's disease, fronto-temporal dementia, Pick's disease, corticobasal degeneration, or prion disease.

42. A pharmaceutical composition comprising an inhibitor identified by the method of claim 21.

43. A method for inhibiting tau aggregation comprising the step of contacting tau with an effective amount of a composition of claim 42.

44. A method of for identifying an agent to attenuate or to inhibit aggregation of tau comprising the step of comparing aggregation of tau in a primary hippocampal cell culture from the transgenic non-human animal of claim 26 the presence or absence of the agent, wherein an increase in aggregation identifies the agent as an attenuator, and a decrease in aggregation identifies the agent as an inhibitor.
